(19) United States
(12) Patent Application Publication
(10) Pub. No.: US 2026/0028392 A1
(43) Pub. Date: Jan. 29, 2026

(54) HUMAN IGE MONOCLONAL ANTIBODIES TO PARASITIC WORM ANTIGENS AND USES THEREFOR

(71) Applicant: Vanderbilt University, Nashville, TN (US)

(72) Inventor: Scott A. SMITH, Nashville, TN (US)

(73) Assignee: Vanderbilt University, Nashville, TN (US)

(21) Appl. No.: 18/854,288

(22) PCT Filed: Mar. 28, 2023
(86) PCT No.: PCT/US2023/065026

§ 371 Date: Oct. 4, 2024

Related U.S. Application Data
(60) Provisional application No. 63/328,462, filed on Apr. 7, 2022.

(51) Int. Cl. C07K 16/18; G01N 33/68

(52) U.S. Cl. CPC C07K 16/18; G01N 33/6854; C07K 2317/565

(57) ABSTRACT

The present disclosure is directed to a method of detecting a helminth a antigen in a sample comprising (a) contacting a sample with human antibodies and fragments thereof, comprising clone-paired heavy and light chain CDR sequences, and (b) detecting a helminth antigen in said sample by binding of said human antibody or antibody fragment to a helminth antigen in said sample.

### PRIORITY CLAIM

This application claims benefit of priority to U.S. Provisional Application Ser. No. 63/328,462, filed Apr. 7, 2022, the entire contents of which are hereby incorporated by reference.

### FEDERAL FUNDING STATEMENT

This invention was made with government support under grant no. R01A130459 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Mar. 27, 2023, is named VBLTP0320WO.xml and is −296 kilobytes in size.

### BACKGROUND

### 1. Field of the Disclosure

The present disclosure relates generally to the fields of medicine, allergies and immunology. More particular, the disclosure relates to human IgE antibodies binding to parasitic worm antigens

### 2. Background

There is intense interest in the immunogenic triggers responsible for the rising epidemic of IgE-mediated allergic diseases; however, the ontogeny of the IgE antibody response in helminth infection has been poorly studied. While elevated serum levels of IgE antibodies are commonly found in tissue-invasive helminth infections, including the filarial nematodes that cause lymphatic filariasis, loiasis, and onchocerciasis (Jarrett & Bazin, 1974), there is limited understanding of antigenic triggers underlying this response and their protective function. Epidemiological data show that anti-parasite IgE responses are associated with a degree of immune-mediated protection in humans infected with hookworms (Bethony et al., 2005; Pritchard et al., 1995), Trichuris (Faulkner et al., 2002), Ascaris (Turner et al., 2005; McSharry et al., 1999) and schistosomes (Fitzsimmons et al., 2012; Pinot de Moira et al., 2010; Jiz et al., 2009; Dunne et al., 1992; 1997; Hagan et al., 1991; Rihet et al., 1991). However, early serologic studies characterize IgE antibodies as ‘nonspecific’ (non-antigen specific) (Jarrett et al., 1980), and others have focused on allergen-like proteins such as tropomyosin to explore overlapping IgE responses between helminth infection and allergic diseases (Sereda et al., 2008; Gazzinelli-Guimaraes et al., 2021; Santiago et al., 2011). The limitations of these studies stem from the difficulty of studying IgE using human sera, given the broad repertoire and exceedingly low concentration of antigen specific IgE. An important new way to dissect the function of IgE in the context of human helminth infection is to create naturally occurring IgE monoclonal antibodies (mAbs). The inventor has developed methods that allow for the generation of stable human hybridomas from the very rare population of helminth-specific IgE-encoding B cells in peripheral blood of infected human subjects. Here, the inventor performed an unbiased study of IgE antibodies associated with filarial infection and identified several filarial antigens targeted by the IgE response, which have strong diagnostic and vaccine potential. Moreover, the inventor suggest that IgE antibodies can act as an early detection system that can trigger a type one hypersensitivity response to constrain the parasite at a vulnerable early stage.

Filarial nematodes are threadlike worms with complex life cycles in which the adult worms reside in lymphatic or subcutaneous tissue of their host. These parasites are responsible for causing lymphatic filariasis (LF; Wuchereria bancrofti, Brugia malayi, Brugi timori), onchocericasis (O. volvulus) and/or loiasis (Loa loa). These chronic infections are responsible for an extraordinary degree of morbidity and collectively affect more that 180 million people worldwide. The canonical host immune response to these particular tissue-invasive helminths is of the T-helper 2 (Th2) type and involves the production of cytokines, interleukin (IL)-4, IL-5, IL-9, IL-10, and IL-13; the antibody isotypes immunoglobulin G1 (IgG1), IgG4, and IgE; and expanded populations of eosinophils, basophils, mast cells, type 2 innate lymphoid cells, and alternatively activated macrophages (Nutman, 2015). Interestingly, filarial infection in travelers and tropical pulmonary eosinophilia (TPE; an unusual syndrome seen in W. bancrofti and B. malayi infection) are associated with both extraordinarily elevated IgE levels and “allergic” symptomatology (Ottesen & Nutman, 1992). Thus, having insights into the antigens driving IgE and mediating pathology is of paramount interest.

### SUMMARY

Thus, in accordance with the present disclosure, there is provided a method of detecting a helminth antigen in a sample comprising (a) contacting a sample with an antibody or antibody fragment comprising clone-paired heavy and light chain CDR sequences from Tables 3 and 4, respectively; and (b) detecting a helminth antigen in said sample by binding of said antibody or antibody fragment to a helminth antigen in said sample. The sample may be a biologic fluid, such has a biologic culture, media or culture supernatant, blood, sputum, tears, saliva, mucous or serum, semen, cervical or vaginal secretions, amniotic fluid, placental tissues, urine, exudate, transudate, tissue scrapings or feces, a food or tissue sample, a drug formulation, a biologic therapeutic formulation or a vaccine stock. Detection may comprise ELISA, RIA, lateral flow assay or Western blot. The method may further comprise performing steps (a) and (b) a second time and determining a change in helminth antigen levels as compared to the first assay. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment.

In another embodiment, there is provided a method of detecting IgE anti-helminth antigen antibodies in a sample comprising (a) contacting a sample suspected of containing an IgE anti-helminth antigen antibody with a helminth antigen in a competitive assay with an antibody or antibody fragment comprising clone-paired heavy and light chain CDR sequences from Tables 3 and 4, respectively; and (b) detecting competition for binding of helminth antigen by a suspected IgE anti-helminth antigen in said sample by an antibody or antibody fragment comprising clone-paired heavy and light chain CDR sequences from Tables 3 and 4, respectively. The helminth antigen may be selected from SXP-1, MIF, 24kDaSPro, Gp15/400, TTR_4, TTR_32, TTR_61, TTR_76, TTR_62, TTR_79, TTR_07, TTR_08, TTR_16, TTR_90, TTR_77, TTR_47, TTR_41, TTR_95, TTR_05, or a homolog thereof.

In yet another embodiment, there is provided a method of preventing or treating parasitic worm infection in a subject comprising delivering to said subject an antibody or antibody fragment comprising clone-paired heavy and light chain CDR sequences from Tables 3 and 4, respectively. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody or antibody fragment may IgE or IgG. For IgG antibodies, they may comprise an Fc portion mutated to alter (eliminate or enhance) FcR interactions, to increase half-life and/or increase therapeutic efficacy or is glycan modified to alter (eliminate or enhance) FcR interactions, such as an Fc portion that contains a LALA, LALA-PG, N297, GASD/ALIE, DHS, YTE or LS mutation. The antibody may be a chimeric antibody or a bispecific antibody. Delivering may comprise antibody or antibody fragment administration, or genetic delivery with an RNA or DNA sequence or vector encoding the antibody or antibody fragment.

In still yet another embodiment, there is provided a monoclonal antibody, wherein the antibody or antibody fragment comprises clone-paired heavy and light chain CDR sequences from Tables 3 and 4, respectively. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody or antibody fragment may be IgE or IgG. The IgG antibody may comprise an Fc portion mutated to alter (eliminate or enhance) FcR interactions, to increase half-life and/or increase therapeutic efficacy or is glycan modified to alter (eliminate or enhance) FcR interactions, such as an Fc portion that contains a LALA, LALA-PG, N297, GASD/ALIE, DHS, YTE or LS mutation. The antibody may be a chimeric antibody or a bispecific antibody. The antibody or antibody fragment may further comprise a cell penetrating peptide and/or is an intrabody.

In a further embodiment, there is provided a hybridoma or engineered cell encoding an antibody or antibody fragment wherein the antibody or antibody fragment comprises clone-paired heavy and light chain CDR sequences from Tables 3 and 4, respectively. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody or antibody fragment may be an IgE or IgG. The IgG antibody may comprise an Fc portion mutated to alter (eliminate or enhance) FcR interactions, to increase half-life and/or increase therapeutic efficacy or is glycan modified to alter (eliminate or enhance) FcR interactions, such as an Fc portion that contains a LALA, LALA-PG, N297, GASD/ALIE, DHS, YTE or LS mutation. The antibody may be a chimeric antibody or a bispecific antibody.

In yet a further embodiment, there is provided a vaccine formulation comprising one or more IgE antibodies or antibody fragments comprising clone-paired heavy and light chain CDR sequences from Tables 3 and 4, respectively. At least one antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. At least one antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. At least one antibody fragment may be a recombinant scFv (single chain fragment variable) antibody, Fab fragment, F(ab′)₂ fragment, or Fv fragment. The antibody or antibody fragment may by an IgE or IgG. The IgG antibody may comprise an Fe portion mutated to alter (eliminate or enhance) FcR interactions, to increase half-life and/or increase therapeutic efficacy or is glycan modified to alter (eliminate or enhance) FcR interactions, such as an Fc portion that contains a LALA, LALA-PG, N297, GASD/ALIE, DHS, YTE or LS mutation. The antibody may be a chimeric antibody or a bispecific antibody.

In still yet a further embodiment, there is provided a vaccine formulation comprising one or more expression vectors encoding a first antibody or antibody fragment, wherein the antibody or antibody fragment comprises clone-paired heavy and light chain CDR sequences from Tables 3 and 4, respectively. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody or antibody fragment may be an IgE or IgG. The IgG antibody may comprise an Fc portion mutated to alter (eliminate or enhance) FcR interactions, to increase half-life and/or increase therapeutic efficacy or is glycan modified to alter (eliminate or enhance) FcR interactions, such as an Fe portion that contains a LALA, LALA-PG, N297, GASD/ALIE, DHS, YTE or LS mutation. The antibody may be a chimeric antibody or a bispecific antibody. The expression vector(s) may be Sindbis virus or VEE vector(s). The vaccine formulation may be formulated for delivery by needle injection, jet injection, or electroporation. The vaccine formulation may further comprise one or more expression vectors encoding for a second antibody or antibody fragment, such as a distinct antibody or antibody fragment as defined herein.

Also provided are:
a human monoclonal antibody or antibody fragment, or hybridoma or engineered cell producing the same, wherein said antibody binds to a parasitic worm antigen selected from SXP-1, MIF, 24kDaSPro, Gp15/400, TTR_4, TTR_32, TTR_61, TTR_76, TTR_62, TTR_79, TTR_07, TTR_08, TTR_16, TTR_90, TTR_77, TTR_47, TTR_41, TTR_95, TTR_05, or a homolog thereof;
a method of detecting a helminth antigen in a sample comprising (a) contacting a sample suspected of containing a helminth antigen with an antibody or antibody fragment comprising clone-paired heavy and light chain CDR sequences from Tables 3 and 4, respectively; and (b) detecting binding of said antibody or antibody fragment to an antigen in said sample;
a vaccine composition comprising one or more parasitic worm antigens selected from SXP-1, MIF, 24kDaSPro, Gp15/400, TTR_4, TTR_32, TTR_61, TTR_76, TTR_62, TTR_79, TTR_07, TTR_08, TTR_16, TTR_90, TTR_77, TTR_47, TTR_41, TTR_95, TTR_05, or a homolog thereof; and a method of immunizing a subject against a parasitic worm infection comprising administering to said subject a vaccine composition according to the preceding paragraph.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C: Characterization of IgE mAbs isolated from peripheral blood of subjects with filarial infection. (FIG. 1A) Heatmap representing IgE binding to B. malayi ImmunoCAP (considered positive if >1 kUA/L) and B. malayi and D. immitis binding in ELISA (- no binding detected; +binding 3-10 times background; ++binding -100 times background; +++binding >100 times background) and immunoblot (WB) (- no binding; +++presence of a clear band). MAbs are ordered by ImmunoCAP binding value. Data are representative of at least two independent experiments. (FIG. 1B) Immunoblot analysis of IgE mAb binding to protein in somatic extracts from B. malayi. (FIG. 1C) Analysis as in FIG. 1B using D. immitis extracts. For FIG. 1B and FIG. 1C only immunoblot-positive antibodies are shown.

FIGS. 2A-D: Identification of filarial antigens targeted by IgE mAbs. (FIG. 2A) Analysis of specificity of each IgE mAb for its target antigen by IP followed by immunoblot. A filarial worm somatic extract was immunoprecipitated with IgE mAbs. Whole somatic extract (Pre-IP), IP product and extract after IP (Post-IP) were then blotted with the same IgE mAb. Only immunoblot-positive antibodies are shown. (FIG. 2B), Shown is the number of peptide spectrum matches (PSMs) identified for each antigen by mass spectrometry analysis. (FIG. 2C) Specificity validation of each IgE mAb for its target by ELISA. Data obtained in triplicate are shown as the mean±SEM and are representative of three experiments. Calculated EC50 values are shown on the graph. (FIG. 2D) Specificity validation of each IgE mAb for its target by immunoblot analysis.

FIGS. 3A-C: TTR family proteins are the prominent filarial antigens targeted by IgE antibodies and have diagnostic potential. (FIG. 3A) ELISA analysis depicting TTR-specific antibody cross-reactivity profile to multiple TTR proteins. MAb 12D4, a human IgE mAb specific for WbSXP-1, served as negative control. OD, optical density. (FIG. 3B) Proportion of filaria-specific antibodies binding different antigens. (FIG. 3C) Binary heatmap showing presence (red) or absence (black) of antibody in indicated patient sera against filarial antigens tested in immunoblot analysis. Two serum samples (284 and 245) from respective mold- and shellfish-allergic patients served as negative controls.

FIGS. 4A-D: IgH and IgL gene sequence analysis of filaria-specific IgE antibodies. Sixteen filaria-specific IgE antibodies were found to have unique sequences and were analyzed for genetic features. (FIG. 4A) Heatmap showing the total number of unique sequences with corresponding V and J genes. (FIG. 4B) CDR3 amino acid length distribution for heavy and light chains with indicated mean. (FIG. 4C) IgH and IgL CDR3 amino acid number was determined using the ImMunoGeneTics (IMGT) database. (FIG. 4D) Number of SHMs in the VH and VL chain of filaria-specific mAb genes.

FIGS. 5A-B: Filarial antigens induce anaphylaxis in IgE mAb-sensitized human FcERIa-transgenic mice. Mice were sensitized with a 100 gg i.p. injection of human IgE mAb. Three days later, mice were injected i.p. with 50 g of filarial antigens or a control allergen (10% peanut extract). Body temperature over 90 minutes was monitored using an implanted temperature probe. (FIG. 5A) Mice were sensitized with a 100 g i.p. injection of human IgE mAb 5H1 and challenged with TTR_76, TTR_79, or 10% peanut extract. (FIG. 5B) Mice were sensitized with 5H1 or 18H7 or both and challenged with TTR_79, TTR_62, or 10% peanut extract. Change in body temperature of experimental compared to control allergen groups at each time point using a paired 2-tailed t test assuming unequal variance. Time points with calculated P values <0.05 are highlighted by an asterisk. Data are means±SEM of each experimental group. The number of mice (n) for each experimental group is shown.

FIGS. 6A-B: Schematic showing human IgE antibody generation and workflow of filarial antigen discovery. (FIG. 6A) PBMCs were isolated from blood and processed for in vitro expansion of B cells prior to screening for IgE production by ELISA. IgE-producing B cells were fused with a myeloma fusion partner followed by HAT selection and single cell sorting. Each hybridoma was expanded in serum-free media for large-scale mAb production and affinity purification. (FIG. 6B) IgE mAbs were tested for reactivity to B. malayi and D. immitis using ELISA, immunoblot, and B. malayi ImmunoCAP. IgE mAbs showing reactivity in multiple assays were subjected to IP and mass spectrometry analysis for antigen discovery. Filarial antigens were expressed in E. coli as their homolog W bancrofti antigens. IgE mAbs were validated against recombinant antigens by ELISA and immunoblot analysis.

FIG. 7: Cross-reactivity analysis of IgE mAbs from patients with LF against common allergens. Heatmap of IgE binding to an ISAC allergen array. MAbs were used at an estimated concentration of 1-10 g/mL against 112 common allergens. CTR02 is a positive control included in the immunoCAP ISAC assay.

FIG. 8: Immunoblot analysis of a D. immitis protein somatic extract and B. malayi culture using mAb 2E6. Lane 1 shows the molecular weight marker. Lane 2 is run with D. immitis somatic whole worm extract. Lane 3 contains concentrated culture media in which B. malayi adult male and female worms were kept for 7 days, containing only excreted proteins.

FIGS. 9A-C: Extent of 12D4 and 4E1 cross-reactivity against SXP-1 proteins from W. bancrofti, B. malayi and D. immitis. (FIG. 9A) Immunoblot analysis of mAb 12D4 against recombinant WbSXP-1 and BmSXP-1 as well as a D. immitis protein somatic extract containing DiSXP-1. (FIG. 9B) Immunoblot analysis of mAb 4E1 against recombinant WbSXP-1 and DiSXP-1. (FIG. 9C) Amino acid sequence alignment of WbSXP-1 with homologous proteins in B. malayi (AAA27864.1) and D. immitis (JR903413.1). Amino acid conservation across aligned proteins is indicated as follows: “*” identical; “:” strongly similar; “.” weakly similar. [SEQ ID NOS: 337-339]FIGS. 10A-B: Phylogenetic tree of W. bancrofti TTR proteins. (FIG. 10A) All sequences reported for WbTTR proteins were collected from NCBI, and a maximum-likelihood phylogenetic tree of TTR proteins was assembled using the MUSCLE algorithm, with omission of partial and repetitive sequences reported in NCBI. Proteins chosen for recombinant expression are boxed in red. (FIG. 10B) Pairwise percent identity of W. bancrofti TTR proteins.

FIG. 11: Cross-reactivity of TTR-specific IgE mAbs against different TTR proteins. Fifteen WbTTR proteins were expressed in Shuffle T7 E. coli and used for immunoblot analysis. Protein expression was verified by immunoblot using an anti-His polyclonal antibody prior to cross-reactivity analysis.

FIG. 12: Amino acid sequence alignment of WbTTR proteins bound by 5H1 or 18H7 IgE mAbs. Amino acid sequence alignment of all WbTTR proteins bound by at least one human IgE mAb. Amino acid conservation across aligned proteins is indicated as follows: “*” identical; “:” strongly similar; “.” weakly similar. [SEQ ID NOS: 340-353]FIGS. 13A-G: Immunoblot analysis of sera from patients infected with filaria against different filarial antigens. Filarial proteins were expressed in Shuffle T7 E. coli and used for immunoblot analysis against sera from patients with filarial infection. Sera (284, 245) from allergic subjects served as negative controls. FIG. 13A, P1; FIG. 13B, P3; FIG. 13c, P5; FIG. 13D, P8; and FIG. 13E, P9. FIG. 13F, 284 allergic control subject: cat, dog, and dust mite; FIG. 13G, 245 allergic control subject: peanut, cashew, and dust mite.

### DESCRIPTION OF ILLUSTRATIVE EMBODIMENTS

As discussed above, much of the understanding of the immunogenic triggers and effects of the IgE antibody response comes from studies of allergy, though little is known about the natural immunogenic targets seen following parasitic worm infections. Here, the inventor directly addresses the question of IgE antibody specificity in the context of filarial infections, vector-borne helminth infections affecting more than 800 million people worldwide that typically evoke a marked IgE response. This unbiased analysis of IgE antibodies obtained from filaria-infected human subjects revealed all immunodominant worm antigens to be excreted/secretory (E/S) proteins, with the strongest IgE-inducers identified as the transthyretin-related proteins - a previously uncharacterized family of related antigens. Functional analysis in mice revealed that filaria-specific IgE passively induces anaphylactic reactions and suggests that helminth-specific IgE antibodies antagonize infection by inducing a type one hypersensitivity response. Antigens identified here have diagnostic and vaccine potential against often-overlooked infections with few treatment options. Moreover, this work reveals the targeting strategy of the type 2 immune response/IgE antibody response and suggests the origin of modern pathological diseases caused by IgE.

These and other aspects of the disclosure are described in detail below.

### I. IgE Antibodies and Recognized Pathogens

### A. Biology

Immunoglobulin E (IgE), first discovered in 1966, is a kind of antibody (or immunoglobulin (Ig) “isotype”) that has only been found in mammals. IgE is synthesised by plasma cells. Monomers of IgE consist of two heavy chains (E chain) and two light chains, with the ε chain containing 4 Ig-like constant domains (Cε1-Cε4). IgE's main function is immunity to parasites such as helminths like Schistosoma mansoni, Trichinella spiralis, and Fasciola hepatica. IgE is utilized during immune defense against certain protozoan parasites such as Plasmodium falciparum:.

IgE also has an essential role in type I hypersensitivity, which manifests in various allergic diseases, such as allergic asthma, most types of sinusitis, allergic rhinitis, food allergies, and specific types of chronic urticaria and atopic dermatitis. IgE also plays a pivotal role in responses to allergens, such as: anaphylactic drugs, bee stings, and antigen preparations used in desensitization immunotherapy.

Although IgE is typically the least abundant isotype-blood serum IgE levels in a normal (“non-atopic”) individual are only 0.05% of the Ig concentration, compared to 75% for the IgGs at 10 mg/ml, which are the isotypes responsible for most of the classical adaptive immune response-it is capable of triggering the most powerful inflammatory reactions.

IgE primes the IgE-mediated allergic response by binding to Fc receptors found on the surface of mast cells and basophils. Fc receptors are also found on eosinophils, monocytes, macrophages and platelets in humans. There are two types of FcE receptors, FcR1 (type I Fcs receptor), the high-affinity IgE receptor, and FcεRII (type II Fce receptor), also known as CD23, the low-affinity IgE receptor. IgE can upregulate the expression of both types of Fc receptors. FcR1 is expressed on mast cells, basophils, and the antigen-presenting dendritic cells in both mice and humans. Binding of antigens to IgE already bound by the FcεRI on mast cells causes cross-linking of the bound IgE and the aggregation of the underlying FcεRI, leading to the degranulation and the release of mediators from the cells. Basophils, upon the cross-linking of their surface IgE by antigens, release type 2 cytokines like interleukin-4 (IL-4) and interleukin-13 (IL-13) and other inflammatory mediators. The low-affinity receptor (FcεRII) is always expressed on B cells; but IL-4 can induce its expression on the surfaces of macrophages, eosinophils, platelets, and some T cells.

There is much speculation into what physiological benefits IgE contributes, and, so far, circumstantial evidence in animal models and statistical population trends have hinted that IgE may be beneficial in fighting gut parasites such as Schistosoma mansoni, but this has not been conclusively proven in humans. Epidemiological research shows that IgE level is increased when infected by Schistosoma mansoni, Necator americanus, and nematodes in human. It is most likely beneficial in removal of hookworms from the lung.

Regulation of IgE levels through control of B cell differentiation to antibody-secreting plasma cells is thought to involve the “low-affinity” receptor FcεRII, or CD23. CD23 may also allow facilitated antigen presentation, an IgE-dependent mechanism whereby B cells expressing CD23 are able to present allergen to (and stimulate) specific T helper cells, causing the perpetuation of a Th2 response, one of the hallmarks of which is the production of more antibodies.

### B. Parasitic Worms

Parasitic worms, also known as helminths, are large macroparasites; adults can generally be seen with the naked eye. Many are intestinal worms that are soil-transmitted and infect the gastrointestinal tract. Other parasitic worms such as schistosomes reside in blood vessels. Some parasitic worms, including leeches and monogeneans, are ectoparasites - thus, they are not classified as helminths, which are endoparasites.

Parasitic worms live in and feed in living hosts. They receive nourishment and protection while disrupting their hosts' ability to absorb nutrients. This can cause weakness and disease in the host. Parasitic worms cannot reproduce entirely within their host's body; they have a life cycle that includes some stages that need to take place outside of the host. Helminths are able to survive in their mammalian hosts for many years due to their ability to manipulate the host's immune response by secreting immunomodulatory products. All parasitic worms produce eggs during reproduction. These eggs have a strong shell that protects them against a range of environmental conditions. The eggs can therefore survive in the environment for many months or years.

Many of the worms referred to as helminths are intestinal parasites. An infection by a helminth is known as helminthiasis, helminth infection, or intestinal worm infection. Helminths are a group of organisms which share a similar form but are not necessarily related as part of evolution. The term “helminth” is an artificial term. There is no real consensus on the taxonomy (or groupings) of the helminths, particularly within the nematodes. The term “helminth” contains a number of phyla, many of which are completely unrelated. However, for practical considerations the term is currently used to describe four phyla with superficial similarities:
Annelida (ringed or segmented worms)
Platyhelminthes (flatworms)
Nematoda (roundworms)
Acanthocephala (thorny-headed worms).

The phylum Platyhelminthes includes two classes of worms of particular medical significance: the cestodes (tapeworms) and the trematodes (flukes and blood flukes), depending on whether or not they have segmented bodies. There may be as many as 300,000 species of parasites affecting vertebrates, and as many as 300 affecting humans alone. Helminths of importance in the sanitation field are the human parasites and are classified as Nemathelminthes (nematodes) and Platyhelminthes, depending on whether they possess a round or flattened body, respectively.

The lifetime of adult worms varies tremendously from one species to another but is generally in the range of 1 to 8 years (see following table). This lifetime of several years is a result of their ability to manipulate the immune response of their hosts by secreting immunomodulatory products.

Helminths can be either hermaphroditic (having the sex organs of both sexes), like tapeworms and flukes (not including the blood fluke), or have their sexes differentiated, like the roundworms. All helminths produce eggs (also called ova) for reproduction. Generally, thousands or even hundreds of thousands of eggs are produced each time the female worm deposits its eggs - a process called oviposition. There is a large variation in the number of eggs produced by different species of worm at one time; it varies in the range of 3,000 to 700,000. The frequency of egg deposition from an adult helminth is generally daily and can occur up to six times per day for some Taenia species. Adult trematodes lay smaller numbers of eggs compared to cestodes or nematodes. However, the egg develops into a miracidia from which thousands of cercariae, or swimming larvae, develop. This means that one egg may produce thousands of adult worms. Helminth eggs remain viable for 1-2 months in crops and for many months in soil, fresh water, and sewage, or even for several years in feces, fecal sludge (historically called night soil), and sewage sludge - a period that is much longer compared to other microorganisms. Eggs can reach the soil when polluted wastewater, sewage sludge or human waste are used as fertilizer. Such soil is often characterized by moist and warm conditions. Therefore, the risk of using contaminated wastewater and sludge in agricultural fields is a real problem, especially in poor countries, where this practice is prevalent. Helminth eggs are regarded as the main biological health risk when applying sewage sludge, fecal sludge or fecal matter on agricultural soils. The eggs are the infective stage of the helminths' life cycle for causing the disease helminthiasis.

Helminth eggs are resistant to various environmental conditions due to the composition of the egg shell. Each helminth egg species has 3 to 4 layers with different physical and chemical characteristics:
the 1 to 2 outer layers formed of mucopolysaccharides and proteins,
the middle layers consisting of chitinous material and serve to give structure and mechanical resistance to the eggs, and
the inner layer composed of lipids and proteins useful to protect eggs from desiccation, strong acid and bases, oxidants and reductive agents as well as detergent and proteolytic compounds

Due to this strong shell, helminth eggs or ova remain viable in soil, fresh water and sewage for many months. In feces, fecal sludge and sewage sludge they can even remain viable for several years. Helminth eggs of concern in wastewater used for irrigation have a size between 20 and 90 μm and a relative density of 1.06-1.23. It is very difficult to inactivate helminth eggs, unless temperature is increased above 40° C. or moisture is reduced to less than 5%. Eggs that are no longer viable do not produce any larvae. In the case of Ascaris lumbricoides (giant roundworm), which has been considered the most resistant and common helminth type, fertilized eggs deposited in soil are resistant to desiccation but are, at this stage of development, very sensitive to environmental temperatures: The reproduction of a fertilized egg within the eggshell develops at an environmental soil temperature about 25° C. which is lower than the body temperature of the host (i.e., 37° C. for humans). However, development of the larvae in the egg stops at temperatures below 15.5° C., and eggs cannot survive temperatures much above 38° C. If the temperature is around 25° C., the infectiousness occurs after nearly 10 days of incubation.

Larvae hatch from eggs, either inside or outside the host, depending on the type of helminth. For eggs in moist soil at optimal temperature and oxygen levels, the embryo develops into an infective larva after 2 to 4 weeks, named “second-stage larva”. Once ingested by a host, this larva has the ability to get out of the egg, hatch in the small intestine and migrate to different organs. These infective larvae (or “infective eggs”) may remain viable in soil for two years or longer. The process of larval maturation in the host can take from about two weeks up to four months, depending on the helminth species.

For the purpose of setting treatment standards and reuse legislation, it is important to be able to determine the amount of helminth eggs in an environmental sample with some accuracy. The detection of viable helminth eggs in samples of wastewater, sludge or fresh feces (as a diagnostic tool for the infection helminthiasis) is not straight forward. In fact, many laboratories in developing countries lack the right equipment or skilled staff required to do so. An important step in the analytical methods is usually the concentration of the eggs in the sample, especially in the case of wastewater samples. A concentration step may not be required in samples of dried feces, e.g. samples collected from urine-diverting dry toilets.

For medical purposes, the exact number of helminth eggs is less important and therefore most diagnoses are made simply by identifying the appearance of the worm or eggs in feces. Due to the large quantity of eggs laid, physicians can diagnose using as few as one or two fecal smears. The Kato technique (also called the Kato-Katz technique) is a laboratory method for preparing human stool samples prior to searching for parasite eggs. Eggs per gram is a laboratory test that determines the number of eggs per gram of feces in patients suspected of having a parasitological infection, such as schistosomiasis.

### II. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{HI}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. The gamma and alpha classes are further divided into subclasses on the basis of relatively minor differences in C_{H} sequence and function, humans express the following subclasses: IgG1, IgG2, IgG3, IgG4, IgAQ1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-(Hi), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

### A. General Methods

It will be understood that monoclonal antibodies binding to helminth antigens will have several applications. These include the production of diagnostic kits for use in detecting and diagnosing helminth antigens contamination and helminth antigen antibody levels in subjects, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as ASO1 or AS03). Additional experimental forms of inoculation to induce helminth antigens-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes forbetter efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims.

The term “epitope” refers to a site on an antigen to which B and/or T cells respond. The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-helminth antigen antibody, the above-described binding methodology is performed in two orientations: In a first orientation, the reference antibody is allowed to bind to helminth antigens under saturating conditions followed by assessment of binding of the test antibody to the complex. In a second orientation, the test antibody is allowed to bind to the helminth antigens molecule under saturating conditions followed by assessment of binding of the reference antibody to the complex. If, in both orientations, only the first (saturating) antibody is capable of binding to the helminth antigens, then it is concluded that the test antibody and the reference antibody compete for binding to helminth antigens. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. Such antibodies may be produced by the clones discussed below in the Examples section using methods described herein.

In another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 1 and the amino acid sequences of Table 2.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. 0. (1978) A model of evolutionary change in proteins—Matrices for detecting distant relationships. In Dayhoff, M. 0. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity. Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002), J. Biol. Chem. 277(30): 26733-26740; Davies J. et al. (2001), Biotechnology & Bioengineering 74(4): 288-294). Methods of altering carbohydrate contents are known to those skilled in the art, see, e.g., Wallick, S. C. et al. (1988), J. Exp. Med. 168(3): 1099-1109; Tao, M. H. et al. (1989), J. Immunol. 143(8): 2595-2601; Routledge, E. G. et al. (1995), Transplantation 60(8):847-53; Elliott, S. et al. (2003), Nature Biotechnol. 21:414-21; Shields, R. L. et al. (2002), J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (Nlmψ) outperforms several other nucleoside modifications and their combinations in terms of translation capacity. In addition to turning off the immune/eIF2α phosphorylation-dependent inhibition of translation, incorporated N1mT nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)2) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (-3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG1 can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fe mutations can also be introduced and engineered to alter their interaction with the neonatal Fe receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

Class Switch. The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency. Modifications in the Fc region can be introduced to extend the in vivo half-life of the antibody, or to alter Fc mediated functions such as complement activation, antibody dependent cellular cytotoxicity (ADCC), and FcR-mediated phagocytosis.

Of central importance to the present disclosure is isotype modification involving changing a naturally occurring human IgE isotype variable sequence to an IgG isotype. By making this unnatural modification of the IgE antibody, a pathogenic molecule can be made to possess therapeutic functions. Aside from the theoretical benefit that IgE isotype antibodies may have in control of helminth infections, IgE antibodies are necessary for causing IgE-mediated allergy. The function of an IgE antibody is conveyed through its Fe region, which directs binding of the antibody to specific Fc receptors on various cells. By changing a natural human IgE to an IgG, one completely alters the Fe receptors that can be engaged - this has never been shown to occur naturally in humans since the IgG isotypes are deleted from the B cell DNA when it class-switched to IgE. It is the IgE antibody's ability to bind the Fc receptors, FcεRI and FcRII, which endow its pathogenic function. By engineering a human IgE variable sequence into an IgG antibody isotype, the pathogenic molecule can no longer perform its harmful functions. Additionally, the engineered IgG antibody can then provide new, therapeutic functions through engagement with various Fey receptors, such as those found on the mast cell, including FcγyRIIB. IgG antibodies that bind FcγRIIB on the surface of mast cells result in inhibitory signaling and inhibition of mediator release. For example, an allergen specific IgE antibody bound to FcεRI on mast cells will signal the release of inflammatory mediators upon binding its specific allergen - resulting in the diseases associated with allergy. However, an IgG made from the allergen-specific IgE variable sequences would bind FcγRIIB on mast cells and inhibit mediator release upon binding the specific disease inciting allergen.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fe glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fe gamma RIII compared to the same antibody without the substantially homogeneous GNGN glycoform and with GO, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. 0-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2)N-linked glycosylation,
3) Asn deamidation,
4) Asp isomerization,
5) SYE truncation,
6) Met oxidation,
7) Trp oxidation,
8)N-terminal glutamate,
9) Integrin binding,
10) CD11c/CD18 binding, or
11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21(2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fe portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pls). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Ga14 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-a, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (CH1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986). According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998). doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fe region or a hinge region. In this scenario, the antibody will comprise an Fe region and three or more antigen binding sites amino-terminal to the Fe region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: V_{H}-CH1-flexible linker-V_{H}-CH1-Fc region chain; or V_{H}-CH1-V_{H}-CH1-Fe region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a CL domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a V_{H}/V_{L} exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains V_{L} and V_{H} of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CHI of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CHI of the second Fab molecule under b) the amino acid at position 147 or the amino acid at position 213 is substituted by a negatively charged amino acid (numbering according to Kabat EU index).

The antibody may not comprise both modifications mentioned under i) and ii). The constant domains CL and CHI of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CHI of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CHI of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

### F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain - linker - heavy chain, the native signal of the light-chain is used The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the C_{H2}C_{H3} region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 - chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

### G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex - amino acid, linker and cytotoxic agent - now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

### H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

### I. Intrabodies

In a particular embodiment, the antibody is a recombinant antibody that is suitable for action inside of a cell - such antibodies are known as “intrabodies.” These antibodies may interfere with target function by a variety of mechanisms, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

### J. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fe portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Induction of Immune Response

### A. Formulation and Administration

The present disclosure provides pharmaceutical compositions comprising anti-helminth antigens antibodies and helminth antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### 2. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fe region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

### 3. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

### IV. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵′chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Patent 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fe region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fe region have also been disclosed in the literature (O'Shannessy et al., 1987). This approach has been reported to produce diagnostically and therapeutically promising antibodies which are currently in clinical evaluation.

### V. Immunodetection Methods

In still further embodiments, the present disclosure concerns immunodetection methods for binding, purifying, removing, quantifying and otherwise generally detecting helminth antigens containing molecules. While such methods can be applied in a traditional sense, another use will be in quality control and monitoring of vaccine and other antigen stocks, where antibodies according to the present disclosure can be used to assess the amount of helminth antigens in vaccine stocks. Alternatively, the methods may be used to screen various antibodies for appropriate/desired reactivity profiles.

Other immunodetection methods include specific assays for determining the presence of anti-helminth antigens antibodies in a subject. A wide variety of assay formats are contemplated, but specifically those that would be used to detect anti-helminth antigens antibodies in a fluid obtained from a subject, such as saliva, blood, plasma, sputum, semen or urine. The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of anti-helminth antigens antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing anti-helminth antigens antibodies and contacting the sample with a first antibody in accordance with the present disclosure, as the case may be, under conditions effective to allow the formation of immunocomplexes.

These methods include methods for purifying anti-helminth antigens antibodies from a sample. The antibody will preferably be linked to a solid support, such as in the form of a column matrix, and the sample suspected of containing the anti-helminth antigens antibodies will be applied to the immobilized antibody. The unwanted components will be washed from the column, leaving the anti-helminth antigens antibodies immunocomplexed to the immobilized antibody, which is then collected by removing the organism or antigen from the column.

The immunobinding methods also include methods for detecting and quantifying the amount of anti-helminth antigens antibodies in a sample and the detection and quantification of any immune complexes formed during the binding process. Here, one would obtain a sample suspected of containing anti-helminth antigens antibodies and contact the sample with an antibody that competes with anti-helminth antigens antibodies, followed by detecting and quantifying the amount of immune complexes formed under the specific conditions. In terms of antigen detection, the biological sample analyzed may be any sample that is suspected of containing anti-helminth antigens antibodies, such as a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to helminth antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, for example, with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the anti-helminth antigens antibodies is added to the wells. After binding and washing to remove non-specifically bound immune complexes, the bound antigen may be detected. Detection may be achieved by the addition of another anti-helminth antigen antibody that is linked to a detectable label. This type of ELISA is a simple “sandwich ELISA.” Detection may also be achieved by the addition of a second anti-helminth antigens antibodies, followed by the addition of a third antibody that has binding affinity for the second antibody, with the third antibody being linked to a detectable label.

In another exemplary ELISA, the samples suspected of containing the helminth antigens are immobilized onto the well surface and then contacted with the anti-helminth antigens antibodies of the disclosure. After binding and washing to remove non-specifically bound immune complexes, the bound anti-helminth antigens antibodies are detected. Where the initial anti-helminth antigens antibodies are linked to a detectable label, the immune complexes may be detected directly. Again, the immune complexes may be detected using a second antibody that has binding affinity for the first anti-helminth antigen antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween). After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of anti-helminth antigens antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventor proposes the use of labeled anti-helminth antigens antibodies to determine the amount of anti-helminth antigens antibodies in a sample. The basic format would include contacting a known amount of anti-helminth antigen antibody (linked to a detectable label) with sample containing anti-helminth antigens antibodies, such as attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein. Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

### D. Kits

In still further embodiments, the present disclosure concerns kits for use with the methods described above. As the antibodies may be used to detect helminth antigens or anti-helminth antigens antibodies may be included in the kit. The kits will thus comprise, in suitable container means, a first antibody that binds to helminth antigens, and optionally an immunodetection reagent.

In certain embodiments, the anti-helminth antigens antibodies may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the helminth antigens or anti-helminth antigens antibodies, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

Antibodies and fragments thereof as described in the present disclosure may also be used in a kit for monitoring allergic reaction by detecting the presence of anti-helminth antigens antibodies.

### E. Vaccine and Antigen Quality Control Assays

The present disclosure also contemplates the use of antibodies and antibody fragments as described herein for use in assessing the presence of an helminth antigens antigen in a sample. Medicinal products like vaccines, biologics and chemical drugs can contain contaminants including helminth antigens and have the capacity to vary widely from preparation to preparation. They are also administered to healthy as well as diseased individuals, including children, and thus a strong emphasis must be placed on their quality to ensure, to the greatest extent possible, that they are efficacious in preventing or treating life-threatening disease, without themselves causing harm.

The increasing globalization in the production and distribution of medicines has opened new possibilities to better manage public health concerns but has also raised questions about the equivalence and interchangeability of medicine procured across a variety of sources. International standardization of starting materials, of production and quality control testing, and the setting of high expectations for regulatory oversight on the way these products are manufactured and used, have thus been the cornerstone for continued success. It remains a field in constant change, and continuous technical advances in the field offer a promise of developing potent new weapons against the oldest public health threats, as well as new ones, but also put a great pressure on manufacturers, regulatory authorities, and the wider medical community to ensure that products continue to meet the highest standards of quality attainable.

Thus, one may obtain a product from any source or at any point during a manufacturing process. The quality control processes may therefore begin with preparing a sample for an immunoassay that identifies binding of an antibody or fragment disclosed herein to an helminth antigens antigen. Such immunoassays are disclosed elsewhere in this document, and standards for finding the sample to contain helminth antigens antigen may be established by regulatory agencies.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Data reporting. No statistical methods were used to predetermine sample size. Experiments were not randomized, and investigators were not blinded to allocation during experiments and outcome assessment.

Research subjects. The inventor analyzed seven subjects with a clinical history of lymphatic filariasis, loiasis, or onchocerciasis, two diagnosed with tropical pulmonary eosinophilia (TPE). Relevant clinical information is summarized in Supplementary Table 1. The study was approved by the Institutional Review Board of Vanderbilt University Medical Center (IRB 170308). Samples were obtained after written informed consent. Cells remained stored in liquid nitrogen until use.

Human hybridoma generation. IgE-secreting human hybridomas were generated using methodology described previously (Wurth et al., 2018). Briefly, cryopreserved PBMCs obtained from subjects were expanded in culture at the approximate density of 2 million viable cells/plate into a 96-well flat bottom plates (Falcon, 353072) in the presence of 1 million gamma-irradiated NIH3T3 feeder cells genetically engineered to constitutively express human CD40L, IL-21 and BAFF (kindly provided by Dr. Deepta Bhattacharya; University of Arizona). Cells were incubated at 37° C. with 5% C02 for 6 days and then supernatants were screened for the presence of IgE using an optimized isotype-specific sandwich enzyme-linked immunosorbent assay (ELISA). Next, B cells were immortalized through electrical cytofusion with a non-secreting myeloma partner, HMMA2.5 myeloma cells (kindly provided by Marshall Posner), to produce an IgE-secreting hybridoma cell (Smith et al., 2012; 2105). To select for IgE-secreting hybridoma cells, fusion products were plated at 50 i1/well into 384-well plates (Nunc, 164688) containing hypoxanthine-aminopterin-thymidine (HAT) medium containing ouabain. Plates were incubated at 37° C. for 14 days before screening hybridomas for IgE antibody production by ELISA. Several rounds of limiting dilution plating and indexed single-cell flow cytometric sorting were employed to obtain a single clone of hybridoma. Once clonality was achieved, IgE mAbs were expressed in serum-free medium (Gibco Hybridoma-SFM; Invitrogen, 12045084). Immunoaffinity chromatography using the therapeutic mAb Omalizumab covalently coupled to GE Healthcare NHS activated HiTRAP (17-0717-01) was employed for the purification of IgE antibodies. Quantification of purified IgE mAbs was performed by UV spectrophotometry using a NanoDrop spectrophotometer.

Sequence analysis of antigen-reactive mAb sequences. Total RNA was extracted from 1 million clonal IgE-expressing human hybridoma cells using an RNeasy kit (Qiagen: 74104). Reverse transcription PCR (RT-PCR) was performed using the OneStep RT-PCR kit (Qiagen: 210210) with the 5′ primer set described previously (Smith et al., 2009) and a 3′ primer specific to the IgE constant region. cDNA products were purified and cloned into pCR2.1 using a TA cloning kit (Invitrogen: 45-0046). Antibody genes were Sanger-sequenced and analyzed using the IMGT database. Sixteen unique nucleotide sequences were analyzed for V/D/J gene usage, CDR3 length and somatic mutation.

Parasite material. Adult parasites, infective larvae (L3), L4 larvae and microfilariae of B. malayi were obtained from the NIAID/NIH Filariasis Research Reagent Repository Center (FR3; Athens, GA; www.filariasiscenter.org). Additional adult B. malayi were purchased from TRS Labs (Athens, GA). D. immitis worms were also a source of filarial antigens: adult male and female D. immitis parasites were obtained from surgical removal in severe cases of heartworm infection in canines. Soluble antigen from adult B. malayi and D. immitis worms was prepared by grinding frozen worms to a fine powder in liquid nitrogen using a mortar and pestle (cryogenic grinding). The homogenized powder was suspended in phosphate buffer saline (PBS 0.05 M, pH 7.2) containing Halt protease inhibitor cocktail (Thermo Scientific, 78429) rocking for 1 hr at 4° C. Proteins soluble in PBS were recovered by centrifugation (15,000 g) at 4° C. for 30 min. Protein concentration was measured by absorbance at UV280. Filarial worm extracts were aliquoted and stored at −20° C. until use.

ELISA binding assays. Wells of 384-well clear ELISA plates (Greiner, 781061) were coated with filarial worm extract or purified recombinant antigens (with estimated concentration of 10 gg/mL) at 4° C. overnight. Plates were blocked with 100 pl blocking solution/well (5% nonfat dry milk plus 2% goat serum in PBS) and incubated at room temperature for 1 hr. IgE mAbs (at an estimated concentration of 1-10 μg/mL) were added for a 1 hr incubation at room temperature. Bound antibodies were detected using an HRP-conjugated mouse anti-human IgE Fc secondary antibody (Southern biotech, 9160-05, 1:1000 dilution) and a 3,3′,5,5′-tetramethylbenzidine (TMB) substrate (Thermo Fisher Scientific). Color development was measured at 390 nm after 10 min using a Molecular Devices plate reader. An antibody was considered filaria-reactive if it bound to filarial extracts with an OD greater than three times background. For dose-response assays, antigen-coated plates were incubated with threefold serial dilutions of purified mAbs in blocking solution in triplicate, and antibody binding was detected as detailed above. Half maximal effective concentration (EC₅₀) values for binding were determined using Prism v.9.0 software (Graph-Pad) after log transformation of the mAb concentration using sigmoidal dose-response nonlinear regression analysis.

Immunoblotting. Filarial worm somatic extract or recombinant filarial antigens were mixed with loading buffer, separated by SDS-PAGE on 4-12% gradient gels (Thermo Scientific, NP0322BOX) with a prestained protein ladder (Thermo Scientific 26616) under nonreducing conditions. Antigen components were electrophoretically transferred to PVDF membranes (Thermo Scientific, LC2005) at 40 V for 150 min. After blocking with 5% nonfat dry milk in PBS overnight at 4° C., membranes were incubated with IgE mAbs (at a concentration of 1-10 μg/mL) or patient serum in blocking buffer for 1 hr at room temperature. Bound antibodies were probed with murine anti-human IgE peroxidase conjugate secondary antibody (Southern biotech, 9160-05) at 1:1000 dilution in blocking solution and incubated 1 hr at room temperature. Blots were washed 3 times with PBS between each step and visualized using chemiluminescent substrate (Supersignal Pico; Thermo Scientific, 34577) on an Amersham™ Imager 600.

ImmunoCAP Analysis. Prototype ImmunoCAP B. malayi tests were developed for research use from a B. malayi somatic extract. Analytical characteristics of ImmunoCAP tests were determined and an accelerated stability study was performed. The test was then used to screen reactivity of human IgE mAbs (at an approximate concentration of 1-10 μg/mL) against Brugia. An antibody was considered positive if it bound to B. malayi in ImmunoCAP (>1.0 kUA/L). The inventor notes that the assigned cutoff for ImmunoCAP positivity is much higher than the standard cutoff for serum analysis, as the inventor used excess IgE antibody and antigen concentration is the limiting factor.

Immunoprecipitation and mass spectroscopy. Human IgE mAbs that bound to B. malayi and/or D. immitis somatic extracts in ELISA, ImmunoCAP and/or immunoblot analysis were used for immunoprecipitation (IP). Each purified mAb was covalently coupled to magnetic microbeads (100 μg mAb per mg of beads) and incubated at 37° C. overnight on a rotating mixer, following the manufacturer's instructions (Invitrogen Dynabeads, 14311D). Antibody-coupled beads were washed several times to remove non-covalently bound antibody and then quenched. Next, filarial extracts were incubated with antibody-coupled beads for 30 min at room temperature. Beads were washed five times with PBS prior to elution of target protein using 1M glycine solution, pH 3.0. Each IP was performed in parallel with a control allergen-specific IgE mAb to monitor nonspecific interactions between filarial antigens and IgE antibodies. Eluted target antigen was separated on SDS-PAGE and stained with SimplyBlue SafeStain (Thermo Scientific, LC6060), and prominent bands of expected size were cut for mass spectrometry (MS) analysis, a step included to improve the signal to noise ratio in proteomics analysis. Due to poor annotation of genomes of filarial worms, an in-house protein database was developed for both D. immitis and B. malayi for use in analysis of MS data. RNAseq data used in D. immitis and B. malayi databases are available at GenBank under BioProject accession numbers PRJNA80937 and PRJNA344486, respectively.

Recombinant antigens and proteins. Recombinant His-tagged filarial antigens were expressed in bacteria. In brief, DNA sequences encoding WbSXP-1 (Acc number: AAC17637.1), Macrophage Migration Inhibitory Factor (MIF) (Acc number: EJW88743.1), 24 kDa secreted protein (Acc number: VI090327.1), Ladder antigen-like protein (GenBank: AAG31482.1), and Transthyretin-related proteins (GenBank: EJW82905.1 [TTR_05], EJW84161.1 [TTR_61], VDM07632.1 [TTR_32], EJW80404.1 [TTR_04], EJW78295.1 [TTR_95], EJW86262.1 [TTR_62], EJW85307.1 [TTR_07], VDM14847.1 [TTR_47], EJW86116.1 [TTR_16], EJW78979.1 [TTR_79], VDM13477.1 [TTR_77], VDM14190.1 [TTR_90], VDM14676.1 [TTR_76], VDM13708.1 [TTR_08], and VDM08841.1 [TTR_41]) were amplified from a synthetic gene construct (Genscript) and cloned into the expression vector pET-28a (with an N-terminal hexahistidine tag). Expression vectors were used to transform SHuffle® T7 Competent E. coli (NEB, C3029J), which are engineered to correctly fold disulfide bonded proteins in their cytoplasm (Lobstein et al., 2012). Transformed bacteria were grown at 37° C. in 1 liter of LB supplemented with 50 g/ml kanamycin with shaking at 220 rpm until they reached an OD (at 600 nm) of 0.5-0.8, and then protein expression was induced by addition of 1 mL 1 M IPTG (isopropyl thio-d-galacto pyranoside). Cultures were grown 20 hrs at 16° C. with shaking at 220 rpm. Harvested cells were pelleted by a 20 min centrifugation at 4000 rpm and then suspended in binding buffer (25 mM NaPi, 150 mM NaCl with pH=8) containing 1 μM PMSF, 0.7 μg/ml pepstatin, 1 μg/ml leupeptin, DNase, Lysozyme. An Avesti EmulsiFlex C3 benchtop homogenizer was used to disrupt cells and obtain a somatic extract. Insoluble material was removed by centrifugation at 12,000 rpm for 20 min at room temperature. Recombinant protein was purified using TALON metal affinity resin (Takara, 635504) following the manufacturer's specifications. Briefly, 5 mL TALON resin was added to lysates, which were rocked 1 hr at 4° C. The slurry was then suspended in a glass column for gravity chromatography. The resin was washed with 10 column volumes of washing buffer (25 mM NaPi, 150 mM NaCl+20 mM imidazole with pH=7) and the protein eluted with 5 column volumes of washing buffer containing imidazole at 500 mM. Protein concentration was estimated by the method of Lowry et al. (1951). Finally, Zeba Spin Desalting Columns (Thermo Scientific, 89893) were used to remove imidazole and to exchange buffer to PBS. Purified proteins were assessed for purity and appropriate molecular weight by SDS-PAGE.

Phylogeny of WbTTR proteins. Amino acid sequences of all TTR-52 domain-containing proteins reported for W. bancrofti were derived from the National Center for Biotechnology Information database (NCBI) using TTR52 domains (pfam01060) and filtering for W. bancrofti sequences. Phylogenetic analysis and phylogeny estimation were performed using the web-based software tool MAB (Methods and Algorithms for Bioinformatics), and a maximum-likelihood phylogenetic tree was constructed using the MUSCLE algorithm. Partial and repetitive sequences reported in NCBI were not included.

Analysis of cross-reactivity of TTR-specific antibodies. ELISAs were performed to semi-quantitatively assess binding of TTR-specific mAbs to different TTR proteins. Recombinant TTR proteins were coated to ELISA plates overnight at 4° C. at a minimum concentration of 10 μg/mL. After blocking, 25 μL of primary antibody at 2 ng/μL was incubated 1 hour in blocking buffer. Bound antibodies were detected using HRP-conjugated mouse anti-human IgE Fc secondary antibody (Southern biotech, 9160-05, 1:1000 dilution) and 3,3′,5,5′-tetramethylbenzidine (TMB) substrate (Thermo Fisher Scientific). Color development was measured at 390 nm after 10 min using a Molecular Devices plate reader. OD values are reported after subtraction of signals from wells containing blocking buffer only. The 12D4 mAb served as negative control.

Human FcεRI-transgenic mouse anaphylaxis. Mouse studies were carried out in accordance with recommendations in the Guide for the Care and Use of Laboratory Animals of the National Institutes of Health. Human FcεRI-transgenic mice [B6.Cg-Fcεr1α^{tm1Knt}Tg(FCERIA)1Bhk/J]were purchased from The Jackson Laboratory (stock 010506), bred and genotyped. These mice carry 2 gene mutations: the human Fc fragment of IgE receptor a polypeptide (FCER1A) under control of the human FCER1A promoter and a mutation targeting Fcεr1α^{tmIK}at blocking expression of murine FCER1A⁴¹. Human IgE can induce anaphylaxis in mice hemizygous for the transgene and homozygous for targeted deletion of mouse FcR1. Transgenic mice were sensitized by i.p. injection of 100 μg total IgE and challenged by i.p. injection of 50 g purified recombinant antigen. Changes in mouse core body temperature were monitored over 90 min using implanted temperature probes.

Statistics. For ELISA studies, mean relative fluorescence units were calculated using three independently performed assays performed in triplicate. The descriptive statistics mean±SEM or mean±SD were determined for continuous variables as noted. Statistical analysis was performed using Prism v9.0 (GraphPad) software. In mouse studies, the comparison of temperature-change curves was performed independently for each antigen challenge and at each time point using paired 2-tailed t test, assuming unequal variance (Microsoft Excel Office Professional Plus 2016). Time points with calculated P values less than 0.05 were labeled and considered significant. Error bars for mouse temperature measurements represent SEM.

### Example 2—Results

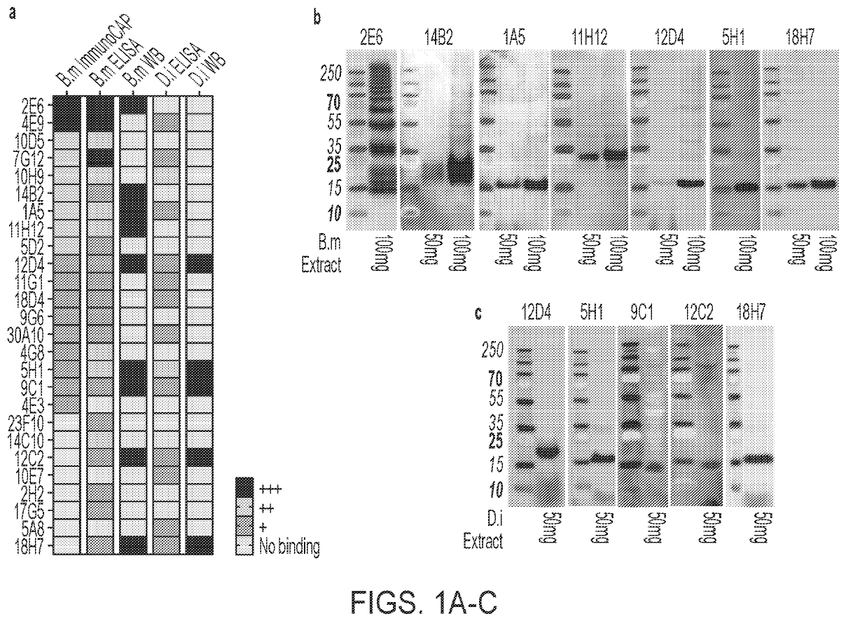

IgE-Positive B Cells are Present in Blood of Subjects with Filariasis. To date, few studies have directly examined the B cells that produce IgE antibodies in humans (Wurth et al., 2018; Croote et al., 2018). Thus, the inventor asked whether IgE-producing B cells are present in blood of subjects infected with filarial nematodes and, if so, could they be used to generate human IgE mAbs. To do so, the inventor obtained frozen peripheral blood mononuclear cells (PBMC) from subjects with lymphatic filariasis, TPE, loiasis, or onchocerciasis. Using previously published human hybridoma methodology (Wurth et al., 2018) (Extended Data FIG. 1a), the inventor generated 56 IgE mAbs from B cells from 7 individuals without selection for antigenic specificity (Supplementary Table 1). Those observations indicate a frequency of IgE-expressing B cells in circulation of these subjects ranging from 6-14 cells per 10 million PBMCs, slightly lower than the number of IgE-expressing B cells the inventor previously reported for subjects with allergic bronchopulmonary aspergillosis (Wurth et al., 2018).

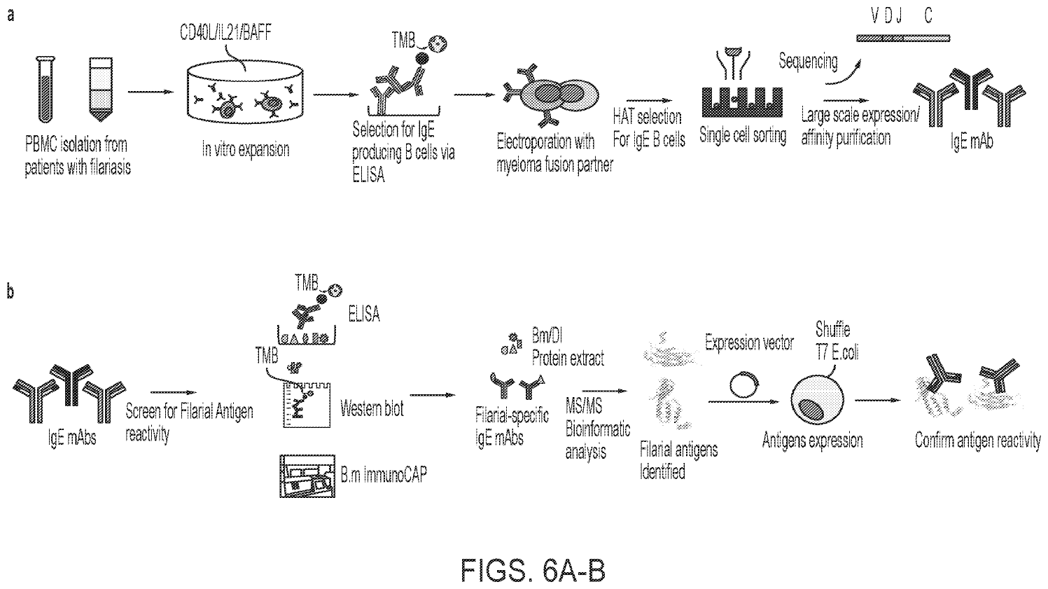

Identification of Filaria-Specific IgE Antibodies. The inventor next determined whether candidate IgE mAbs were directed against filarial antigens (FIG. 6b). To do so the inventor produced whole-worm sonatic extracts from B. malayi or Dirofilaria immitis, which are responsible for filariasis in humans or dogs, respectively, and tested mAb reactivity against each extract using three complementary assays - immunoblotting, ELISA and B. malayi ImmunoCAP. Of the 56 human mAb candidates, 26 were positive in at least one screen and some in all three (FIG. 1a and Supplementary Table 2). Of the 26 antibodies with detectable binding, 9 (35%) were immunoblot-positive against B. malayi with 5 (19%) also capable of blotting D. immitis. Representative blot images showing different banding patterns observed for candidate mAbs are shown for B. malayi in FIG. 1b and D. immitis in FIG. 1c. MAb 2E6, which had the greatest positivity in B. malayi ImmunoCAP, also showed robust positivity on immunoblots and exhibited a ladder-like pattern ranging from −14 kDa to >250 kDa. Interestingly, except for mAb 2E6, all bands detected by immunoblot analysis were <30 kDa in size.

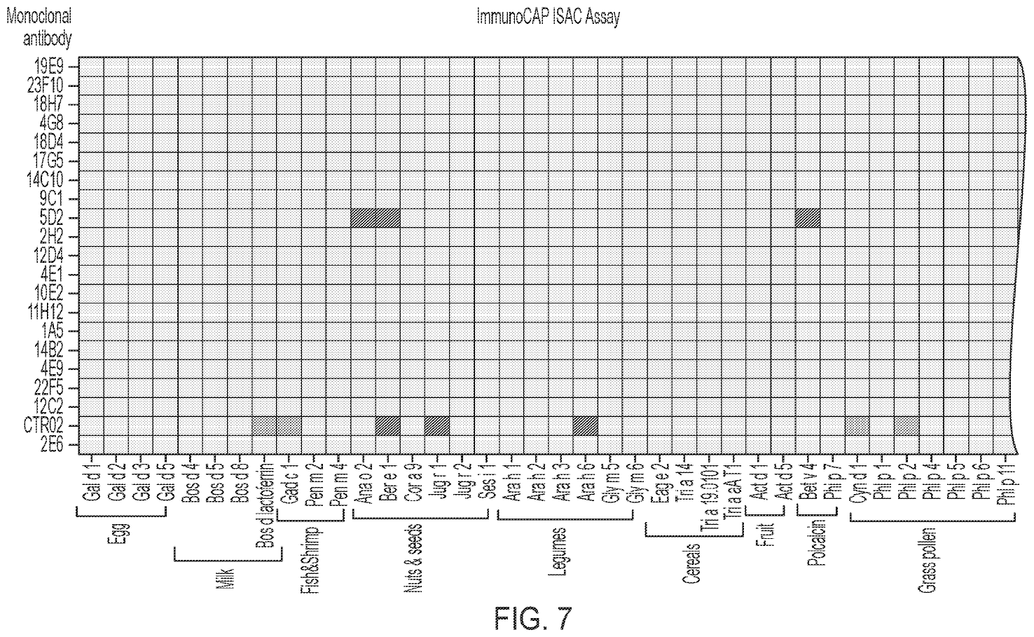

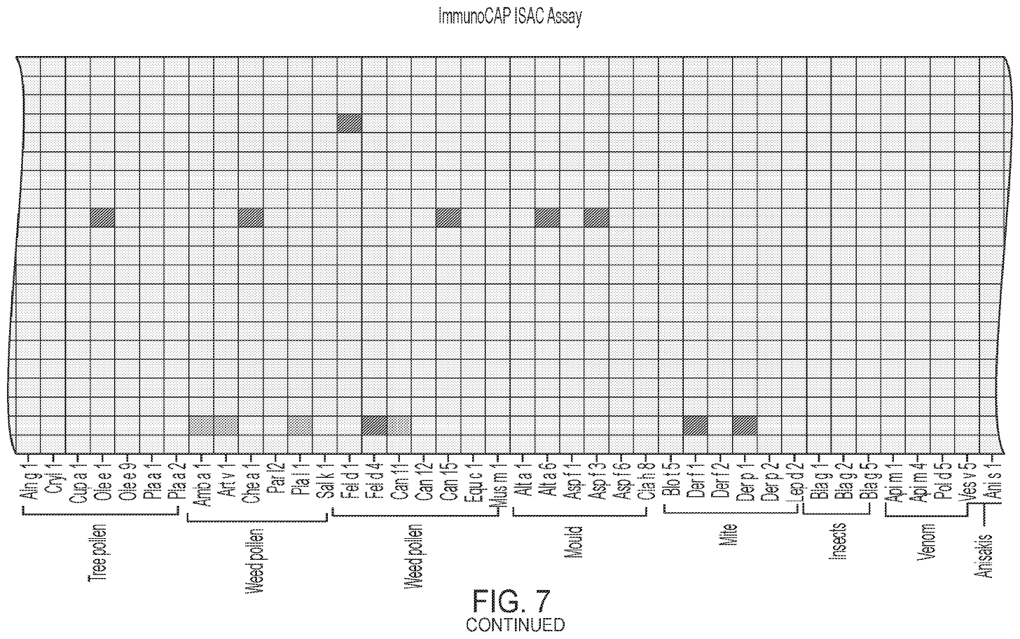

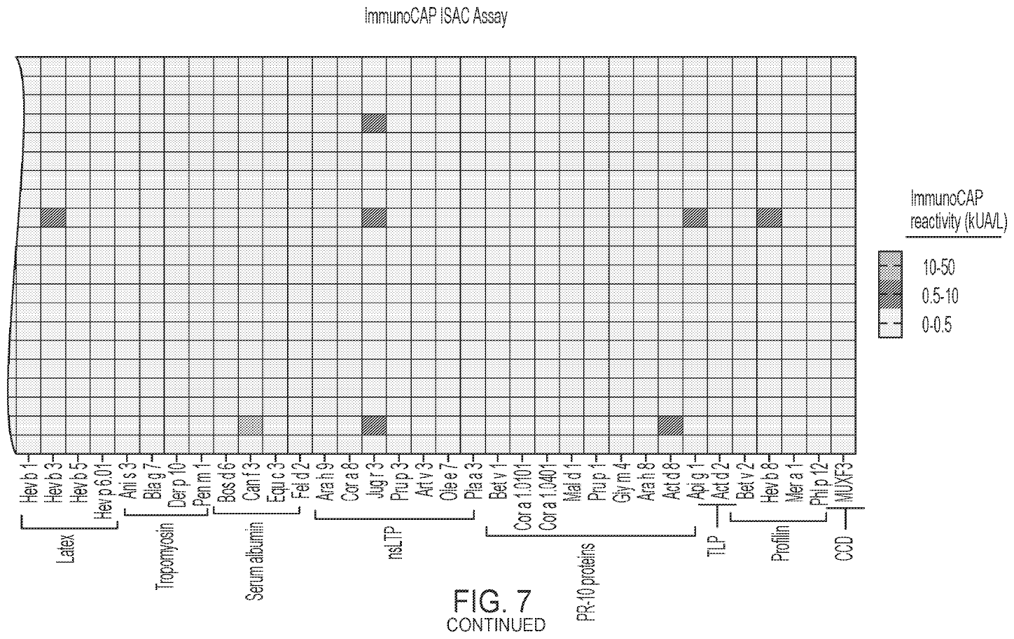

Given reports that IgE from patients with parasitic infection cross-reacts with common allergens (Gazzinelli-Guimaraes et al., 2021; Santiago et al., 2011), the inventor asked whether his 26 candidate antibodies obtained from filaria-infected subjects cross-reacted with common allergen proteins. To do so the inventor analyzed all 26 using an ImmunoCAP ISAC allergen microarray containing 112 purified allergens. The inventor observed no significant reactivity to any antibody tested (FIG. 7), suggesting that the IgE antibody response induced during filarial infection are highly filarial-specific.

Of note, of the 26 filaria-specific IgE mAbs, 15 were obtained from the two subjects with TPE (15 of their 23 mAbs, 65%). Because W. bancrofti (which is responsible for 90% of LF infections), Loa loa and Onchocerca volvulus cannot be maintained in conventional mouse strains, the inventor could not screen IgE mAbs against these filarial somatic extracts and thus could not identify Wuchereria-specific, Onchocerca-specific, or Loa-specific IgE mAbs in this panel. Of the 26 filaria-specific IgE mAbs, only 13 showed cross-reactivity towards dog heartworm (Supplementary Table 2). Taken together this suggests that the IgE antibody response is highly specific to antigens of the infecting filarial worm, though, at the protein level there is a high (>90%) sequence identity across the filarial species that are pathogenic for humans.

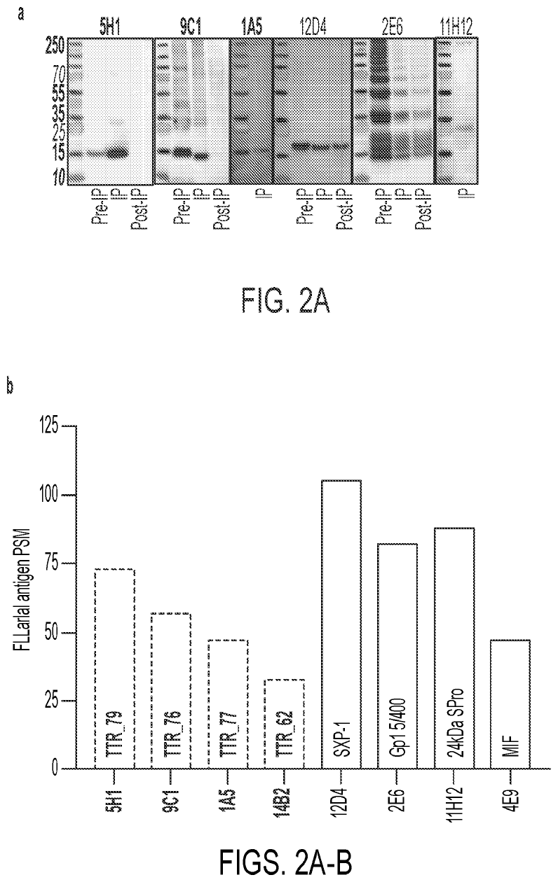

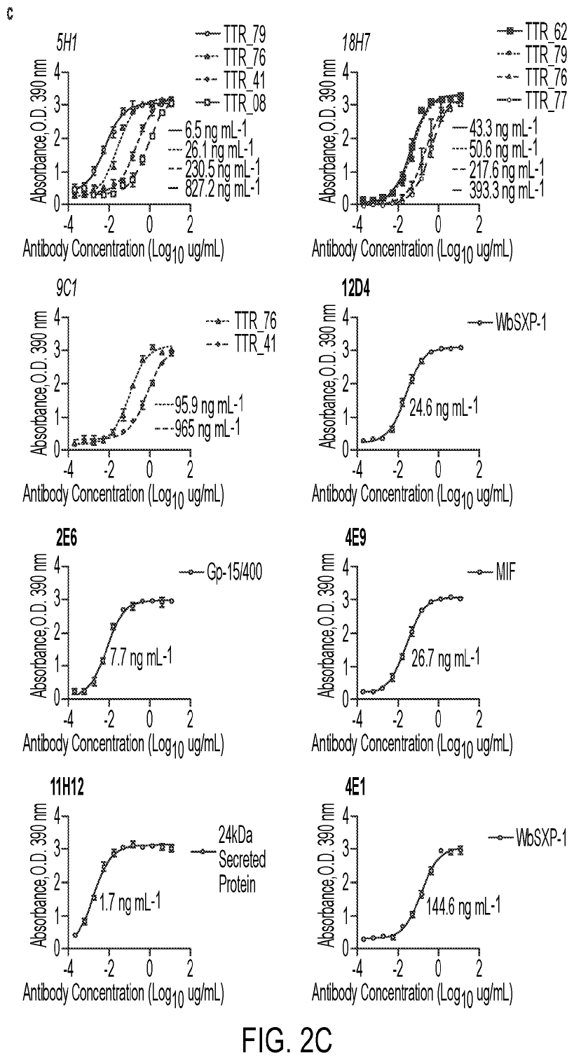

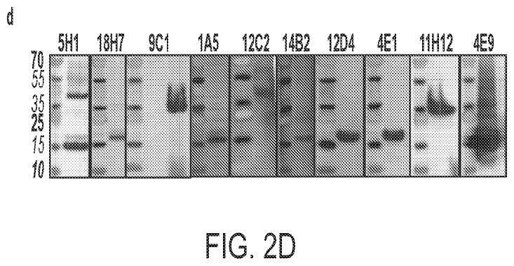

The Antigenic Landscape of the Anti-filaria IgE Antibody Response. To assess antibody specificity, the inventor focused on the 26 antibodies that showed reactivity to a filarial worm extract in multiple assays, as described above. To identify antigen targets, the inventor independently immobilized the 26 candidate mAbs to magnetic beads and performed immunoprecipitation (IP) of B. malayi and D. immitis somatic extracts and subsequent mass spectrometry analysis. Immunoprecipitates with human IgE mAbs specific for irrelevant antigens from mold and peanut (mAbs 21E2, 16A8) served as specificity controls. IP was followed by immunoblotting to confirm antigen enrichment, as can be seen by examples shown in FIG. 2a. In some cases, such as with mAb 5H1 and 9C1, filarial antigen was at such a low concentration that IP resulted in complete depletion of antigen. In other cases, such as with mAb 2E6, a very high concentration of the filarial antigen resulted in saturation, as antigen can be seen in the post-IP material.

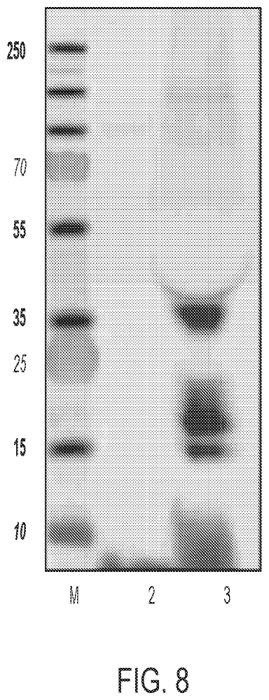

Overall, 16 IgE mAbs were able to immunoprecipitate a filarial antigen; the inventor identified 14 unique antigens (Supplemental Table 3). MAbs 1A5, 5111, 9C1 and 14B2 precipitated transthyretin-related protein (FIG. 2b), a 16 kDa nematode-secreted protein and member of the “transthyretin-like” family of proteins. Proteins of this family are reportedly prominent components of the secretome of several parasites including B. malayi (Hewitson et al., 2008), D. immitis (Geary et al., 2012), T. colubriformis (Cantacessi et al., 201), and C. elegans (Sonnhammer & Durbin, 1997). Their function is largely unknown, but a TTR protein from the plant parasite M. javanica reportedly interferes with the host immune response and promotes parasitism (Lin et al., 2016). MAb 12D4 precipitated WbSXP-1 (FIG. 2b), a 14 kDa nematode-specific secreted protein (also known as Wbl4 antigen) previously reported as a target of IgG4 antibody (Dissanayake et al., 1992) and an antigen used in multiple diagnostic tests (Rao et al., 2000; Rahman et al., 2007). WbSXP-1 homologues are present in Anisakis (Garcia-Mayoral et al., 2014) and have been pursued as vaccine candidates against Ascaris (Naotoshi et al., 2003). MAb 11H12 precipitated a 24 kDa nematode-specific secreted protein with no known function (24 kDa SPro) (FIG. 2b). Although there were no NCBI sequences reported for the W bancrofti homologue of this protein, a similar protein in D. immitis named 22-24 kDa excretory-secretory 22U protein has been reported (Frank et al., 1999). MAb 4E9 precipitated Macrophage Migration Inhibitory Factor (MIF) (FIG. 2b), a worm homologue of human MIF, a proinflammatory cytokine (Pastrana et al., 1998). Filarial MIF functions in immunoregulation and possibly pathogenesis via attracting host monocytes/macrophages to modify host immune responses and promote parasite survival. Finally, mAb 2E6, which showed the most intense signal in all screening assays, precipitated the polyprotein ladder-like protein or gpl5/400 (FIGS. 1a, 1b, 2a), a ˜400 kDa nematode polyprotein allergen/antigen (NPA-1) with 20 tandemly arranged repeat subunits of 132 amino acid residues. B. malayi gpl5/400 protein is associated with the worm surface and distributed in all tissues of the parasite (Tweedie et al., 1993). The presence of gpl5/400 protein in the B. malayi secretome was confirmed by immunoblot of culture media used to keep worms alive for 7 days (FIG. 8). While antibody 2E6 is specific for B. malayi and W bancrofti gp15/400, it did not show cross-reactivity with D. immitis gpl5/400.

To confirm IgE mAb specificity, the inventor expressed recombinant forms of 19 filarial antigens (SXP, 24 kDa SPro, MIF, gpl5/400, and 15 different TTR proteins) in bacteria using the W. bancrofti sequence and tested binding by ELISA (FIG. 2c). If IgE mAbs showed positivity in an immunoblot of somaic extract the inventor also performed immunoblotting (FIG. 2d). When the inventor was able to express and purify antigen protein in sufficient quantities, the inventor calculated the EC₅o for each antibody (FIG. 2c, Supplementary Table 3). EC₅o measurements ranged from single ng/mL, such as with mAb 11H12, to nearly g/mL concentrations. Antibodies 5H1 and 18H7 showed a range of EC₅o binding values to different TTR proteins, demonstrating their varied breadth of cross-reactivity within this protein family (FIG. 2c). Another interesting feature of TTR protein-specific mAbs is their varied tendency to bind TTR dimers (see FIG. 2d). MAb 9C1 binds preferentially to dimeric TTR, 18H7 prefers monomeric TTR, and 5H1 binds equally to both.

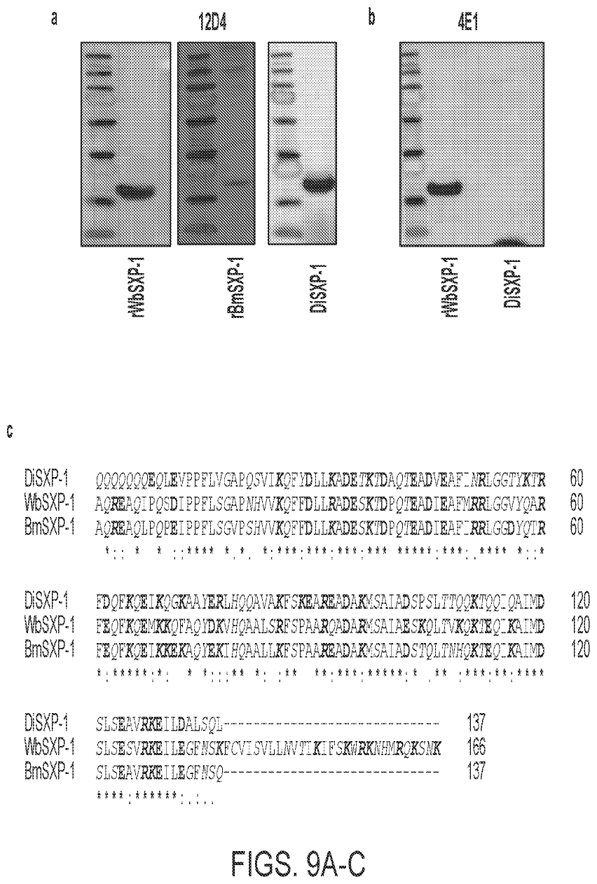

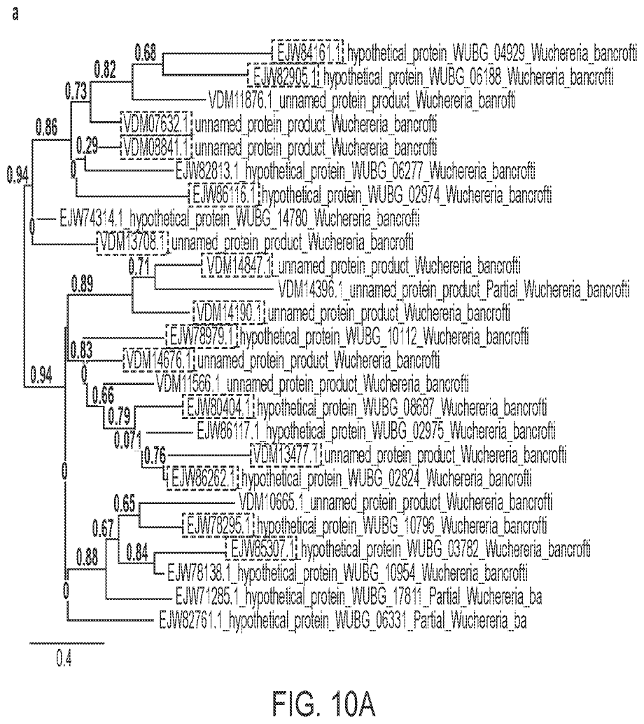

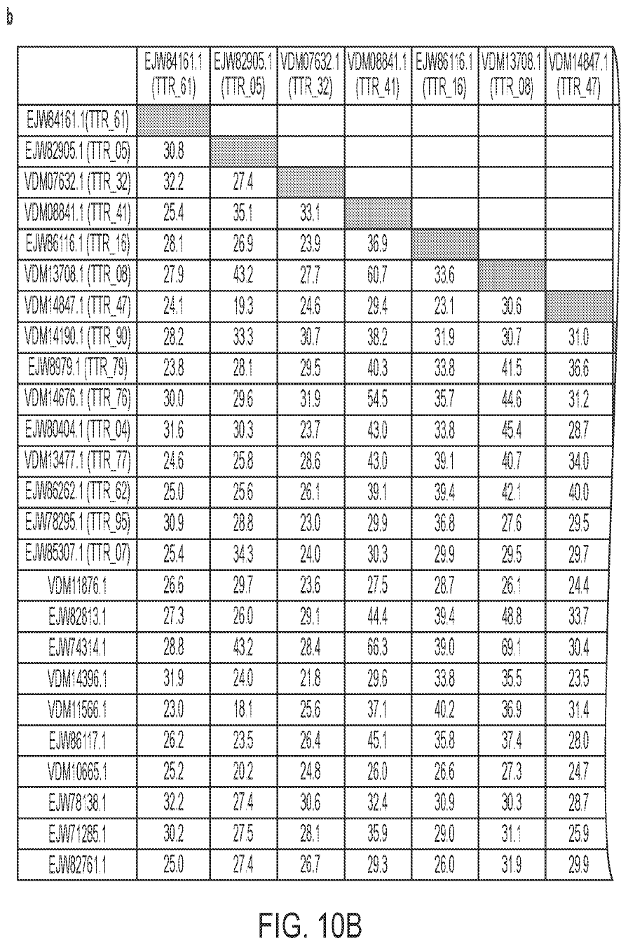

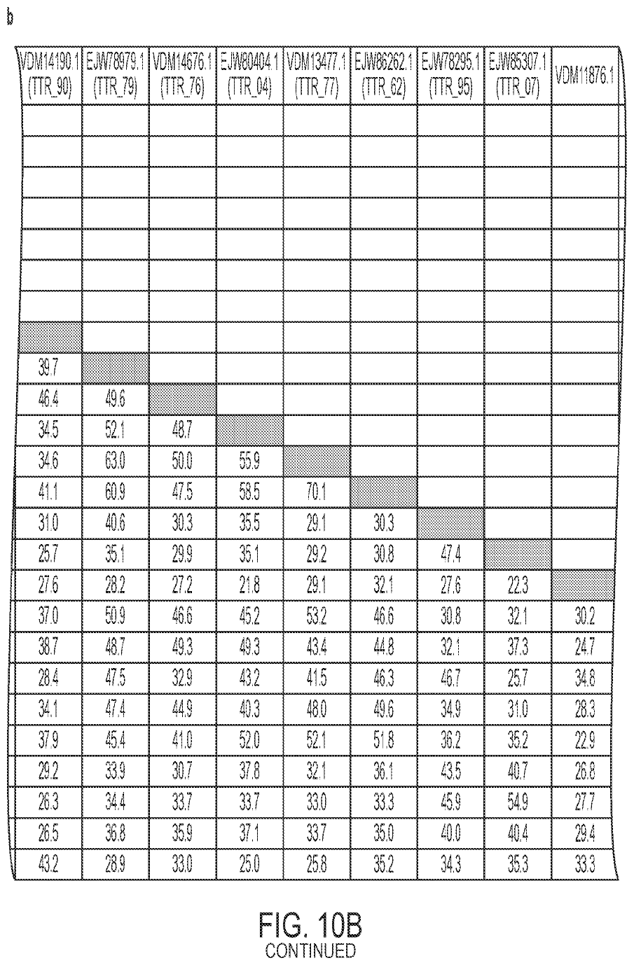

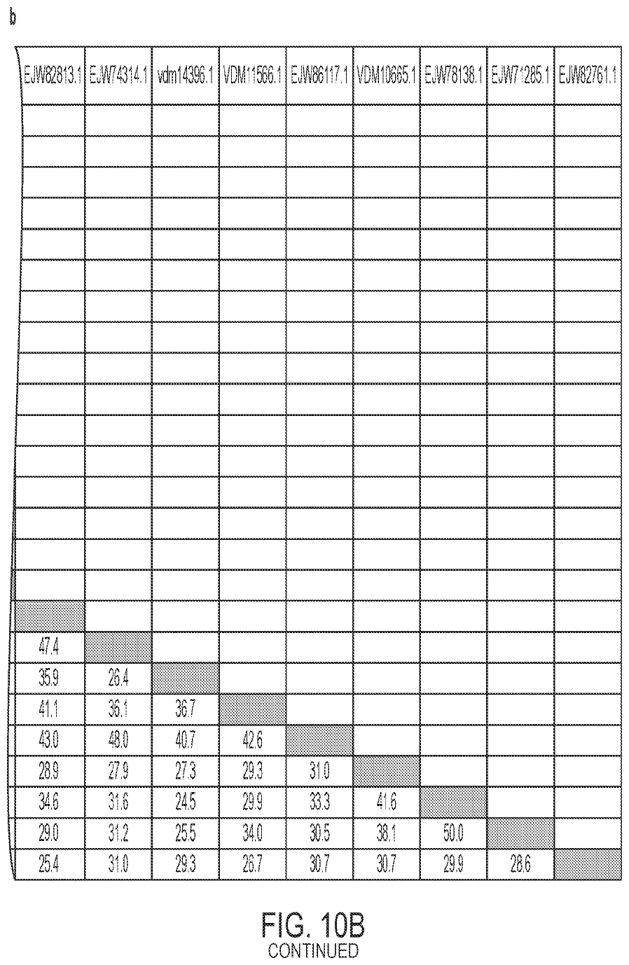

The inventor next tested the 30 IgE mAbs from the original 56 that had not tested positive in reactivity tests against each recombinant antigen to ensure that no antibody was missed due to its specificity for W. bancrofti. MAb 4E1, for example, was found specific for WbSXP-1 but negative in all primary extract screens. Unlike mAb 12D4, 4E1 is highly specific to W bancrofri and does not cross-react with B. malayi or D. immitis SXP-1 proteins (FIGS. 9a-c). Finally, two mAbs, 5D2 and 7G12, were positive by ELISA and ImmunoCAP but negative in IP analysis (Supplemental Table 2). MAb 5D2 showed weak binding to several allergen proteins based on the immunoCAP ISAC assay (FIG. 7), suggesting poly-reactivity to a carbohydrate epitope. Overall, these results indicate that the humoral immune response to filarial infection targets a restricted set of filarial-specific E/S proteins. TTR Proteins are Immunodominant Filarial Antigens with Diagnostic Potential.

TTR proteins identified here as target antigens of multiple filaria-specific IgE mAbs have not been previously reported as filarial immunogens. Thus, the inventor asked whether the inventor's panel of filaria-specific IgE included mAbs specific to other TTR proteins. To do so, the inventor first assembled a phylogenetic tree using WbTTR protein sequences collected from NCBI (FIG. 10a). TTR family proteins shared a low degree of identity, ranging from 18.1% to 70.1% when compared in pairwise fashion (FIG. 10b).

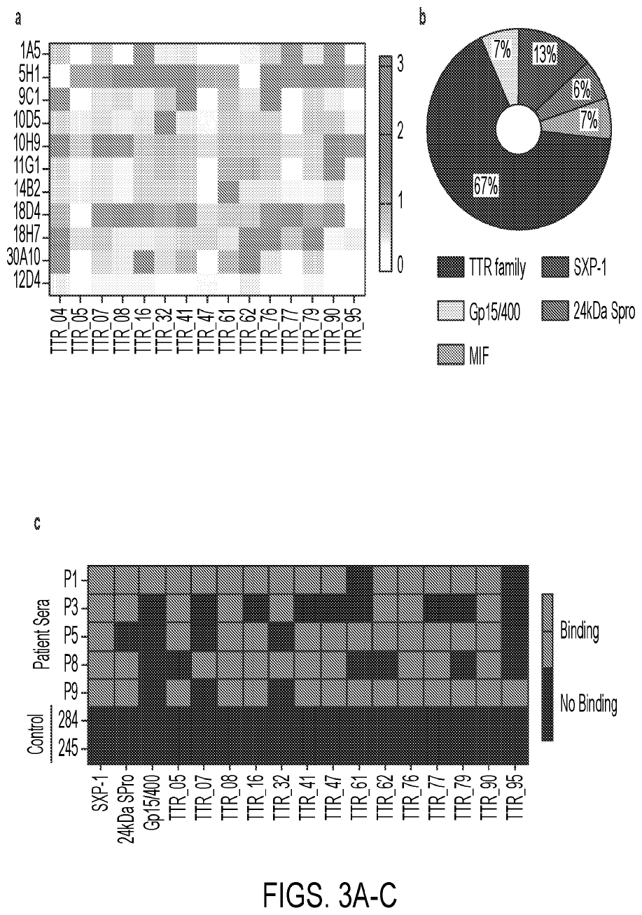

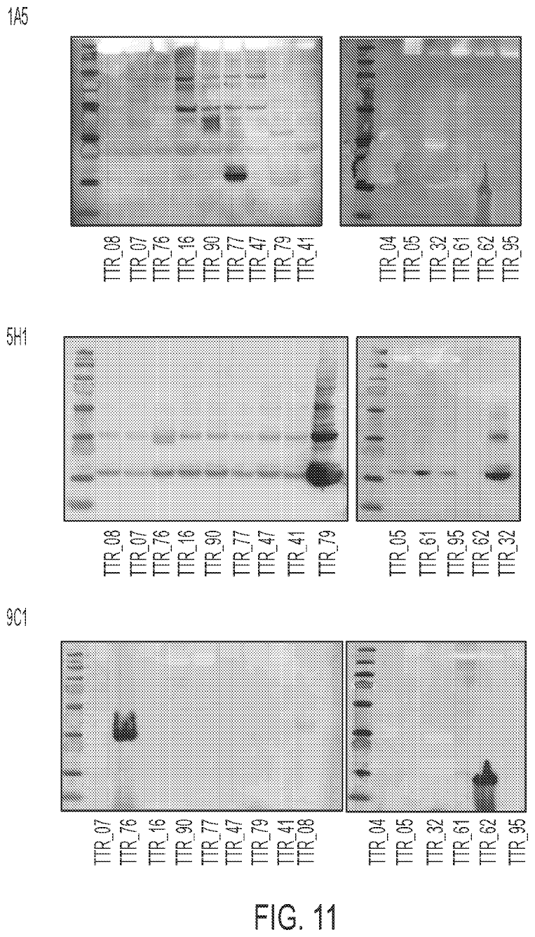

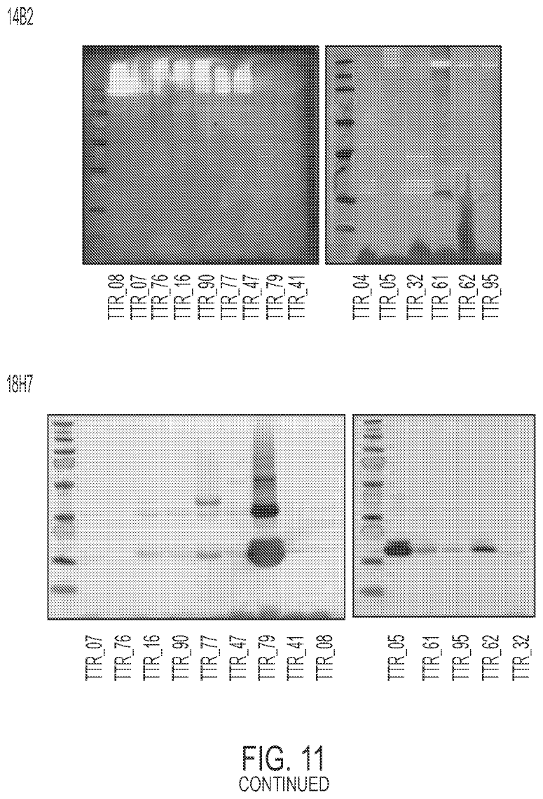

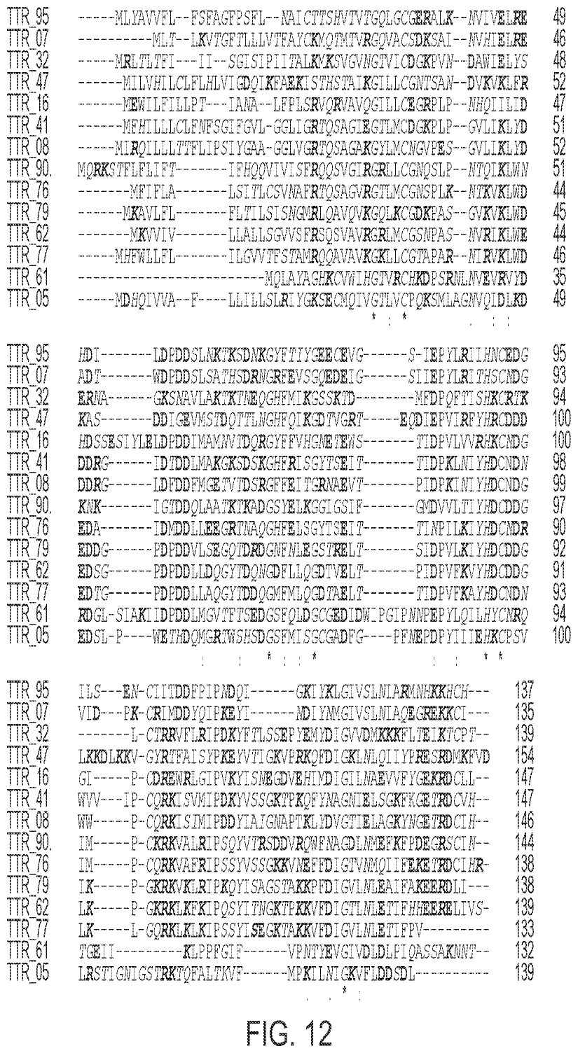

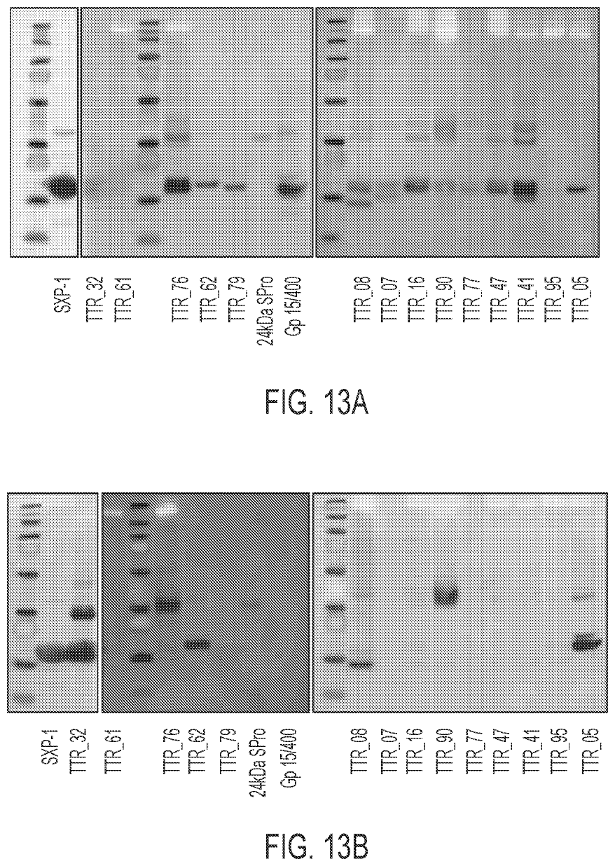

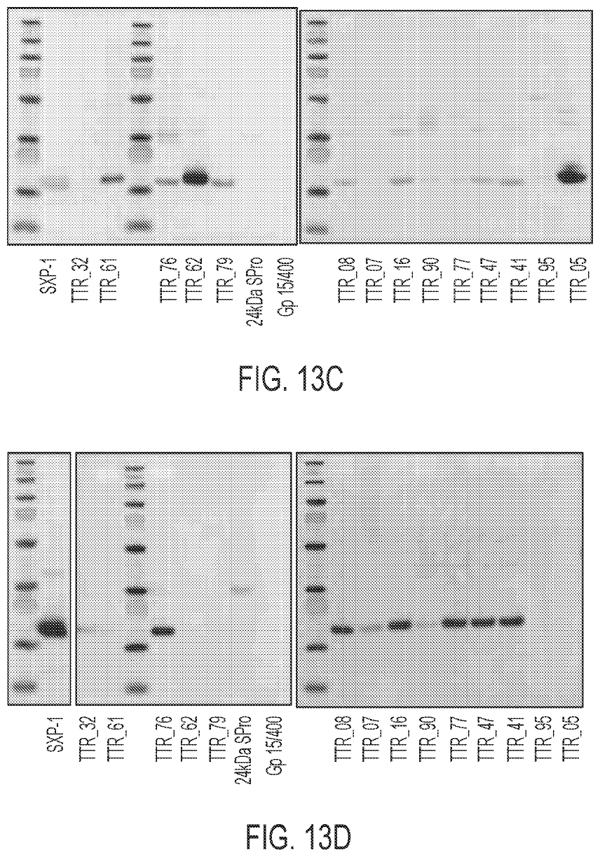

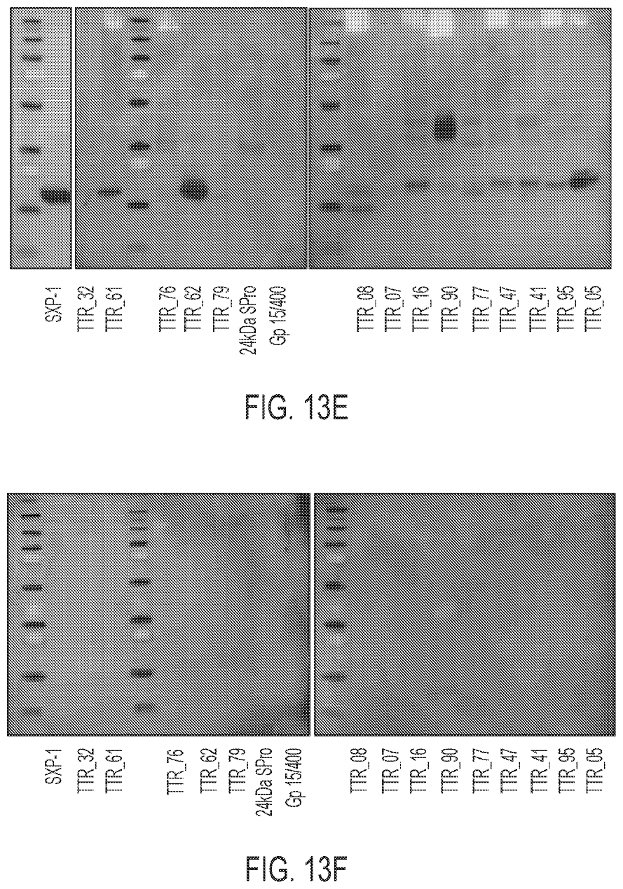

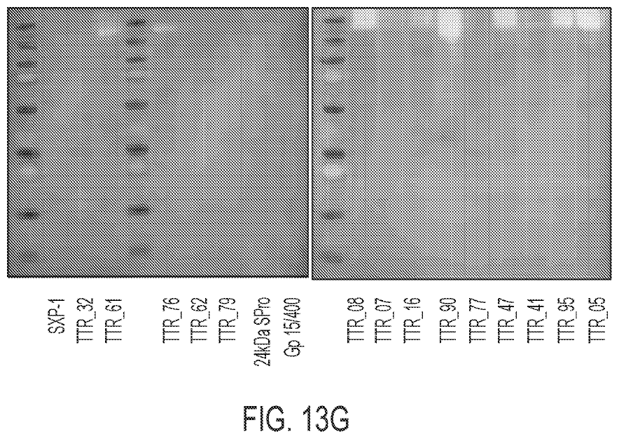

Next, the inventor chose 15 TTR proteins as representatives of the family and expressed each as a His-tagged fusion protein for IgE mAb testing in ELISA and immunoblotting. Binding analysis of the 15 TTR proteins with 10 IgE mAbs revealed that some antibodies were highly specific to one TTR protein (FIG. 3a), while others showed varying degrees of cross-reactivity across different TTR protein family members (FIG. 3a, FIG. 11). Of note, two IgE mAbs, 5H1 and 18H7, broadly cross-reacted with 13 different TTR proteins despite minimal amino acid conservation (FIG. 2c, FIG. 3a, FIGS. 10b, 11, 12). Antibodies targeting TTR protein antigens made up the greatest proportion of the inventor's panel of filaria-specific antibodies (FIG. 3b). Overall, these results indicate that TTR family proteins are the most dominant filarial antigens targeted by the human IgE antibody response (FIG. 3b, Supplementary Table 3). The inventor next tested sera of subjects with filarial infection for the presence of filaria-specific IgE antibodies using immunoblotting of 17 recombinant filarial antigens. The inventor chose 3 subjects (P1, P3, P5) from whom IgE mAbs were generated from their PBMCs in this study, and 2 subjects (P8, P9) in which no PBMCs were used and no IgE mAbs were developed, to assess each for the presence of specific antibodies. Immunoblotting not only verified the presence of antibodies with the same specificity as those of the inventor's IgE mAbs in the serum of subjects from whom mAbs were developed, but also confirmed presence of antibodies with similar specificities in other filarial worm infected subjects. Each subject sample showed a distinct pattern of reactivity predominantly to TTR family proteins (FIG. 3c, FIGS. 13a-g), confirming that antigens identified here could be used to develop species-specific diagnostics.

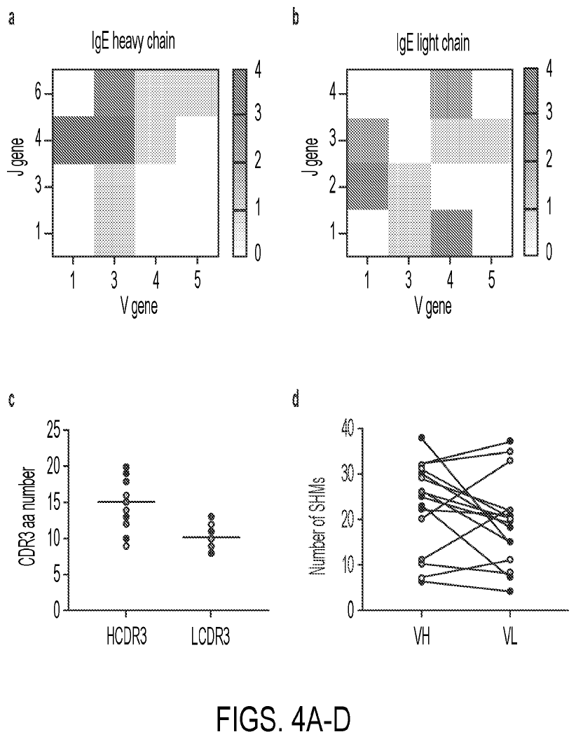

Filaria-Specific IgE mAbs Exhibit a High Degree of Somatic Hypermutation (SHM). To evaluate sequence diversity and degree of somatic hypermutation, the inventor performed sequence analysis of 16 filaria-specific IgE hybridoma clones (FIGS. 4a-d). That analysis revealed 16 unique Vh-Jh-CDRH3-V1-JI-CDRL3 clonotypes (Supplementary Table 4). Filaria-specific IgE antibodies varied widely in their antibody variable region (V_{H} and V_{L}) germline gene usage, with no statistically significant over-representation of any germline gene (FIG. 4a). They also varied in the lengths of V_{H} and V_{L} complementarity-determining region 3 (CDR3), but relative to other isotypes, IgE B cells show similar length distributions of CDR3 amino acids in the heavy and light chains (Soto et al., 2019) (FIGS. 4b-c). Filaria-specific IgE mAbs exhibited a high degree of SHM in V_{H} and V_{L} genes (FIG. 4d), which contained an average of 23 and 19 SHMs, respectively. No meaningful correlation was observed between V_{H} and V_{L} mutation frequencies. The high degree of SHM of filaria-specific antibodies suggests a prolonged and ongoing humoral immune response to antigens.

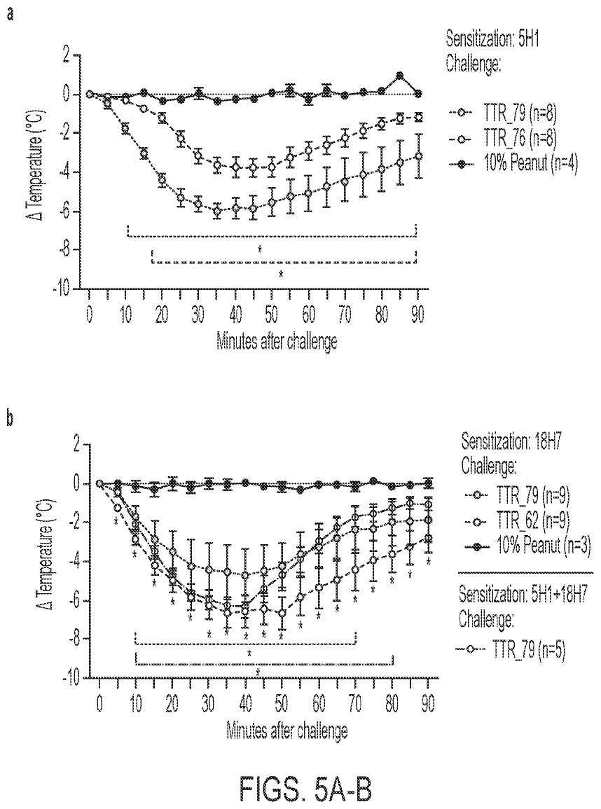

Filaria-Specific IgE Antibodies Induce Anaphylaxis in Mice. The inventor next assessed function of two filaria-specific IgE mAbs, 5H1 and 18H7, using a human FcR1α-transgenic mouse model of passive systemic anaphylaxis. The inventor chose these two IgE mAbs because of their ability to bind TTR protein dimers, which could in theory allow cross-linking of FcR1 to trigger mast cell degranulation. Thus, the inventor first sensitized mice with TTR-specific IgE mAbs 5H1, 18H7, or both, or with injection of an isotype control antibody. Three days later mice were challenged with single intraperitoneal injection with recombinant TTR proteins (50 μg of filarial antigen TTR_76, TTR_79, or TTR_62) or with control peanut allergen extract. All mice injected with recombinant TTRs, whether sensitized with mAbs 5H1, 18H7, or both, exhibited a significant drop in temperature of up to 6° C., reaching the nadir within 35 minutes of challenge, while animals receiving peanut allergen extract showed no temperature change (FIGS. 5a-b). This response is indicative of anaphylaxis (Osterfeld et al., 2010). Of note, the inventor observed no additive or synergistic effects in mice injected with both 5H1 and 18H7 IgE mAbs (FIG. 5b).

Despite a deep understanding of the major allergen proteins responsible for induction of allergic diseases, little is known about immunogenic triggers of the IgE antibody response in the defense against helminth infection. The inventor adopted an unbiased approach to investigate specificities and functions of IgE antibodies produced in the context of filarial worm infection. He reports that all filarial antigens targeted by IgE antibodies in this analysis were nematode-specific and E/S proteins. Since helminths do not proliferate within their human host, these observations suggest that evolutionarily the IgE antibody response developed to enable prompt recognition of invading worms at the larval stage as they penetrate the epithelial barrier. In addition, in vivo data shows that filaria-specific IgE antibodies induce a robust type 1 hypersensitivity response. Taken together, the inventor propose that IgE antibodies produced in response to parasitic infections function to prevent establishment of new infections at the epithelial site of entry. Mast cell activation occurring through IgE cross-linking associated with excreted worm antigens allows immediate detection, prompting eosinophil and basophil infiltration and targeting the worm when it is most vulnerable. Given the impact of helminthic disease in developing countries, it is of central importance to identify key helminth immunogens that can inform work toward improved diagnostics and the host/parasite interface. In addition to providing insights into the ontogeny of the IgE response in helminth infection, findings reported here have implications for rational design of helminth vaccines providing IgE-mediated pathology can be circumvented.

SUPPLEMENTARY TABLE 1
Subject demographics and IgE encoding B cell frequencies B-cell
Table S1. Human subject demographics. Subject helminth disease, serum IgE titer,
IgE B-cell frequency and IgE hybridomas yield are shown. IgE B cell frequencies
are expressed as the number of IgE-positive cells per 10 million PBMCs. The total
number of IgE-expressing human hybridomas generated for each subject is listed. No
PBMCs, only sera, were used from subjects P8 & P9. TPE, tropical pulmonary eosinophilia.

| | | | | Geographic | | IgE B-cell | IgE |
|---|---|---|---|---|---|---|---|
| Subject | | | | location of | Serum IgE | frequency (per | hybridomas |
| code | Age | Sex | Diagnosis | infection | IU/ml | 10⁷ PBMCs) | generated |
| P1 | 61 | M | TPE | India | 4260 | 13.5 | 22 |
| P2 | 29 | M | TPE | India | 9810 | 8.1 | 1 |
| P3 | 21 | F | Loiasis | Cameroon | 20290 | 9.2 | 9 |
| P4 | 40 | M | Loiasis | Cameroon | 8520 | 10.9 | 12 |
| P5 | 61 | F | Loiasis | Cameroon | 11362 | 6.2 | 5 |
| P6 | 29 | M | Loiasis | Gabon | 7628 | 6.0 | 1 |
| P7 | 42 | M | Onchocerciasis | Sierra Leone | 8680 | 13.7 | 6 |
| P8 | 49 | F | TPE | India | 6140 | Serum only | Serum only |
| P9 | 27 | M | TPE | India | 7440 | Serum only | Serum only |

SUPPLEMENTARY TABLE 2
IgE hybridoma reactivities
Table S2. Reactivity of IgE mAbs against B. malayi and D. Immitis. All IgE mAbs
were tested against B. malayi and D. immitis in ELISA and WB, as well as B. malayi
ImmunoCAP. IgE mAb binding to B. malayi and D. Immitis somatic extracts in ELISA
are represented as follows: − no binding detected; + binding 2-10 times background; ++
binding 10-100 times background; +++ binding >100 times background. IgE mAb
binding to B. malayi ImmunoCAP was considered positive if >1 kUA/L. IgE mAb binding
to B. malayi and D. Immitis somatic extracts in WB are reported as follows: − no
binding detected; +++ presence of a clear band. If binding was detected in any assay,
IP of the target antigen was attempted from crude worm somatic extracts and was followed
by mass spectrometry for identification. A target antigen was considered positive
if representing >10 times that precipitated by a control IgE mAb. N/A, IP not attempted;
ND, IP attempted but unsuccessful; kUA, allergen-specific kilo-units of antibody.

| | | B. malayi | | | | | |
|---|---|---|---|---|---|---|---|
| | | ImmunoCAP | Binding to B. | | Binding to D. | | |
| Subject | IgE | reactivity | malayi extract | | immitis extract | | |
| code | hybridoma | (kUA/L) | ELISA | Immunoblot | ELISA | Immunoblot | IP |
| P1 | 1A5 | 14.2 | ++ | +++ | + | − | + |
| | 10H9 | 28.6 | ++ | − | ++ | − | N/A |
| | 11H12 | 12 | ++ | +++ | − | − | + |
| | 14B2 | 23.9 | + | +++ | − | − | + |
| | 7G12 | 29.3 | +++ | − | + | − | ND |
| | 19E9 | 0.2 | − | − | − | − | N/A |
| | 23F10 | 0.5 | + | − | − | − | N/A |
| | 18H7 | 0.0 | + | +++ | + | +++ | N/A |
| | 4G8 | 2.2 | ++ | − | − | − | N/A |
| | 18D4 | 3.5 | + | − | + | − | N/A |
| | 17G5 | 0.1 | + | − | − | − | N/A |
| | 14C10 | 0.5 | ++ | − | − | − | N/A |
| | 22F5 | 0.0 | − | − | − | − | N/A |
| | 2E6 | 465.7 | +++ | +++ | − | − | + |
| | 15C1 | 0.1 | − | − | − | − | N/A |
| | 8A7 | 0.2 | − | − | − | − | N/A |
| | 9G6 | 2.9 | + | − | − | − | N/A |
| | 6D9 | 0.2 | − | − | − | − | N/A |
| | 15C10 | 0.2 | − | − | − | − | N/A |
| | 14F5 | 0.2 | − | − | − | − | N/A |
| | 5H1 | 1.4 | ++ | +++ | ++ | +++ | + |
| | 17G1 | 0.2 | − | − | − | − | N/A |
| P2 | 11G1 | 4.8 | + | − | + | − | + |
| P3 | 9C1 | 1.0 | + | +++ | + | +++ | + |
| | 5D2 | 11.9 | + | − | − | − | ND |
| | 2H2 | 0.1 | + | − | − | − | N/A |
| | 12D4 | 5.3 | + | +++ | + | +++ | + |
| | 4E1 | 0.5 | − | +++ | − | − | N/A |
| | 10E2 | 0.1 | − | − | − | − | N/A |
| | 4E9 | 57.5 | +++ | − | + | − | + |
| | 12C2 | 0.3 | + | +++ | + | +++ | N/A |
| P4 | 16A4 | 0.1 | − | − | − | − | N/A |
| | 63C6 | 0.3 | − | − | − | − | N/A |
| | 30A10 | 2.5 | + | − | + | − | + |
| | 10E7 | 0.3 | − | − | + | − | N/A |
| | 47D1 | 0.1 | − | − | − | − | N/A |
| | 54C5 | 0.1 | − | − | − | − | N/A |
| | 20B12 | 0.1 | − | − | − | − | N/A |
| | 38C2 | 0.2 | − | − | − | − | N/A |
| | 40F1 | 0.1 | − | − | − | − | N/A |
| | 58E8 | 0.2 | − | − | − | − | N/A |
| | 38D12 | 0.1 | − | − | − | − | N/A |
| | 5A8 | 0.1 | − | − | + | − | N/A |
| P5 | 13A11 | 0.1 | − | − | − | − | N/A |
| | 5C1 | 0.4 | − | − | − | − | N/A |
| | 16B5 | 0.4 | − | − | − | − | N/A |
| | 11D5 | 0.1 | − | − | − | − | N/A |
| | 10D5 | 31.0 | ++ | − | − | − | + |
| P6 | 18A3 | 0.1 | − | − | − | − | N/A |
| P7 | 1G1 | 0.2 | − | − | − | − | N/A |
| | 2C7 | 0.3 | − | − | − | − | N/A |
| | 4E3 | 0.6 | − | − | − | − | N/A |
| | 2G4 | 0.3 | − | − | − | − | N/A |
| | 3A7 | 0.3 | − | − | − | − | N/A |
| | 4H3 | 0.2 | − | − | − | − | N/A |

SUPPLEMENTARY TABLE 3
Filaria-reactive human IgE mAb target proteins
Table S3. Antigenic proteins identified by human IgE mAbs. ND: not determined due to low
expression of target proteins. The most prominent antigen(s) are listed for TTR-specific
IgE mAbs (such as 5H1, 9C1 and 18H7) that cross-react with multiple members of the TTR family.

| | | ImmunoCAP | Binding to filarial | | | | | |
|---|---|---|---|---|---|---|---|---|
| IgE | Subject | reactivity | worm extract | | EC50 | Mass | Antigen | |
| mAb | code | (kUA/L) | ELISA | Immunoblot | (ng/mL) | spectrometry | name | Sequence ID |
| 12D4 | P3 | 5.3 | + | +++ | 24.6 | WbSXP-1 | Antigen | AF063940.1 |
| | | | | | | | Wb14 | |
| 4E1 | P3 | 0.5 | − | +++ | 144.6 | WbSXP-1 | Antigen | AF063940.1 |
| | | | | | | | Wb14 | |
| 9C1 | P3 | 1 | + | +++ | 95.9 | TTR-76 | ? | VDM14676.1 |
| 12C2 | P3 | 0.3 | + | +++ | 95.9 | TTR-76 | ? | VDM14676.1 |
| 5H1 | P1 | 1.4 | ++ | +++ | 6.5 | TTR-79 | ? | EJW78979.1 |
| 18H7 | P1 | 0 | + | +++ | 43.3 | TTR-79, | ? | EJW86262.1, |
| | | | | | | TTR-62 | | EJW78979.1 |
| 1A5 | P1 | 14.2 | ++ | +++ | ND | TTR-77 | ? | VDM13477.1 |
| 10D5 | P5 | 31 | ++ | − | ND | TTR-32 | ? | VDM07632.1 |
| 11G1 | P2 | 4.8 | + | − | ND | TTR-90 | ? | VDM14190.1 |
| 10H9 | P1 | 28.6 | ++ | − | ND | TTR-04 | ? | EJW80404.1 |
| 14B2 | P1 | 23.9 | + | +++ | ND | TTR-61 | ? | EJW84161.1 |
| 30A10 | P4 | 2.5 | + | − | ND | TTR-16 | ? | EJW86116.1 |
| 18D4 | P1 | 3.5 | + | − | ND | TTR-08 | ? | VDM13708.1 |
| 2E6 | P1 | 465.7 | +++ | +++ | 7.7 | Ladder | Nematode | AAG31482.1 |
| | | | | | | protein, | polyprotein | |
| | | | | | | gp15/400 | allergen-1 | |
| | | | | | | | (NPA-1) | |
| 4E9 | P3 | 57.5 | +++ | − | 26.7 | Macrophage | MIF-1 | EJW88743.1 |
| | | | | | | Migration | | |
| | | | | | | Inhibitory | | |
| | | | | | | Factor | | |
| | | | | | | (MIF) | | |
| 11H12 | P1 | 12 | ++ | +++ | 1.7 | 24 kDa | Alt-1, P22 | VIO90327.1 |
| | | | | | | secreted | | |
| | | | | | | protein | | |

TABLE S4
Supplementary Table 4. Sequence features of filaria-reactive human IgE mAbs

| | | Heavy | V gene | | J gene | | | SEQ | | | | | | | SEQ | |
|---|---|---|---|---|---|---|---|---|---|---|---|---|---|---|---|---|
| IgE | | chain | % iden- | | % iden- | | | ID | HCDR | Light chain | V gene % | | J gene % | | ID | LCDR |
| Mab | Light | V gene | tity | J gene | tity | D gene | Junction | NO: | length | V gene | identity | J gene | identity | Junction | NO: | length |
| 4E1 | κ | V1-46*01 F, | 96.18 | J4*02 F | 93.75 | D3-22*01 | CATLYYYD | 1 | 8.8.15 | V3-20*01 F | 92.2 | J1*01 F | 100 | CQQYGNSP | 17 | 7.3.9 |
| | | or V1- | | | | F | DSGYYTDY | | | | | | | WTF | | |
| | | 46*03 F | | | | | W | | | | | | | | | |
| 12D4 | λ | V1-45*02 F | 97.57 | J4*02 F | 93.75 | D3-22*01 | CAVAYHYD | 2 | 8.8.15 | V1-40*01 F | 96.18 | J2*01 F, | 94.74 | CQSYDSSL | 18 | 9.3.12 |
| | | | | | | F | SSGPYFDH | | | | | or | | SGPVVF | | |
| | | | | | | | W | | | | | J3*01 F | | | | |
| 5H1 | κ | V3-30*03 F, | 90.28 | J4*02 F | 85.11 | D3-16*01 | CAKDGPYA | 3 | 8.8.16 | V3-15*01 F | 92.47 | J4*01 F | 100 | CQQYHSWP | 19 | 6.3.10 |
| | | or V3- | | | | F | YRWGGFLD | | | | | | | PLTF | | |
| | | 30*18 F or | | | | | DW | | | | | | | | | |
| | | V3-30- | | | | | | | | | | | | | | |
| | | 5*01 F | | | | | | | | | | | | | | |
| 9C1 | κ | V3-23*01 F, | 92.01 | J6*02 F | 79.03 | D6-19*01 | CAKQKREC | 4 | 8.8.18 | V2-28*01 F, | 97.62 | J2*01 F | 97.3 | CMQALQTP | 20 | 11.3.9 |
| | | or V3- | | | | F | SGWCKGWG | | | V2D-28*01 F | | | | YTF | | |
| | | 23D*01 F | | | | | LDVW | | | | | | | | | |
| 14C10 | λ | V3-21*01 F | 93.06 | J3*02 F | 96 | D4-17*01 | CARDTYGD | 5 | 8.8.14 | V3-25*03 F | 89.25 | J3*02 F | 89.47 | CHVVDRTE | 21 | 6.3.12 |
| | | | | | | F | TDDAFDIW | | | | | | | TYRSVF | | |
| 7G12 | κ | V1-18*01 F | 89.24 | J4*02 F | 82.98 | D5-12*01 | CARGRDSP | 6 | 8.8.9 | V1-5*03 F | 92.83 | J2*01 F | 86.84 | CQQYDHFP | 22 | 6.3.9 |
| | | | | | | F | DWH | | | | | | | HTF | | |
| 1A5 | λ | V3-30*03 | 96.88 | J4*02 F | 56 | D3-22*01 | CAKAMDDS | 7 | 8.8.15 | V3-19*01 F | 97.13 | J1*01 F | 100 | CNSRDSSG | 23 | 6.3.12 |
| | | F, or V3- | | | | F | SGYYCPDY | | | | | | | NHLYVF | | |
| | | 30*18 F or | | | | | W | | | | | | | | | |
| | | V3-30- | | | | | | | | | | | | | | |
| | | 5*01 F | | | | | | | | | | | | | | |
| 10H9 | λ | V3-11*06 F | 89.93 | J1*01 F | 66.67 | D2-8*02 | CAKDYCGS | 8 | 8.8.20 | V3-21*01 F | 93.91 | J1*01 F | 94.74 | CQVWDNTN | 24 | 6.3.13 |
| | | | | | | F | GACYTADP | | | | | | | DHPSYVF | | |
| | | | | | | | GFFHQW | | | | | | | | | |
| 11G1 | λ | V5-51*03 F | 88.89 | J6*02 F | 58.14 | ND | CAREVYVA | 9 | 8.8.18 | V2-23*01 F, | 88.19 | J1*01 F | 86.11 | CCSYAGGN | 25 | 9.3.10 |
| | | | | or | | | STDSDYYG | | | OR | | | | TYVF | | |
| | | | | J6*04F | | | MDV | | | V2-23*02 F | | | | | | |
| | | | | | | | | | | or | | | | | | |
| | | | | | | | | | | V2-23*03 F | | | | | | |
| 14B2 | κ | V3-53*02 F | 87.37 | J4*02 F | 74.07 | D5-24*01 | CVRHGDGW | 10 | 8.7.12 | V3-15*01 F | 94.62 | J4*01 F | 71.05 | CQQYNNWP | 26 | 6.3.9 |
| | | | | | | | NYVDSW | | | | | | | LTF | | |
| 10D5 | κ | V3-21*01 F | 90.97 | J6*03 F | 83.87 | D3-22*01 | CARVMVVG | 11 | 8.8.15 | V3-20*01 F | 94.68 | J1*01 F | 97.37 | CQLYGTSP | 27 | 7.3.10 |
| | | | | | | F | LYYYYMDI | | | | | | | TWTF | | |
| | | | | | | | W | | | | | | | | | |
| 18D4 | κ | V4-34*02 | 91.23 | J4*02 F | 77.08 | D1-1*01 | CGRGRGYT | 12 | 8.7.10 | V1-39*01 F, | 93.55 | J2*01 F, | 88.57 | CQQSYSGP | 28 | 6.3.9 |
| | | F, or V4- | | | | F | WNDW | | | or | | or | | PTF | | |
| | | 34*08 F | | | | | | | | V1D-39*01 F | | J2*02 (F) | | | | |
| 30A10 | κ | V3-21*01 F | 97.92 | J6*03 F | 87.1 | D6-13*01 | CARDGPPI | 13 | 8.8.20 | V1-9*01 F | 98.57 | J2*02 (F) | 97.22 | CQHLNSYR | 29 | 6.3.8 |
| | | | | | | F | VAAGLIYY | | | | | | | TF | | |
| | | | | | | | YYMGVW | | | | | | | | | |
| 4E9 | λ | V4-34*02 F | 92.28 | J6*02 F | 93.55 | D6-13*01 | CASSGESS | 14 | 8.7.19 | V1-47*01 F, | 92.6 | J3*02 F | 89.47 | CAAWDDIL | 30 | 8.3.12 |
| | | | | | | F | RRGYFYYY | | | V1-47*02 F | | | | SGPRVF | | |
| | | | | | | | GMDVW | | | | | | | | | |
| 2E6 | λ | V1-69*01 F | 91.32 | J4*02 F | 100 | D5-18*01 | CASYPYGK | 15 | 8.8.12 | V1-51*01 F | 94.39 | J3*02 F | 97.14 | CGAWDLGL | 31 | 8.3.11 |
| | | | | | | F | YYFDYW | | | | | | | NAGVF | | |
| 11H12 | λ | V3-11*01 F | 89.24 | J4*02 F | 85.11 | ND | CARDWGTT | 16 | 8.8.13 | V4-60*03 F | 86.74 | J3*02 F | 75.68 | CFTWDSDS | 32 | 7.7.9 |
| | | | | | | | LVTFDLW | | | | | | | RVF | | |

Human IgE variable gene sequence characteristics. Antibody germline gene segment usages and their percent identity are shown for variable (V), diverse (D), and joining (J)
regions of both light and heavy chains based on the ImMunoGeneTics (IMGT) database. The number of amino acids that make up the CDRs (HCDR & LCDR length) and the amino
acids making up the junction is shown. (SEQ ID NOS: 1-32)

TABLE 1
NUCLEOTIDE SEQUENCES FOR ANTIBODY VARIABLE REGIONS

| | SEQ ID | | |
|---|---|---|---|
| Clone | NO: | Chain | Variable Sequence Region |
| 2E6 | 33 | heavy | CAGGTGCAGCTGGTGCAGTCTGGGGCTGAGGTGAAGAAGCCTGGGTCCTCGCTGAAGGTCT |
| | | | CCTGCAAGGCTTCTGGAGGCATCCTCAGTAGTCATGTTATCAGCTGGGTGCGACAGGCCCCG |
| | | | GGACAAGGCCTTGAGTGGATGGGAGGGGTCATCCCTGTCTTTGGTTCAACAACCTACGCACC |
| | | | GAAGTTCCAGGACAGACTCACAGTTACCGCGGACGAATCCACGAATACAGCCTACATGGACC |
| | | | TGAGCAGCCTGAGCTCTGAGGACACGGCCATTTATTACTGTGCGTCTTACCCCTATGGCAAA |
| | | | TACTACTTTGACTACTGGGGCCAGGGAACCCTGGTCACCGTCTCCTCAG |
| | 34 | light | CAGTCTGTGTTGACTCAGCCGCCCTCAGTGTCTGCGGCCCCAGGACAGAAGGTCACCATCTC |
| | | | CTGCTCTGGAAACAGCTCCAACATTGGCAGTAATTATGTATCCTGGTACCAGCAGGTCCCAGG |
| | | | AACAGCCCCCAAACTCCTCATTTATGACAATTTTAAGCGACCCTCAGACATTCCTGACCGATT |
| | | | CTCTGGCTCCAAGTCTGGCACGTCAGCCACCCTGGGCATCACCGGACTCCAGCCTGGGGACG |
| | | | AGGCCGATTATTACTGCGGAGCATGGGATCTCGGCCTCAACGCTGGGGTGTTCGGCGGAGG |
| | | | GACCAAACTGACCGTCCTAG |
| 4E9 | 35 | heavy | CAGGTGCAGCTACAACAGTGGGGCGCAGGCCTGTTGAAGCCTTCGGAGACCCTGTCCCTCAC |
| | | | GTGCGCTGTCTATGGTGGGTCCCCAAGGGGATACTACTGGAGCTGGATCCGCCAGCCCCCA |
| | | | GGGAAGGGACTGGAATGGATTGGGGAAATCAATCACAGTGGAACCACCAACTACAACCCGTC |
| | | | CCTCACGAGTCGAGTCACCATATCAGCAGACACGTCCAAGAGCCAATTTTCCCTGAGGCTGAC |
| | | | GTCTGTGACCGCCGCGGACACGGCTATATATTTCTGTGCGAGCAGCGGGGAAAGCAGCAGAC |
| | | | GGGGCTACTTCTACTATTACGGTATGGACGTCTGGGGCCAAGGGACCACGGTCACCGTCTCC |
| | | | TCA |
| | 36 | light | CAGTCTGTGCTGACTCAGCCACCCTCAGCGTCTGGGACCCCCGGGCAGACGGTCACCATCTCT |
| | | | TGTTCTGGCATCAGCTCCAACAACGAAAATTATTATGTTTATTGGTACCAGCAGCTCCCAGGA |
| | | | GCGGCCCCCAGGCTCCTCATCTATAGAAACAGTCAGCGGCCCTCAGGGGTCCCTGACCGATT |
| | | | CTCAGGCTCCAAGTCTGACACCTCAGCCTCCCTGGCCATCAGCGGGCTCCGGTCCGAGGATG |
| | | | AGGCTGATTATTACTGTGCAGCGTGGGATGACATCCTGAGTGGTCCGAGGGTGTTCGGCGGA |
| | | | GGGACCAAGTTGACCGTCCTAC |
| 4G8 | 37 | heavy | CAGGTGCAGTTGGTGCAGTCTGGGGCTGAGGTGAAGAAGCCTGGGTCCTCAGTGAAGGTCT |
| | | | CCTGCAAGGCTTCTGAAGGCACCTTCAGCAGCTGTGCTATCAGCTGGGTGCGACAGGCCCCT |
| | | | GGACAAGGGCTTGAGTGGATGGGAGGGATCATCCCTATGTTTGCTACAACACGCTACGCCCA |
| | | | GAAGTTCCAGGACAGAGTCACCATTACCACGGACGAATCGACGAACACAGCCTACATGGAGC |
| | | | TGAGCAGCCTGAGATCTGAGGACACGGCCATTTATTACTGTGCGAGAGACGGGGGGGGGAG |
| | | | CTTTTGTGGTGGTGATTGTTATTTCGACTTATGGGGCCAGGGAACCCTGGTCACCGTCTCCTC |
| | | | AG |
| | 38 | light | GAAATAGTGATGACGCAGTCTCCAGCCACCCTGTCTGTGTCTCCAGGGGAAAGAGCCACCCT |
| | | | CTCCTGCAGGGCCAGTCAGAGTGTTAACAGCAACTTGGCCTGGTATCAGCAGAAACCTGGCC |
| | | | AGGCTCCCAGGCTCCTCATCTTTGGTGCATCTATCAGGGCCACTGGTATCCCAGCCAGGTTCA |
| | | | GTGGCAGTGGGTCTGGGACGGAGTTCACTCTCACCATCAGCAGCCTGCAATCTGAAGATTTT |
| | | | GCAGTGTATTACTGTCAGCAGTATAATAACTGGCCTCCCACTTTCGGCCCTGGGACCAAAGTG |
| | | | GATATCAAAC |
| 7G12 | 39 | heavy | CAGGTTCAGCTGGTGCAGTCTGGAGTTGACGTGAAGAAGCCTGGGGCCTCAGTGAAACTCTC |
| | | | CTGCAAGACTTCTGGTTACACCTTTACTAATTATGGTATTACTTGGGTGCGACAGGCCCCTGG |
| | | | ACAAGGGCTTGAGTGGATGGGCTGGATCAGCACTTACGATGGTGCCACAAACTATAGCCAGA |
| | | | ATCTCCAGGGCAGAATCATCATGACCACTGACACATCCAAGAGGACAGCCTATCTGCAGATGA |
| | | | GGAGTCTGAGATCTGACGACACGGCCGTCTATTACTGTGCGAGGGGACGAGATAGTCCGGAC |
| | | | CACTGGGGCCAGGGAACCCTGGTCACCGTCTCCTCA |
| | 40 | light | GACATCCAGATGACCCAGTCTCCTTCCACCCTGTCTGCATCTGTAGGAGACAGAGTCACCATC |
| | | | ACTTGCCGGGCCAGTCAGAGTATTAATAGGTGGTTGGCCTGGTATCAGCAGAAACCAGGGAC |
| | | | AGCCCCTAAACTCCTCATCTTTAAGGCGTCTACTCTAGACAGTGGTGTCCCAGCGAGGTTCAG |
| | | | CGGCACTGGATCTGAGACAGAATTCTCTCTGACCATCAACAGCCTGCAGCCTGATGATTTTGC |
| | | | AACTTATTACTGCCAACAGTATGATCATTTTCCGCACACTTTTGGCCCGGGGACCAAACTGGA |
| | | | CATCAAA |
| 11H12 | 41 | heavy | CAGGTCCAACTGGTGGAGTCTGGGGGAGACTTGGTCACGCCTGGAGGGTCCCTGAGACTGT |
| | | | CGTGTGCAGCCTCGGGATTCGCCTTTAGCGGCTACTACATGAGTTGGATCCGCCAGGCTCCA |
| | | | GGGAAGGGGCTGGAATGGATCTCATACATTAATAGTAACGGTCTTACCATCTCCTACGCGGAC |
| | | | TCTGTGAAGGGCCGATTCACCGTCTCCAGGGACAATGCCAAGAACTCACTGTTTCTGCAAATG |
| | | | AGCTCCCTGAGAGCCGAGGACACGGCCATATATTACTGTGCGCGAGATTGGGGGACAACATT |
| | | | GGTAACTTTTGACCTCTGGGGCCAGGGAACCCTGGTCACCGTCTCCTCA |
| | 42 | light | CAGCCTGTGCTGACTCAATCATCCTCTGCCTCTGCTTCCATGGGATCCTCGGCCAAGCTCACC |
| | | | TGTACTCTGAGTAGTGGCCACAGAGGCTACAACATCGCTTGGCTTCAGCAGCATCCAGGGAA |
| | | | GGCCCCTCTCTATTTGACGAATCTTGAGGGTAGTGACTCCTACAAGAACGATCGCCTCACAGT |
| | | | CTCCAGCTCTGGGGCTGACCGCTACCTCACCATCTCCAACCTCCAGCCTGAAGATGAGGCTA |
| | | | CCTATTACTGCTTCACCTGGGACAGCGACTCCCGCGTCTTCGGGGGGGGACGCACCTGACC |
| | | | GTCCTG |
| 12F9 | 43 | heavy | CAGGTGCAGCTGGTGGAGTCTGGGGGAGACTTGGTCAAGCCTGGAGGGTCCCTGAGAATCT |
| | | | CCTGTGCAGCCTCTGGATTCAGCTTCAGTGACTACTACATGAGTTGGATCCGCCAGGCTCCAG |
| | | | GGAAGGGGCTTGAATGGGTTGCGTATATTAGTGGATCCAGTGCCTACACAAGCTACGCGGAC |
| | | | TCTGTGAAGGGCCGCTTCTCCATCTCCAGAGACAACGCCAACAACTCACTCTTTCTACAAATG |
| | | | AACAGCCTGAGAGCCGAGGACACGGCTACATATTTCTGTGCGAAAGATTACTGTGGCAGTGG |
| | | | CGCCTGCTACACTGCGGACCCTGGCTTCTTCCATCAATGGGCCAGG |
| | 44 | light | TCCTATGTTCTGACTCAGCCGCCCTCAGTGTCGGTGGCCCCAGGAAAGACGGCCACGATTTC |
| | | | CTGTGGGGGAAACAACATTGGAAGTAAAAGTGTGCACTGGTATCAGCAGAAGCCAGGCCAGG |
| | | | CCCCTATAGTGGTCATCTATTATGATAGCGACCGGCCCTCAGGGATCCCTGAGCGATTTTCTG |
| | | | GAATCAATTCTGGGAACACGGCCACCCTGACCATCAGCAGGGTCGAAGCCGGGGATGAGGCC |
| | | | GACTATTATTGTCAGGTGTGGGACAATACTAATGATCATCCCTCTTATGTCTTCGGAGCTGG |
| | | | GACCAAGGTCACCNTCCTAG |
| 14C10 | 45 | heavy | GAGGTGCAGCTGGTGCAGTCGGGGGGAGACCTGGTCAAGCCTGGGGGGTCCCTGAGACTCT |
| | | | CCTGTGCAGCCTCTGGATTCAGTTTCAGTAATTATCGAATGAACTGGGTCCGCCAGGCTCCAG |
| | | | GGAAGGGGCTGGAGTGGGTCTCATCCATTACTGGCAGTAGTAGTTACATCTACTACGCAGACT |
| | | | CCGTGCAGGGCCGATTCACCATCTCCAGAGACAACGCCAAGAACTCAATGTCTCTGCAAATGA |
| | | | AGAGCCTGAGAGTCGAGGACACGGCTGTGTATTACTGTGCGAGAGATACGTACGGTGACACC |
| | | | GATGATGCTTTTGATATTTGGGGCCAAGGGACAATGGTCACCGTCTCCTCAG |
| | 46 | light | TCCCTTGAGTTGATACAGCCACCCTCGGTGGCGGTGTCCCCAGGACAGACGGCCAGGATCAG |
| | | | CTGCTCTGGAAATGCATTGGCAAAGGAATATGCTAATTGGTACCAGCAGAGGCCAGGCCAGG |
| | | | CCCCTGTTTTGGTGATTTTTAAAGACATTGAGAGGCCCACCGGGATTCCTGAGCGATTCTCTG |
| | | | GCTCCAGCTCAGGGACAACACTCACGTTGACCATCAGTGGAGTCCAGGCAGAAGACGAGGCT |
| | | | AATTATTACTGTCACGTAGTTGACAGGACTGAGACGTATCGAAGTGTCTTCGGCGGAGGGACC |
| | | | AAGCTGACCGTCCTAG |
| 1A5 | 47 | heavy | GAAGTGCAGCTGGTGGAGTCTGGGGGAGGCGTGGTCCAGCCTGGGAGGTCCCTGAGACTCT |
| | | | CCTGTGCAGGCTCTGGATTCACCTTCAGTACCTATGTCATGCACTGGGTCCGCCAGGCTCCA |
| | | | GGCAAGGGGCTGGAGTGGGTGGCAGTTATATCATATGATGGAACTAATAAATACTATGCAGAC |
| | | | TCCATGAAGGGCCGATTCACCATCTCCAGAGACAATTCCAAGAACACGCTGTATCTGCAACTG |
| | | | AACCGCCTGAGAGCTGAGGACACGGCTGTGTATTACTGTGCGAAAGCAATGGATGATAGTAG |
| | | | TGGTTATTATTGTCCTGATTACTGGGCCANGNA |
| | 48 | light | TCTTCTGAGCTGACTCAGGACCCTGCTGTGTCTGTGGCCTTGGGACAGACAGTCAAGATCACA |
| | | | TGCCAAGGAGACAGCCTCAGAAACTATTTTGCAAACTGGTACCAGCAGAAGCCAGGACAGGC |
| | | | CCCTGTTCTTGTCATCTATGGTCAAAACAACCGGCCCTCAGGGATCCCAGACCGATTCTCTGG |
| | | | CTCCACCTCAGGAAACACAGGTTCCTTGACCATCACTGGGGCTCAGGCGGAAGATGAGGCTG |
| | | | ACTATTACTGTAACTCCCGGGACAGCAGTGGTAACCATCTTTATGTCTTCGGAACTGGGACCA |
| | | | AGGTCACCGTCCTAG |
| 5H1 | 49 | heavy | CAGGTGCAGCTGGTGCAGTCGGGGGGGGGCGTGGTCCAGCCTGGGAGGTCATTGAGACTCT |
| | | | CATGTAATGCCTCTGGATTCACCTTCAGTAAATATGGCATGCACTGGGTCCGCCAGGCTCCAG |
| | | | GCAAGGGGCTCGAGTGGGTGGCAGTTATCACACATGATGGAGGTGATAAACACCATGCAGAC |
| | | | TCCGTGAAGGGCCGATTTAGCATCTCCAGAGACAATTCCAAGGACATCCTGTATCTGCAAATG |
| | | | GACAGCCTGAGAACTGAGGACACGGCTATTTATTACTGTGCGAAAGACGGCCCCTATGCTTAC |
| | | | CGTTGGGGGGGTTTCTTGGACGACTGGGGCCAGGGAACTCCGGTCACCGTCTCCTCAG |
| | 50 | light | GAAATAGTGATGACGCAGTCTCCAGTCACCCTGTCTGTCTCTCCAGGGGAGAGAGTCACCCT |
| | | | CTCCTGCAGGGCCAGTGAGAGTATTGGCAGCAGTTTAGCCTGGTACCAACAAAAACCTGGCC |
| | | | AGGCTCCCAGGCTCCTCATCTATGGAGCCTCCACCAGGACCACTGGTATCCCAGCCAGATTC |
| | | | AGTGGCAGTGGGTCTGGGACAGAGTTCACTCTCGCCATCAGCAGCCTGCAGTCTGAAGATTA |
| | | | TGCAGTTTATTTCTGTCAGCAGTATCATAGCTGGCCCCCGCTCACTTTCGGCGGAGGGACCAA |
| | | | GGTGGAGATCAAAC |
| 9C1 | 51 | heavy | GAGGTGCAGCTGTCGGAGTCTGGGGGAGGCTTGGTACAGCCGGGGGGGTCCCTGAGACTCT |
| | | | CCTGTGCAGCCTCTGGGTTCACCTTTAGCGACTTTGCCATGAGTTGGGTCCGCCAGGCTCCA |
| | | | GGGAAGGGACTGGAGTGGGTCTCAGCTATCGGAGCTGGTGGTTCTAACACATACAACGCAGA |
| | | | CTCCGTGAAGGGCCGGTTCACCATCTCCAGGGACAATTCCGCGAACACGCTGTATTTGCAAAT |
| | | | GAATAGCCTGAGAGCCGAGGACACGGCCCTATATTACTGTGCGAAACAGAAACGAGAATGCA |
| | | | GTGGCTGGTGCAAAGGATGGGGTTTGGACGTCTGGGGCCAAGGGACCACGGTCACCGTCTC |
| | | | CTCA |
| | 52 | light | GATATTGTGATGACTCAGTCTCCACTCTCCCTGCCCGTCACCCCTGGAGAGCCGGCCTCCAT |
| | | | CTCCTGCAAGTCTAGTCAGAGCCTCCTGGATAGTAAGGGATCCACCTATTTGGATTGGTACCT |
| | | | GCAGAAGCCAGGGCAGTCTCCACAGCTCCTGATCTATTTGGCTTCTTATCGGGCCTCCGGGG |
| | | | TCCCTGACAGGTTCAGTGGCAGTGGATCAGGCACAGATTTTACACTGAAAATCAGCAGAGTGG |
| | | | AGGCTGAGGATGTTGGGGTTTATTACTGCATGCAAGCTCTACAAACTCCTTACACTTTTGGCC |
| | | | AGGGGACCAGGCTGGAGATCAAAC |
| 10D5 | 53 | heavy | GAGGTGCAGCTGGTGGAGTCGGGGGGAGGCCTGGTCAAGCCTGGGGGGTCCCTGAGACTC |
| | | | TCCTGTGCAGCCTCTGGATTCACCTTCAGTTCGTCTATGATGACCTGGGTCCGCCAGGCTCCA |
| | | | GGGAAGGGGCTGGAGTGGGTCTCATCTATTGGAACTGGTAGTAACTACAAACACTACGCAGA |
| | | | CTCACTGAGGGGCCGATTCACCGTCTCCCGAGACAACGCCAAGAACTCACTGTTTCTACAAAT |
| | | | GAACAGCCTGAGACCCGAGGACACGGCTGTCTATTACTGTGCGAGAGTAATGGTAGTAGGGC |
| | | | TTTACTACTACTACATGGACATCTGGGGCACAGGGACCACGGTCACCGTCTCCTCA |
| | 54 | light | GAAATTGTGTTGACGCAGTCTCCAGGCACCCTGTCTTTGTCTCCAGGGGAAAGAGCCGCCCT |
| | | | CTCCTGCAGGGCCAGTCAGAATGTTAGTAGCAACTTCTTAGCCTGGTACCAGCAGAACCCTG |
| | | | GCCAGGCTCCCAGGCTCCTCTTCTATGGTGCGTCCATGAGGGCCACTGGCATCCCAGACAGG |
| | | | TTCAGTGGCAGTGGGTCTGGGACAGACTTCACTCTCACCATCAGTAGACTGGAGCCTGAAGA |
| | | | TTTTGCAGTGTATTACTGTCAACTGTACGGTACCTCACCAACGTGGACGTTCGGCCAAGGGAC |
| | | | CAAGGTGGAGATCAAAC |
| 11G1 | 55 | heavy | GAGGTGCAGCTGGTCCAGTCTGGACCAGTGCTAAAAAAGCCGGGGGAGTCTCTGAAGATCTC |
| | | | CTGTAGGGGCTCTGGATACAGGTTCAACACTTATTGGGTCGCCTGGGTGCGCCAGATGCCCG |
| | | | GGAAAGGTCTGGAGTGGATGGGAATGATCTATCCGGGTGACTCGGATACGAAATATAGTCCG |
| | | | TCCTTCCAAGGCCAAGTCACCATCTCAGCCGACAAGTCCAGCAATACCGCCTACCTACAGTGG |
| | | | AGTAGTCTGAAGGCCTCGGACACCGCCATGTATTATTGTGCGAGAGAAGTATATGTGGCTTCG |
| | | | ACTGATAGTGACTATTACGGTATGGACGTCNNNGCCTAGGACCA |
| | 56 | light | AGTCTGCNCCTGACTCAACCTGCCTCGCGTGTCTGGGTCTCCTGGACAGTCGATCACCATCT |
| | | | CCTGCACTGGAACCAGCAATGATGTTGGGCGTTCTGACCTTGTCGCCTGGTACCAACAACAC |
| | | | CCCGACAAGGCCCCCAGACTCATTATTTATGAGTCCAGTAAGCGGCCCTCAGGGGTTTCTGC |
| | | | CCGCTTCTCTGGCTCCNGGTCTGGCATCACGGCCTCCCTGACAATCTCTGACCTCCAGGCTG |
| | | | AAGACGAGGCTGACTATTACTGCTGTTCATATGCAGGTGGTAACACTTATGTCTTCGGCACCG |
| | | | CGACCGGGGTCACCGTCCTAG |
| 14B2 | 57 | heavy | GAGGTGCAGCTGGTGGAGACTGGAGGAGGCTTGATTCAGCCGGGGGGGTCCCTGCGACTCT |
| | | | CCTGTGCAGCCTCTGGGTTCAGCGTCAGTACGAGGTTCATGAGCTGGGTCCGCCAGGCTCCA |
| | | | GGTCAGGGACTGGAGTGGGTCTCAGTCGTCTATAAAGATGGTGACACCTTCAACTCGGACTC |
| | | | CGTGAAGGGCCGATTCAGCATCTCCAGAGACAATTCCAAGAACACAGTGTTTCTTCAAATGAA |
| | | | CAGACTGAGAGTCGAAGACACTGCCGTATACTTCTGTGTGCGACATGGCGATGGTTGGAATTA |
| | | | CGTCGACTCCTGGGGCCTGGAAC |
| | 58 | light | GAAATAGTGATGACGCAGTCTCCAGCCACCCTGTCTGTGTCTCCAGGGGAAGGAGCCACCCT |
| | | | CTCCTGCAGGGCCAGTCACAGTCTTAGTAGTCACTTAGCCTGGTACCAGCAAAAACCTGGCCA |
| | | | GGCTCCCAGGCTCCTAATATATGATGCATCCGTCAGGGCCACTGATATCCCAGCCAGGTTCA |
| | | | GTGGCAGTGGGTCTGGGACAGAGTTCACTCTCACCATCAGCAGCCTGCAGTCTGAAGATTTT |
| | | | GCAGTTTACTACTGTCAGCAATATAATAACTGGCCGCTCACTTTCGGCGGAGGGACCAAGCNG |
| | | | AGATCAANCG |
| 12C2 | 59 | heavy | GAGGTGCAGCTGTCGGAGTCTGGGGGAGGCTTGGTACGGCCGGGGGGGTCCCTGAGACTCT |
| | | | CCTGTGCAGCCTCTGGGTTCACCTTTAGCGACTTTGCCATGAGTTGGGTCCGCCAGGCTCCA |
| | | | GGGAAGGGACTGGAGTGGGTCTCAGCTATCGGAGCTGGTGGTTCTAACACATACAACGCAGA |
| | | | CTCCGTGAAGGGCCGGTTCACCATCTCCAGGGACAATTCCGCGAACACGCTGTATTTGCAAAT |
| | | | GAATAGCCTGAGAGCCGAGGACACGGCCCTATATTACTGTGCGAAACAGAAACGAGAATGCA |
| | | | GTGGCTGGTGCAAAGGATGGGGTTTGGACGTCTGGGGCCAAGGGACCACGGTCACCGTCTC |
| | | | CTCA |
| | 60 | light | GATATTGTGATGACTCAGTCTCCACTCTCCCTGCCCGTCACCCCTGGAGAGCCGGCCTCCAT |
| | | | CTCCTGCAAGTCTAGTCAGAGCCTCCTGGATAGTAAGGGATCCACCTATTTGGATTGGTACCT |
| | | | GCAGAAGCCAGGGCAGTCTCCACAGCTCCTGATCTATTTGGCTTCTTATCGGGCCTCCGGGG |
| | | | TCCCTGACAGGTTCAGTGGCAGTGGATCAGGCACAGATTTTACACTGAAAATCAGCAGAGTGG |
| | | | AGGCTGAGGATGTTGGGGTTTATTACTGCATGCAAGCTCTACAAACTCCTTACACTTTTGGCC |
| | | | AGGGGACCAGGCTGGAGATCAAAC |
| 18D4 | 61 | heavy | CAGGTGCAGCTACAACAGTGGGGCGCAGGACTGTTGAAGCCATCGGAGACCCTGTCCCTCAC |
| | | | CTGCGCTGTCTATGGTGGGACGTTCAGTGATTACTTCTATACCTGGATCCGCCAGCTCCCAGG |
| | | | GAAGGGGCTGGAGTGGATTGGGGAAGTCAGTCATACTGGGAGGACCAGCTACATCCCGTCC |
| | | | CTCAAGAGTCGAGTCACCATGTCAGTAGACACGTCTAAAAACCAGTTCTCCCTGAACTTGAGC |
| | | | TCTGTGACCGACGCGGACACGGCTGTTTATTATTGTGGGAGAGGTCGGGGTTACACTTGGAA |
| | | | TGATTGGGGCCAGGGAACGCTAGTCACCGTCTCCTCAG |
| | 62 | light | GACATCCAGATGACCCAGTCTCCATCCTCCCTGTCTGCCTCTGTAGGAGACAGAGTCACCATC |
| | | | ACTTGTCGGGCAAGTCAGAGCATTAGAACCTCTTTAAATTGGTATCAACAAAGACCAGGGAGA |
| | | | GCCCCTAAACTCCTGATCTATGCTGCAACCAGTTTGCAAGGTGGGTTCCCGTCAAGGTTCAGT |
| | | | GGCAGTGGATCTGGGACAGATTTCACTCTCACCATCAGCAGTCTGCAACCCGAAGATTTTGCA |
| | | | ACTTACTACTGTCAGCAGAGCTACAGTGGCCCTCCAACTTTTGGCCAGGGGACCAAGTTGGAT |
| | | | TTCAAAC |
| 30A10 | 63 | heavy | GAGGTGCAGCTGGTGGAGTCTGGGGGAGGCCTGGTCAAGCCTGGGGGGTCCCTGAGACTCT |
| | | | CCTGTGCAGCCTCTGGATTCAGTTTCAGTAGCTATAGCATGAACTGGGTCCGCCAGGCTCCA |
| | | | GGGAAGGGGCTGGAGTGGGTCTCATCCATTAGTACTAGTGGTAATTACATATACTACGCAGAC |
| | | | TCAGTGAAGGGCCGATTCACCATCTCCAGAGACAACGCCAAGAACTCACTGTATCTGCAAATG |
| | | | AACAGCCTGAGAGCCGAGGACACGGCTGTATATTACTGTGCGAGAGATGGACCTCCGATAGT |
| | | | AGCAGCTGGCCTGATCTACTACTACTACATGGGCGTCTGGGGCAAAGGGACCACGGTCACCG |
| | | | TCTCCTCA |
| | 64 | light | GACATCCAGTTGACCCAGTCTCCATCCTCCCTGTCTGCATCTGTAGGAGACAGAGTCACCATC |
| | | | ACTTGCCGGGCCAGTCAGGGCATTAGCAGTTATTTAGCCTGGTATCAGCAAAAACCAGGGAAA |
| | | | GCCCCTAAGCTCCTGATCTATGCTGCATCCACTTTGCAAAGTGGGGTCCCATCAAGGTTCAGC |
| | | | GGCAGTGGATCTGGGACAGATTTCACTCTCACCATCAGCAGCCTGCAGCCTGAAGATTTTGCA |
| | | | ACTTATTACTGTCAACACCTTAATAGTTACCGGACTTTTGGCCAGGGGACCAAGCTGGAGATC |
| | | | AAAC |
| 18H7 | 65 | heavy | CAGGTGCAGCTTCAGGAGTCGGGCCCAGGACTGGTGAATCCTTCACAGGTCCTGTCC |
| | | | CTCACCTGCGCTGTCTCTGGTGACTCCATCGTGACTGGTAAGTACTACTGGAGCTGG |
| | | | ATCCGCCAACAACCAGGCAAGGGCTTGGAGTGGATTGGATTCATTGATCACAGTGGA |
| | | | ACTACCTTCTACAACCCGTCCCTCAAGAGTCGCCTCTCCATGTCAGTAGACACGTCTA |
| | | | AGAACCACTTCTCCCTCCATCTAAAGTCTGTGACCGCCGCGGACACGGCCGTCTATT |
| | | | ATTGTGCGAGAGACCAGAAGTATGAGGGAGATAATTGGTTCGACCCCTGGGGCCAGG |
| | | | GAACCCTGGTAATCGTCTCCTCAG |
| | 66 | light | CAGTCTGTGCTGACTCAGCCACCCTCAGCCTCTGGGACCCCCGGGCAGAGGGTCAC |
| | | | CATCTCTTGCTCTGGAAGCAGCTCCAACGTCGCATATAATAGTGTTAACTGGTACCAG |
| | | | CAAGTCCCAGGAACGGCCCCCAAACTCCTCCTCTACGGGACTAATCGGCGGCCCTCA |
| | | | GGGGTCCCTGACCGGTTTTCTGGCTCCAAGTCTGGCACCTCAGCCTCCCTGGCCATC |
| | | | AGTGGGCTCCAGTCTGAGGATGAGGCTGATTATTATTGTTCGGCGTGGGATGAGAGC |
| | | | CTGAGTGGTCCGGTCTTCGGCGGGGGGACCAAACTGACCGTCCTCG |
| 4E1 | 67 | heavy | CAGGTGCAGCTGGTGCAGTCTGGGGCTGAGGTGAAGAAGCCTGGGGCCTCAGTGAAGGTTT |
| | | | CCTGCAAGGCATCTGGATACACCTTCACCAGATACTATATGCACTGGGTGCGACAGGCCCCT |
| | | | GGACAAGGGCCTGAGTGGATGGGAATAATCAACCCTAGTGATGGTCGCACAGGCTACGCACA |
| | | | GAAGTTCCAGGGCAGAATCACGCTGACCAGGGACACGTCCACGAGCACAGTCTACATGGAGC |
| | | | TGAGCAGCCTGAAATCTGAGGACACGGCCATATATTACTGTGCGACCCTCTATTACTATGATG |
| | | | ATAGTGGTTATTACACTGACTACTGGGGCCAGGGAACCCTGGTCACCGTCTCCTCAG |
| | 68 | light | GAAATTGTGTTGACGCAGTCTCCAGGCACCCTGTCTTTGTCTCCAGGGGAAAGAGCCACCCT |
| | | | CTCCTGCAGGGCCAGTCAGAGTGTCAGCAACAACTACTTAGGCTGGTTCCAGCAGAAACCTG |
| | | | GCCAGGCTCCCAGGCTCCTCATCTATGGTGCATCAAGAAGGGCCACGGGCATCCCAGACAG |
| | | | GTTCACTGGTAGTGGGTCTGGGACAGACTTCACTCTCACCATCAGCAGACTGGAGCCTGAAG |
| | | | ATTTTGCAGTGTATTACTGTCAGCAGTATGGTAACTCACCGTGGACGTTCGGCCAAGGGACCA |
| | | | AGGTGGAAATCAAAC |
| 12D4 | 69 | heavy | CAGATGCAGCTGGTGCAGTCTGGGGCTGAGGTGAAGAAGACTGGGTCCTCAGTGAAGGTTTC |
| | | | CTGCAAGGCTTCCGGATACACCTTCACCTACCGCTACCTGCACTGGGTGCGACAGGCCCCCG |
| | | | GACAAGCGCTTGAGTGGATGGGTTGGATCACACCTTTCAATGATAACACCAACTACGCACAGA |
| | | | AATTCCAGGGCAGAGTCACCATTACCAGGGACAGGTCTATGAGCACAGCCTACATGGAGTTG |
| | | | AGCAGCCTGAGATCTGAGGACACAGCCATGTATTACTGTGCTGTGGCTTATCACTATGATAGT |
| | | | AGTGGTCCGTACTTTGACCACTGGGGCCAGGGAACCCTGGTCACCGTCTCCTCAA |
| | 70 | light | CAGTCTGTGCTGACGCAGCCGCCCTCAGTGTCTGGGGCCCCAGGGCAGAGGGTCATCATCT |
| | | | CCTGCACTGGGAGCAGCTCCAACATCGGGGCAGGTTATGATGTACACTGGTACCAGCAGCTT |
| | | | CCAGGAACAGCCCCCAAACTCCTCGTCTCTAGTAGCAGCCATCGGCCCTCAGGGGTCCCTGA |
| | | | CCGATTCTCTGCCTCCAAGTCTGGCACCTCTGCCTCCCTGGCCATCACTGGGCTCCAGGCTG |
| | | | AGGATGAGGCTGATTATTTCTGCCAGTCGTATGACAGCAGTCTGAGTGGTCCCGTGGTATTCG |
| | | | GCGGAGGGACCAAGCTGACCGTCCTAA |

TABLE 2
PROTEIN SEQUENCES FOR ANTIBODY VARIABLE REGIONS

| | SEQ ID | | |
|---|---|---|---|
| Clone | NO: | Chain | Variable Sequence |
| 2E6 | 71 | heavy | QVQLVQSGAEVKKPGSSLKVSCKASGGILSSHVISWVRQAPGQGLEWMGGVIPVE |
| | | | GSTTYAPKFQDRLTVTADESTNTAYMDLSSLSSEDTAIYYCASYPYGKYYEDYWG |
| | | | QGTLVTVSS |
| | 72 | light | QSVLIQPPSVSAAPGQKVTISCSGNSSNIGSNYVSWYQQVPGTAPKLLIYDNEKR |
| | | | PSDIFDRFSGSKSGTSATLGITGLQPGDEADYYCGAWDLGLNAGVEGGGTKLTVL |
| 4E9 | 73 | heavy | QVQLQQWGAGLLKPSETLSLTCAVYGGSPRGYYWSWIRQPPGKGLEWIGEINHSG |
| | | | TTNYNPSLTSRVTISADTSKSQFSLRLTSVTAADTAIYFCASSGESSRRGYFYYY |
| | | | GMDVWGQGTTVTVSS |
| | 74 | light | QSVLTQPPSASGTPGQTVTISCSGISSNNENYYVYWYQQLPGAAPRLLIYRNSQR |
| | | | PSGVPDRESGSKSDTSASLAISGLRSEDEADYYCAAWDDILSGPRVEGGGTKLTV |
| | | | L |
| 4G8 | 75 | heavy | QVQLVQSGAEVKKPGSSVKVSCKASEGTFSSCAISWVRQAPGQGLEWMGGIIPME |
| | | | ATTRYAQKFQDRVTITTDESTNTAYMELSSLRSEDTAIYYCARDGGGSFCGGDCY |
| | | | FDLWGQGTLVTVSS |
| | 76 | light | EIVMTQSPATLSVSPGERATLSCRASQSVNSNLAWYQQKPGQAPRLLIFGASIRA |
| | | | TGIPARFSGSGSGTEFTLTISSLQSEDFAVYYCQQYNNWPPTFGPGTKVDIK |
| 7G12 | 77 | heavy | QVQLVQSGVDVKKPGASVKLSCKTSGYTFTNYGITWVRQAPGQGLEWMGWISTYD |
| | | | GATNYSQNLQGRIIMTTDTSKRTAYLQMRSLRSDDTAVYYCARGRDSPDHWGQGT |
| | | | LVTVSS |
| | 78 | light | DIQMTQSPSTLSASVGDRVTITCRASQSINRWLAWYQQKPGTAPKLLIFKASTLD |
| | | | SGVPARFSGTGSETEFSLTINSLQPDDEATYYCQQYDHFPHTFGPGTKLDIK |
| 11H12 | 79 | heavy | QVQLVESGGDLVTPGGSLRLSCAASGFAFSGYYMSWIRQAPGKGLEWISYINSNG |
| | | | LTISYADSVKGRFTVSRDNAKNSLFLQMSSLRAEDTAIYYCARDWGTILVTFDLW |
| | | | GQGTLVTVSS |
| | 80 | light | QPVLTQSSSASASMGSSAKLTCTLSSGHRGYNIAWLQQHPGKAPLYLINLEGSDS |
| | | | YKNDRLTVSSSGADRYLTISNLQPEDEATYYCFTWDSDSRVFGGGTHLTVL |
| 12F9 | 81 | heavy | QVQLVESGGDLVKPGGSLRISCAASGFSFSDYYMSWIRQAPGKGLEWVAYISGSS |
| | | | AYTSYADSVKGRFSISRDNANNSLFLQMNSLRAEDTATYFCAKDYCGSGACYTAD |
| | | | PGFFHQWAR |
| | 82 | light | SYVLTQPPSVSVAPGKTATISCGGNNIGSKSVHWYQQKPGQAPIVVIYYDSQRPS |
| | | | GIPERFSGINSGNTATLTISRVEAGDEADYYCQVWDNENDHPSYVFGAGTKVTXL |
| 14C10 | 83 | heavy | EVQLVQSGGDLVKPGGSLRLSCAASGFSFSNYRMNWVRQAPGKGLEWVSSITGSS |
| | | | SYIYYADSVQGRFTISRDNAKNSMSLQMKSLRVEDTAVYYCARDTYGDTDDAFDI |
| | | | WGQGTMVTVSS |
| | 84 | light | SLELIQPPSVAVSPGQTARISCSGNALAKEYANWYQQRPGQAPVLVIFKDIERPT |
| | | | GIPERFSGSSSGTTLTLTISGVQAEDEANYYCHVVDRIETYRSVFGGGTKLTVL |
| 1A5 | 85 | heavy | EVQLVESGGGVVQPGRSLRLSCAGSGFTFSTYVMHWVRQAPGKGLEWVAVISYDG |
| | | | TNKYYADSMKGRFTISRDNSKNTLYLQLNRLRAEDTAVYYCAKAMDDSSGYYCPD |
| | | | YWAX |
| | 86 | light | SSELTQDPAVSVALGQTVKITCQGDSLRNYFANWYQQKPGQAPVLVIYGQNNRPS |
| | | | GIPDRESGSTSGNTGSLTITGAQAEDEADYYCNSRDSSGNHLYVFGTGTKVTVL |
| 5H1 | 87 | heavy | QVQLVQSGGGVVQPGRSLRLSCNASGFTFSKYGMHWVRQAPGKGLEWVAVITHDG |
| | | | GDKHHADSVKGRFSISRDNSKDILYLQMDSLRTEDTAIYYCAKDGPYAYRWGGEL |
| | | | DDWGQGTPVTVSS |
| | 88 | light | EIVMTQSPVTLSVSPGERVTLSCRASESIGSSLAWYQQKPGQAPRLLIYGASTRT |
| | | | TGIPARFSGSGSGTEFTLAISSLQSEDYAVYFCQQYHSWPPLTFGGGTKVEIK |
| 9C1 | 89 | heavy | EVQLSESGGGLVQPGGSLRLSCAASGFTFSDFAMSWVRQAPGKGLEWVSAIGAGG |
| | | | SNTYNADSVKGRFTISRDNSANTLYLQMNSLRAEDTALYYCAKQKRECSGWCKGW |
| | | | GLDVWGQGTTVTVSS |
| | 90 | light | DIVMTQSPLSLPVTPGEPASISCKSSQSILDSKGSTYLDWYLQKPGQSPQLLIYL |
| | | | ASYRASGVPDRESGSGSGTDETLKISRVEAEDVGVYYCMQALQTPYTFGQGTRLE |
| | | | IK |
| 10D5 | 91 | heavy | EVQLVESGGGLVKPGGSLRLSCAASGFTFSSSMMTWVRQAPGKGLEWVSSIGTGS |
| | | | NYKHYADSLRGRFTVSRDNAKNSLFLQMNSLRPEDTAVYYCARVMVVGLYYYYMD |
| | | | IWGTGTTVTVSS |
| | 92 | light | EIVLTQSPGTLSLSPGERAALSCRASQNVSSNFLAWYQQNPGQAPRLLFYGASMR |
| | | | ATGIPDRFSGSGSGTDETLTISRLEPEDFAVYYCQLYGISPTWTFGQGTKVEIK |
| 11G1 | 93 | heavy | EVQLVQSGPVLKKPGESLKISCRGSGYRENTYWVAWVRQMPGKGLEWMGMIYPGD |
| | | | SDTKYSPSFQGQVTISADKSSNTAYLQWSSLKASDTAMYYCAREVYVASTDSDYY |
| | | | GMDVAD |
| | 94 | light | SLXLTQPASVSGSPGQSITISCTGTSNDVGRSDLVAWYQQHPDKAPRLIIYESSK |
| | | | RPSGVSARFSGSXSGITASLTISDLQAEDEADYYCCSYAGGNTYVEGTATGVTVL |
| 14B2 | 95 | heavy | EVQLVETGGGLIQPGGSLRLSCAASGFSVSTRFMSWVRQAPGQGLEWVSVVYKDG |
| | | | DTFNSDSVKGRESISRDNSKNTVFLQMNRLRVEDTAVYFCVRHGDGWNYVDSWGL |
| | | | E |
| | 96 | light | EIVMTQSPATLSVSPGEGATLSCRASHSLSSHLAWYQQKPGQAPRLLIYDASVRA |
| | | | TDIPARFSGSGSGTEFTLTISSLQSEDFAVYYCQQYNNWPLTFGGGTKXRSX |
| 12C2 | 97 | heavy | EVQLSESGGGLVRPGGSLRLSCAASGFTFSDFAMSWVRQAPGKGLEWVSAIGAGG |
| | | | SNTYNADSVKGRFTISRDNSANTLYLQMNSLRAEDTALYYCAKQKRECSGWCKGW |
| | | | GLDVWGQGTTVTVSS |
| | 98 | light | DIVMTQSPLSLPVTPGEPASISCKSSQSILDSKGSTYLDWYLQKPGQSPQLLIYL |
| | | | ASYRASGVPDRFSGSGSGTDFTLKISRVEAEDVGVYYCMQALQTPYTFGQGTRLE |
| | | | IK |
| 18D4 | 99 | heavy | QVQLQQWGAGLLKPSETLSLTCAVYGGTFSDYFYTWIRQLPGKGLEWIGEVSHTG |
| | | | RTSYIPSLKSRVTMSVDTSKNQFSLNLSSVTDADTAVYYCGRGRGYTWNDWGQGT |
| | | | LVTVSS |
| | 100 | light | DIQMTQSPSSLSASVGDRVTITCRASQSIRTSLNWYQQRPGRAPKLLIYAATSLQ |
| | | | GGFPSRFSGSGSGTDETLTISSLQPEDFATYYCQQSYSGPPTFGQGTKLDEK |
| 30A10 | 101 | heavy | EVQLVESGGGLVKPGGSLRLSCAASGFSFSSYSMNWVRQAPGKGLEWVSSISTSG |
| | | | NYIYYADSVKGRFTISRDNAKNSLYLQMNSLRAEDTAVYYCARDGPPIVAAGLIY |
| | | | YYYMGVWGKGTTVTVSS |
| | 102 | light | DIQLTQSPSSLSASVGDRVTITCRASQGISSYAWYQQKPGKAPKLLIYAASTLQ |
| | | | SGVPSRFSGSGSGTDEILTISSLQPEDFATYYCQHLNSYRTFGQGTKLEIK |
| 18H7 | 103 | heavy | QVQLQESGPGLVNPSQVLSLTCAVSGDSIVIGKYYWSWIRQQPGKGLEWIGFIDH |
| | | | SGTTFYNPSLKSRLSMSVDTSKNHFSLHLKSVTAADTAVYYCARDQKYEGDNWED |
| | | | PWGQGTLVIVSS |
| | 104 | light | QSVLTQPPSASGTPGQRVTISCSGSSSNVAYNSVNWYQQVPGTAPKLLLYGINRR |
| | | | PSGVFDRFSGSKSGTSASLAISGLQSEDEADYYCSAWDESLSGPVFGGGTKLTVL |
| 4E1 | 105 | heavy | QVQLVQSGAEVKKPGASVKVSCKASGYTFTRYYMHWVRQAPGQGPEWMGIINPSD |
| | | | GRTGYAQKFQGRITLTRDISTSTVYMELSSLKSEDIAIYYCATLYYYDDSGYYTD |
| | | | YWGQGTLVTVSS |
| | 106 | light | EIVLIQSPGILSLSPGERATLSCRASQSVSNNYLGWFQQKPGQAPRLLIYGASRR |
| | | | ATGIPDRFTGSGSGTDETLTISRLEPEDFAVYYCQQYGNSPWTFGQGTKVEIK |
| 12D4 | 107 | heavy | QMQLVQSGAEVKKTGSSVKVSCKASGYTFTYRYLHWVRQAPGQALEWMGWITPEN |
| | | | DNTNYAQKFQGRVTITRDRSMSTAYMELSSLRSEDTAMYYCAVAYHYDSSGPYED |
| | | | HWGQGTLVTVSS |
| | 108 | light | QSVLTQPPSVSGAPGQRVIISCTGSSSNIGAGYDVHWYQQLPGTAPKLLVSSSSH |
| | | | RPSGVPDRESASKSGTSASLAITGLQAEDEADYFCQSYDSSLSGPVVFGGGTKLT |
| | | | VL |

TABLE 3
CDR HEAVY CHAIN SEQUENCES

| | | SEQ ID | | SEQ ID | | SEQ ID |
|---|---|---|---|---|---|---|
| Clone | CDRH1 | NO: | CDRH2 | NO: | CDRH3 | NO: |
| 2E6 | Ggaggcatc | 109 | gtcatccct | 110 | gcgtcttacccctatggcaaa | 111 |
| | ctcagtagt | | gtctttggt | | tactactttgactac | |
| | catgtt | | tcaaca | | | |
| | GGILSSHV | 112 | VIPVFGST | 113 | ASYPYGKYYFDY | 114 |
| 4E9 | ggtgggtcc | 115 | atcaatcac | 116 | gcgagcagcggggaaagcagc | 117 |
| | ccaagggga | | agtggaacc | | agacggggctacttctactat | |
| | tactac | | acc | | tacggtatggacgtc | |
| | GGSPRGYY | 118 | INHSGTT | 119 | ASSGESSRRGYFYYYGMDV | 120 |
| 4G8 | gaaggcacc | 121 | atcatccct | 122 | gcgagagacggcggggggagc | 123 |
| | ttcagcagc | | atgtttgct | | ttttgtggtggtgattgttat | |
| | tgtgct | | acaaca | | ttcgactta | |
| | EGTESSCA | 124 | IIPMFATT | 125 | ARDGGGSFCGGDCYFDL | 126 |
| 7G12 | ggttacacc | 127 | atcagcact | 128 | gcgaggggacgagatagtecg | 129 |
| | tttactaat | | tacgatggt | | gaccac | |
| | tatggt | | gccaca | | | |
| | GYTFTNYG | 130 | ISTYDGAT | 131 | ARGRDSPCH | 132 |
| 11H12 | ggattcgcc | 133 | attaatagt | 134 | gcgcgagattgggggacaaca | 135 |
| | tttagcggc | | aacggtctt | | ttggtaacttttgacctc | |
| | tactac | | accatc | | | |
| | GFAFSGYY | 136 | INSNGLTI | 137 | ARDWGTTLVTEDL | 138 |
| 12F9 | ggattcagc | 139 | attagtgga | 140 | gcgaaagattactgtggcagt | 141 |
| | ttcagtgac | | tccagtgcc | | ggcgcctgctacactgeggac | |
| | tactac | | tacaca | | cctggcttcttccatcaa | |
| | GFSFSDYY | 142 | ISGSSAYT | 143 | AKDYCGSGACYTADPGFFHQ | 144 |
| 14C10 | ggattcagt | 145 | attactggc | 146 | gcgagagatacgtacggtgac | 147 |
| | ttcagtaat | | agtagtagt | | accgatgatgcttttgatatt | |
| | tatcga | | tacatc | | | |
| | GFSFSNYR | 148 | ITGSSSYI | 149 | ARDTYGDIDDAFDI | 15C |
| 1A5 | ggattcacc | 151 | atatcatat | 152 | gcgaaagcaatggatgatagt | 153 |
| | ttcagtacc | | gatggaact | | agtggttattattgtcctgat | |
| | tatgtc | | aataaa | | tac | |
| | GFTFSTYV | 154 | ISYDGTNK | 155 | AKAMDDSSGYYCPDY | 156 |
| 5H1 | ggattcacc | 157 | atcacacat | 158 | gcgaaagacggcccctatgct | 159 |
| | ttcagtaaa | | gat ggaggt | | taccgttgggggggtttcttg | |
| | tatggc | | gataaa | | gacgac | |
| | GFTFSKYG | 160 | ITHDGGDK | 161 | AKDGPYAYRWGGFLDD | 162 |
| 9C1 | gggttcacc | 163 | atcggagct | 164 | gcgaaacagaaacgagaatgc | 165 |
| | tttagcgac | | ggtggttct | | agtggctggtgcaaaggatgg | |
| | tttgcc | | aacaca | | ggtttggacgtc | |
| | GFTFSDFA | 166 | IGAGGSNT | 167 | AKQKRECSGWCKGWGLDV | 168 |
| 10D5 | ggattcacc | 169 | attggaact | 170 | gcgagagtaatggtagtaggg | 171 |
| | ttcagttcg | | ggtagtaac | | ctttactactactacatggac | |
| | tctatg | | tacaaa | | atc | |
| | GFTESSSM | 172 | IGTGSNYK | 173 | ARVMVVGLYYYYMDI | 174 |
| 11G1 | ggatacagg | 175 | atctatccg | 176 | gcgagagaagtatatgtggct | 177 |
| | ttcaacact | | ggtgactcg | | tcgactgatagtgactattac | |
| | tattgg | | gatacg | | ggtatggacgtc | |
| | GYRFNTYW | 178 | IYPGDSDT | 179 | AREVYVASTDSDYYGMDV | 180 |
| 14B2 | gggttcagc | 181 | gtctataaa | 182 | gtgcgacatggcgatggttgg | 183 |
| | gtcagtacg | | gatggtgac | | aattacgtcgactcc | |
| | aggttc | | acc | | | |
| | GFSVSTRF | 184 | VYKDGDT | 185 | VRHGDGWNYVDS | 186 |
| 12C2 | gggttcacc | 187 | atcggagct | 188 | gcgaaacagaaacgagaatgc | 189 |
| | tttagcgac | | ggtggttct | | agtggctggtgcaaaggatgg | |
| | tttgcc | | aacaca | | ggtttggacgtc | |
| | GFTESDFA | 190 | IGAGGSNT | 191 | AKQKRECSGWCKGWGLDV | 192 |
| 18D4 | ggt gggacg | 193 | gtcagtcat | 194 | gggagaggtcggggttacact | 195 |
| | ttcagtgat | | act gggagg | | tggaatgat | |
| | tacttc | | acc | | | |
| | GGTFSDYF | 196 | VSHTGRT | 197 | GRGRGYTWND | 198 |
| 30A10 | ggattcagt | 199 | attagtact | 200 | gcgagagatggacctccgata | 201 |
| | ttcagtagc | | agtggtaat | | gtagcagctggcctgatctac | |
| | tatagc | | tacata | | tactactacatgggcgtc | |
| | GFSFSSYS | 202 | ISISGNYI | 203 | ARDGPPIVAAGLIYYYYMGV | 204 |
| 18H7 | ggtgactcc | 205 | attgatcac | 206 | gcgagagaccagaagtatgag | 207 |
| | atcgtgact | | agtggaact | | ggagataattggttcgacccc | |
| | ggtaagtac | | acc | | | |
| | tac | | | | | |
| | GDSIVTGK | 208 | IDHSGTT | 209 | ARDQKYEGDNWEDP | 210 |
| | YY | | | | | |
| 4E1 | ggatacacc | 211 | atcaaccct | 212 | gcgaccctctattactatgat | 213 |
| | ttcaccaga | | agt gatggt | | gatagtggttattacactgac | |
| | tactat | | cgcaca | | tac | |
| | GYTFTRYY | 214 | INP SDGRT | 215 | ATLYYYDDSGYYTDY | 216 |
| 12D4 | ggatacacc | 217 | atcacacct | 218 | gctgtggcttatcactatgat | 219 |
| | ttcacctac | | ttcaatgat | | agtagtggtccgtactttgac | |
| | cgctac | | aacacc | | cac | |
| | GYTFTYRY | 220 | ITPENDNT | 221 | AVAYHYDSSGPYFDH | 222 |

TABLE 4
CDR LIGHT CHAIN SEQUENCES

| | | SEQ ID | | SEQ ID | | SEQ ID |
|---|---|---|---|---|---|---|
| Clone | CDRL1 | NO: | CDRL2 | NO: | CDRL3 | NO: |
| 2E6 | agctccaacat | 223 | gacaatttt | 224 | ggagcatgggatctcggcctc | 225 |
| | tggcagtaatt | | | | aacgctggggtg | |
| | at | | | | | |
| | SSNIGSNY | 226 | DNE | 227 | GAWDLGINAGV | 228 |
| 4E9 | agctccaacaa | 229 | agaaacagt | 230 | gcagcgtgggatgacatcctg | 231 |
| | cgaaaattatt | | | | agtggtccgagggtg | |
| | at | | | | | |
| | SSNNENYY | 232 | RNS | 233 | AAWDDISSGPRV | 234 |
| 4G8 | cagagtgttaa | 235 | ggtgcatct | 236 | cagcagtataataactggcct | 237 |
| | cagcaac | | | | cccact | |
| | QSVNSN | 238 | GAS | 239 | QQYNNWPPT | 240 |
| 7G12 | cagagtattaa | 241 | aaggcgtct | 242 | caacagtatgatcattttccg | 243 |
| | taggtgg | | | | cacact | |
| | QSINRW | 244 | KAS | 245 | QQYDHFPHT | 246 |
| 11H12 | agtggccacag | 247 | cttgagggtagtg | 248 | ttcacctgggacagcgactcc | 249 |
| | aggctacaac | | actcctac | | cgcgtc | |
| | SGHRGYN | 250 | LEGSDSY | 251 | FTWDSDSRV | 252 |
| 12F9 | aacattggaag | 253 | tatgatagc | 254 | caggtgtgggacaatactaat | 255 |
| | taaaagt | | | | gatcatccctcttatgtc | |
| | NIGSKS | 256 | YDS | 257 | QVWDNTNDHPSYV | 258 |
| 14C10 | gcattggcaaa | 259 | aaagacatt | 260 | cacgtagttgacaggactgag | 261 |
| | ggaatat | | | | acgtatcgaagtgtc | |
| | ALAKEY | 262 | KDI | 263 | HVVDRIETYRSV | 264 |
| 1A5 | agcctcagaaa | 265 | ggt caaaac | 266 | aactcccgggacagcagtggt | 267 |
| | ctatttt | | | | aaccatctttatgtc | |
| | SLRNYE | 268 | GQN | 269 | NSRDSSGNHLYV | 270 |
| 5H1 | gagagtattgg | 271 | ggagcctcc | 272 | cagcagtatcatagctggccc | 273 |
| | cagcagt | | | | ccgctcact | |
| | ESIGSS | 274 | GAS | 275 | QQYHSWPPLI | 276 |
| 9C1 | cagagectect | 277 | ttggcttct | 278 | atgcaagctctacaaactect | 279 |
| | ggatagtaagg | | | | tacact | |
| | gatccacctat | | | | | |
| | QSLLDSKGSTY | 280 | LAS | 281 | MQALQTPYT | 282 |
| 10D5 | cagaatgttag | 283 | ggtgcgtcc | 284 | caactgtacggtacctcacca | 285 |
| | tagcaacttc | | | | acgtggacg | |
| | QNVSSNF | 286 | GAS | 287 | QLYGTSPTWT | 288 |
| 11G1 | agcaatgatgt | 289 | gagtccagt | 290 | tgttcatatgcaggtggtaac | 291 |
| | tgggcgttctg | | | | acttatgtc | |
| | acctt | | | | | |
| | SNDVGRSDL | 292 | ESS | 293 | CSYAGGNTYV | 294 |
| 14B2 | cacagtcttag | 295 | gatgcatcc | 296 | cagcaatataataactggccg | 297 |
| | tagtcac | | | | ctcact | |
| | HSLSSH | 298 | DAS | 299 | QQYNNWPLT | 300 |
| 12C2 | cagagcctcct | 301 | ttggcttct | 302 | atgcaagctctacaaactcct | 303 |
| | ggatagtaagg | | | | tacact | |
| | gatccacctat | | | | | |
| | QSLLDSKGSTY | 304 | LAS | 305 | MQALQTPYT | 306 |
| 18D4 | cagagcattag | 307 | gctgcaacc | 308 | cagcagagctacagtggccct | 309 |
| | aacctct | | | | ccaact | |
| | QSIRTS | 310 | AAT | 311 | QQSYSGPPT | 312 |
| 30A10 | cagggcattag | 313 | gctgcatcc | 314 | caacaccttaatagttaccgg | 315 |
| | cagttat | | | | act | |
| | QGISSY | 316 | AAS | 317 | QHLNSYRT | 318 |
| 18H7 | agctccaacgt | 319 | gggactaat | 320 | tcggcgtgggatgagagcctg | 321 |
| | cgcatataata | | | | agtggtccggtc | |
| | gt | | | | | |
| | SSNVAYNS | 322 | GTN | 323 | SAWDESLSGPV | 324 |
| 4E1 | cagagtgtcag | 325 | ggtgcatca | 326 | cagcagtatggtaactcaccg | 327 |
| | caacaactac | | | | tggacg | |
| | QSVSNNY | 328 | GAS | 329 | QQYGNSPWT | 330 |
| 12D4 | agctccaacat | 331 | agtagcagc | 332 | cagtcgtatgacagcagtctg | 333 |
| | cggggcaggtt | | | | agtggtcccgtggta | |
| | atgat | | | | | |
| | SSNIGAGYD | 334 | SSS | 335 | QSYDSSLSGPVV | 336 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, NY, 1988.
Abbondanzo et al., Am. J. Pediatr. Hematol. Oncol. 12(4): 480-489, 1990.
Allred et al., Arch. Surg. 125(1), 107-113: 1990.
Atherton et al., Biol. of Reproduction 32: 155-171, 1985.
Barzon et al., Euro Surveill. 11: 21(32), 2016.
Beltramello et al., Cell Host Microbe 8: 271-283, 2010.
Brown et al., J. Immunol. Meth. 130(1): 111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm. 74(2):4 25-433, 1977.
De Jager et al., Semin. Nucl. Med. 23(2): 165-179, 1993.
Dholakia et al., J. Biol. Chem. 264: 20638-20642, 1989.
Diamond et al., J. Virol. 77: 2578-2586, 2003.
Doolittle and Ben-Zeev, Methods Mol. Biol. 109: 215-237, 1999.
Duffy et al., N. Engl. J. Med. 360: 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet. 3: 231-236, 1977.
Gornet et al., Semin Reprod Med. 34(5): 285-292, 2016.
Gulbis and Galand, Hum. Pathol. 24(12): 1271-1285, 1993.
Halfon et al., PLoS ONE 5(5): e10569, 2010
Hessell et al., Nature 449: 101-104, 2007.
Khatoon et al., Ann. of Neurology 26: 210-219, 1989.
King et al., J. Biol. Chem. 269: 10210-10218, 1989.
Kohler and Milstein, Eur. J. Immunol. 6: 511-519, 1976.
Kohler and Milstein, Nature 256: 495-497, 1975.
Kyte and Doolittle, J. Mol. Biol. 157(1): 105-132, 1982.
Mansuy et al., Lancet Infect Dis. 16(10): 1106-7, 2016.
Nakamura et al., In: Enzyme Immunoassays: Heterogeneous and Homogeneous Systems, Chapter 27, 1987.
O'Shannessy et al., J. Immun. Meth. 99: 153-161, 1987.
Persic et al., Gene 187: 1, 1997
Potter and Haley, Meth. Enzymol. 91: 613-633, 1983.
Purpura et al., Lancet Infect Dis. 16(10): 1107-8, 2016.
Remington's Pharmaceutical Sciences, 15th Ed., 3: 624-652, 1990.
Smith et al., Nat Protoc. 2009; 4(3):372-384.
Sterner et al., ACS Chem Biol. 2016; 11(7):1773-1783.
Tang et al., J. Biol. Chem., 271: 28324-28330, 1996.
Wawrzynczak & Thorpe, In: Immunoconjugates, Antibody Conuugates In Radioimaging And Therapy Of Cancer, Vogel (Ed.), NY, Oxford University Press, 28, 1987.
Wurth et al., JCI Insight. 2018;3(20):e123387.
Yu et al., J Immunol Methods 336: 142-151, 2008.
Jarrett, E. & Bazin, H., Nature 251, 613-614 (1974).
Bethony et al., FASEB J. 19, 1743-1745 (2005).
Pritchard, D. I., Quinnell, R. J., & E. A. Walsh, Parasite Immunol. 17, 71-75 (1995).
Faulkner, H., et al. Age- and infection intensity-dependent cytokine and antibody production in human trichuriasis: The importance of IgE. J. Infect. Dis.185, 665-72 (2002).
Turner et al., Microbes Infect. 7, 990-96 (2005).
McSharry, C., Xia, Y., Holland C. V. & Kennedy M W., Infect. Immun. 67, 484-489 (1999).
Fitzsimmons et al., Infect. Immun. 80, 4264-4270 (2012).
Pinot de Moira et al., PLoS Neg. Trop. Dis. 4, e820 (2010).
Jiz et al., Infect. Immun. 77, 2051-2058 (2009).
Dunne et al., Eur. J. Immunol. 22, 1483-1494 (1992).
Dunne et al., Parasite Immunol. 19, 79-89 (1997).
Hagan, P., Blumenthal, U. J., Dunn, D., Simpson, A. J. & Wilkins, H. A., Nature 349, 243-245 (1991).
Rihet et al., Eur. J. Immunol. 21, 2679-2686 (1991).
Jarrett, E., Mackenzie, S. & Bennich, H., Nature 283, 302-303 (1980).
Sereda, M. J., Hartmann, S. & Lucius, R., Trends Parasitol. 24, 272-278 (2008).
Gazzinelli-Guimaraes, P. H., et al., PLoS Pathog. 17, e1009337 (2021).
Santiago et al., J. Allergy Clin. Immunol. 127, 479-486 (2011).
Nutman, T. B. Parasite Immonol. 37,304-313 (2015).
Ottesen, E. A. & Nutman TB. Annu Rev Med. 43, 417-424 (1992).
Wurth, et al., JCI Insight 3, e123387 (2018).
Croote, D., Darmanis, S., Nadeau, K. C. & Quake, S. R., Science 362, 1306-1309 (2018).
Hewitson et al., Mol. Biochem. Parasitol. 160, 8-21 (2008).
Geary et al., Dirofilaria immitis. Parasit. Vectors 5, 10.1186/1756-3305-5-140 (2012).
Cantacessi et al., Infect. Genet. Evol. 10, 1199-1207 (2010).
Sonnhammer, E. L. & Durbin R., Genomics 46, 200-216 (1997).
Lin et al., New Phytol. 209, 1159-1173 (2016).
Dissanayake, S., Xu, M. & Piessens, W. F., Mol. Biochem. Parasitol. 56, 269-277 (1992).
Rao et al., Mol. Biochem. Parasitol. 107, 71-80 (2000).
Rahman, A. R., Hwen-Yee, C. & Noordin, R., Filaria J. 6,1-7 (2007).
Garcia-Mayoral et al., PLoS Neg. Trop. Dis. 8, e2735-e2735 (2014).
Naotoshi et al., Infect. Immun. 71, 5314-5323 (2003).
Frank et al., Mol. Biochem. Parasitol. 98, 297-302 (1999).
Pastrana et al., Infect. Immun. 66, 5955-5963 (1998).
Tweedie et al., Exp. Parasitol. 76, 156-164 (1993).
Sotoet al., Nature 566, 398-402 (2019).
Osterfeld et al., J. Allergy Clin. Immunol. 125, 496-476 (2010).
Smith, S. A. & Crowe, J. E. Jr., Microbiol Spectr. 3, AID-0027-2014 (2015).
Smith et al., J. Virol. 86, 2665-2675 (2012).
Smith et al., Nature Protocols 4, 372-384 (2009).
Lobstein et al., Microb. Cell Fact. 8, 10.1186/1475-2859-11-56 (2012).
Dombrowicz et al., J. Immunol. 157, 1645-1651 (1996).

The following listing of claims replaces all previous listings or versions thereof:

1. A method of detecting a parasitic worm antigen in a sample comprising:
(a) contacting a sample with an antibody or antibody fragment comprising clone-paired heavy and light chain CDR sequences from Tables 3 and 4, respectively; and
(b) detecting a parasitic worm antigen in said sample by binding of said antibody or antibody fragment to a parasitic worm antigen in said sample.

2. The method of claim 1, wherein said sample is a biologic fluid, such has a biologic culture, media or culture supernatant, blood, sputum, tears, saliva, mucous or serum, semen, cervical or vaginal secretions, amniotic fluid, placental tissues, urine, exudate, transudate, tissue scrapings or feces, a food or tissue sample, a drug formulation, a biologic therapeutic formulation or a vaccine stock.

3. The method of claim 1, wherein detection comprises ELISA, RIA, lateral flow assay or Western blot.

4. The method of claim 1, further comprising performing steps (a) and (b) a second time and determining a change in helminth antigen levels as compared to the first assay.

5. The method of claim 1, wherein the antibody or antibody fragment is encoded by clone-paired variable sequences as set forth in Table 1.

6. The method of claim 1, wherein said antibody or antibody fragment is encoded by light and heavy chain variable sequences having 70%, 80%, or 90% identity to clone-paired variable sequences as set forth in Table 1.

7. The method of claim 1, wherein said antibody or antibody fragment is encoded by light and heavy chain variable sequences having 95% identity to clone-paired sequences as set forth in Table 1.

8. The method of claim 1, wherein said antibody or antibody fragment comprises light and heavy chain variable sequences according to clone-paired sequences from Table 2.

9. The method of claim 1, wherein said antibody or antibody fragment comprises light and heavy chain variable sequences having 70%, 80% or 90% identity to clone-paired sequences from Table 2.

10. The method of claim 1, wherein said antibody or antibody fragment comprises light and heavy chain variable sequences having 95% identity to clone-paired sequences from Table 2.

11. The method of claim 1, wherein the antibody fragment is a recombinant scFv (single chain fragment variable) antibody, Fab fragment, F(ab′)₂ fragment, or Fv fragment.

12. A method of detecting IgE anti-parasitic worm antigen antibodies in a sample comprising:
(a) contacting a sample suspected of containing an IgE anti-parasitic worm antigen antibody with a parasitic worm antigen in a competitive assay with an antibody or antibody fragment comprising clone-paired heavy and light chain CDR sequences from Tables 3 and 4, respectively; and
(b) detecting competition for binding of parasitic worm antigen by a suspected IgE anti-parasitic worm antigen in said sample by an antibody or antibody fragment comprising clone-paired heavy and light chain CDR sequences from Tables 3 and 4, respectively.

13. (canceled)

14. A method of preventing or treating parasitic worm infection in a subject comprising delivering to said subject an IgE antibody or antibody fragment comprising clone-paired heavy and light chain CDR sequences from Tables 3 and 4, respectively.

15-25. (canceled)

26. A monoclonal antibody or antigen-binding fragment thereof,
wherein:
(a) the antibody or antigen-binding fragment thereof comprises clone-paired heavy and light chain CDR sequences from Tables 3 and 4, respectively, or
(b) the antibody or antigen-binding fragment binds to a parasitic worm antigen selected from SXP-1, MIF, 24kDaSPro, Gpl5/400, TTR 4, TTR 32, TTR 61, TTR 76, TTR 62, TTR 79, TTR 07, TTR 08, TTR 16, TTR 90, TTR 77, TTR 47, TTR 41, TTR 95, TTR 05, or a homolog thereof.

27-61. (canceled)

62. A vaccine composition comprising one or more parasitic worm antigens selected from SXP-1, MIF, 24kDaSPro, Gpl5/400, TTR_4, TTR_32, TTR_61, TTR_76, TTR_62, TTR_79, TTR_07, TTR_08, TTR_16, TTR_90, TTR_77, TTR_47, TTR_41, TTR_95, TTR_05, or a homolog thereof.

63. A method of immunizing a subject against a parasitic worm infection comprising administering to said subject a vaccine composition according to claim 62.


SEQUENCE LISTING (353 sequences)

SEQ ID NO: 1 - protein, 17 aa, Homo sapiens
CATLYYYDDSGYYTDYW

SEQ ID NO: 2 - protein, 17 aa, Homo sapiens
CAVAYHYDSSGPYFDHW

SEQ ID NO: 3 - protein, 18 aa, Homo sapiens
CAKDGPYAYRWGGFLDDW

SEQ ID NO: 4 - protein, 20 aa, Homo sapiens
CAKQKRECSGWCKGWGLDVW

SEQ ID NO: 5 - protein, 16 aa, Homo sapiens
CARDTYGDTDDAFDIW

SEQ ID NO: 6 - protein, 11 aa, Homo sapiens
CARGRDSPDHW

SEQ ID NO: 7 - protein, 17 aa, Homo sapiens
CAKAMDDSSGYYCPDYW

SEQ ID NO: 8 - protein, 22 aa, Homo sapiens
CAKDYCGSGACYTADPGFFHQW

SEQ ID NO: 9 - protein, 19 aa, Homo sapiens
CAREVYVASTDSDYYGMDV

SEQ ID NO: 10 - protein, 14 aa, Homo sapiens
CVRHGDGWNYVDSW

SEQ ID NO: 11 - protein, 17 aa, Homo sapiens
CARVMVVGLYYYYMDIW

SEQ ID NO: 12 - protein, 12 aa, Homo sapiens
CGRGRGYTWNDW

SEQ ID NO: 13 - protein, 22 aa, Homo sapiens
CARDGPPIVAAGLIYYYYMGVW

SEQ ID NO: 14 - protein, 21 aa, Homo sapiens
CASSGESSRRGYFYYYGMDVW

SEQ ID NO: 15 - protein, 14 aa, Homo sapiens
CASYPYGKYYFDYW

SEQ ID NO: 16 - protein, 15 aa, Homo sapiens
CARDWGTTLVTFDLW

SEQ ID NO: 17 - protein, 11 aa, Homo sapiens [sequence omitted]

SEQ ID NO: 18 - protein, 14 aa, Homo sapiens [sequence omitted]

SEQ ID NO: 19 - protein, 12 aa, Homo sapiens [sequence omitted]

SEQ ID NO: 20 - protein, 11 aa, Homo sapiens [sequence omitted]

SEQ ID NO: 21 - protein, 14 aa, Homo sapiens
CHVVDRTETYRSVF

SEQ ID NO: 22 - protein, 11 aa, Homo sapiens
CQQYDHFPHTF

SEQ ID NO: 23 - protein, 14 aa, Homo sapiens [sequence omitted]

SEQ ID NO: 24 - protein, 15 aa, Homo sapiens
CQVWDNTNDHPSYVF

SEQ ID NO: 25 - protein, 12 aa, Homo sapiens [sequence omitted]

SEQ ID NO: 26 - protein, 11 aa, Homo sapiens [sequence omitted]

SEQ ID NO: 27 - protein, 12 aa, Homo sapiens
CQLYGTSPTWTF

SEQ ID NO: 28 - protein, 11 aa, Homo sapiens
CQQSYSGPPTF

SEQ ID NO: 29 - protein, 10 aa, Homo sapiens
CQHLNSYRTF

SEQ ID NO: 30 - protein, 14 aa, Homo sapiens
CAAWDDILSGPRVF

SEQ ID NO: 31 - protein, 13 aa, Homo sapiens
CGAWDLGLNAGVF

SEQ ID NO: 32 - protein, 11 aa, Homo sapiens
CFTWDSDSRVF

SEQ ID NO: 33 - DNA, 358 bp, Homo sapiens
  source 1..358: mol_type = other DNA
caggtgcagctggtgcagtctggggctgaggtgaagaagcctgggtcctcgctgaaggtctcctgcaaggcttctggaggcatcctcagtagtcatgttatcagctgggtgcgacaggccccgggacaaggccttgagtggatgggaggggtcatccctgtctttggttcaacaacctacgcaccgaagttccaggacagactcacagttaccgcggacgaatccacgaatacagcctacatggacctgagcagcctgagctctgaggacacggccatttattactgtgcgtcttacccctatggcaaatactactttgactactggggccagggaaccctggtcaccgtctcctcag

SEQ ID NO: 34 - DNA, 331 bp, Homo sapiens
  source 1..331: mol_type = other DNA
cagtctgtgttgactcagccgccctcagtgtctgcggccccaggacagaaggtcaccatctcctgctctggaaacagctccaacattggcagtaattatgtatcctggtaccagcaggtcccaggaacagcccccaaactcctcatttatgacaattttaagcgaccctcagacattcctgaccgattctctggctccaagtctggcacgtcagccaccctgggcatcaccggactccagcctggggacgaggccgattattactgcggagcatgggatctcggcctcaacgctggggtgttcggcggagggaccaaactgaccgtcctag

SEQ ID NO: 35 - DNA, 375 bp, Homo sapiens
  source 1..375: mol_type = other DNA
caggtgcagctacaacagtggggcgcaggcctgttgaagccttcggagaccctgtccctcacgtgcgctgtctatggtgggtccccaaggggatactactggagctggatccgccagcccccagggaagggactggaatggattggggaaatcaatcacagtggaaccaccaactacaacccgtccctcacgagtcgagtcaccatatcagcagacacgtccaagagccaattttccctgaggctgacgtctgtgaccgccgcggacacggctatatatttctgtgcgagcagcggggaaagcagcagacggggctacttctactattacggtatggacgtctggggccaagggaccacggtcaccgtctcctca

SEQ ID NO: 36 - DNA, 334 bp, Homo sapiens
  source 1..334: mol_type = other DNA
cagtctgtgctgactcagccaccctcagcgtctgggacccccgggcagacggtcaccatctcttgttctggcatcagctccaacaacgaaaattattatgtttattggtaccagcagctcccaggagcggcccccaggctcctcatctatagaaacagtcagcggccctcaggggtccctgaccgattctcaggctccaagtctgacacctcagcctccctggccatcagcgggctccggtccgaggatgaggctgattattactgtgcagcgtgggatgacatcctgagtggtccgagggtgttcggcggagggaccaagttgaccgtcctac

SEQ ID NO: 37 - DNA, 373 bp, Homo sapiens
  source 1..373: mol_type = other DNA
caggtgcagttggtgcagtctggggctgaggtgaagaagcctgggtcctcagtgaaggtctcctgcaaggcttctgaaggcaccttcagcagctgtgctatcagctgggtgcgacaggcccctggacaagggcttgagtggatgggagggatcatccctatgtttgctacaacacgctacgcccagaagttccaggacagagtcaccattaccacggacgaatcgacgaacacagcctacatggagctgagcagcctgagatctgaggacacggccatttattactgtgcgagagacggcggggggagcttttgtggtggtgattgttatttcgacttatggggccagggaaccctggtcaccgtctcctcag

SEQ ID NO: 38 - DNA, 322 bp, Homo sapiens
  source 1..322: mol_type = other DNA
gaaatagtgatgacgcagtctccagccaccctgtctgtgtctccaggggaaagagccaccctctcctgcagggccagtcagagtgttaacagcaacttggcctggtatcagcagaaacctggccaggctcccaggctcctcatctttggtgcatctatcagggccactggtatcccagccaggttcagtggcagtgggtctgggacggagttcactctcaccatcagcagcctgcaatctgaagattttgcagtgtattactgtcagcagtataataactggcctcccactttcggccctgggaccaaagtggatatcaaac

SEQ ID NO: 39 - DNA, 348 bp, Homo sapiens
  source 1..348: mol_type = other DNA
caggttcagctggtgcagtctggagttgacgtgaagaagcctggggcctcagtgaaactctcctgcaagacttctggttacacctttactaattatggtattacttgggtgcgacaggcccctggacaagggcttgagtggatgggctggatcagcacttacgatggtgccacaaactatagccagaatctccagggcagaatcatcatgaccactgacacatccaagaggacagcctatctgcagatgaggagtctgagatctgacgacacggccgtctattactgtgcgaggggacgagatagtccggaccactggggccagggaaccctggtcaccgtctcctca

SEQ ID NO: 40 - DNA, 321 bp, Homo sapiens
  source 1..321: mol_type = other DNA
gacatccagatgacccagtctccttccaccctgtctgcatctgtaggagacagagtcaccatcacttgccgggccagtcagagtattaataggtggttggcctggtatcagcagaaaccagggacagcccctaaactcctcatctttaaggcgtctactctagacagtggtgtcccagcgaggttcagcggcactggatctgagacagaattctctctgaccatcaacagcctgcagcctgatgattttgcaacttattactgccaacagtatgatcattttccgcacacttttggcccggggaccaaactggacatcaaa

SEQ ID NO: 41 - DNA, 360 bp, Homo sapiens
  source 1..360: mol_type = other DNA
caggtccaactggtggagtctgggggagacttggtcacgcctggagggtccctgagactgtcgtgtgcagcctcgggattcgcctttagcggctactacatgagttggatccgccaggctccagggaaggggctggaatggatctcatacattaatagtaacggtcttaccatctcctacgcggactctgtgaagggccgattcaccgtctccagggacaatgccaagaactcactgtttctgcaaatgagctccctgagagccgaggacacggccatatattactgtgcgcgagattgggggacaacattggtaacttttgacctctggggccagggaaccctggtcaccgtctcctca

SEQ ID NO: 42 - DNA, 318 bp, Homo sapiens
  source 1..318: mol_type = other DNA
cagcctgtgctgactcaatcatcctctgcctctgcttccatgggatcctcggccaagctcacctgtactctgagtagtggccacagaggctacaacatcgcttggcttcagcagcatccagggaaggcccctctctatttgacgaatcttgagggtagtgactcctacaagaacgatcgcctcacagtctccagctctggggctgaccgctacctcaccatctccaacctccagcctgaagatgaggctacctattactgcttcacctgggacagcgactcccgcgtcttcggcggggggacgcacctgaccgtcctg

SEQ ID NO: 43 - DNA, 357 bp, Homo sapiens
  source 1..357: mol_type = other DNA
caggtgcagctggtggagtctgggggagacttggtcaagcctggagggtccctgagaatctcctgtgcagcctctggattcagcttcagtgactactacatgagttggatccgccaggctccagggaaggggcttgaatgggttgcgtatattagtggatccagtgcctacacaagctacgcggactctgtgaagggccgcttctccatctccagagacaacgccaacaactcactctttctacaaatgaacagcctgagagccgaggacacggctacatatttctgtgcgaaagattactgtggcagtggcgcctgctacactgcggaccctggcttcttccatcaatgggccagg

SEQ ID NO: 44 - DNA, 331 bp, Homo sapiens
  source 1..331: mol_type = other DNA
tcctatgttctgactcagccgccctcagtgtcggtggccccaggaaagacggccacgatttcctgtgggggaaacaacattggaagtaaaagtgtgcactggtatcagcagaagccaggccaggcccctatagtggtcatctattatgatagcgaccggccctcagggatccctgagcgattttctggaatcaattctgggaacacggccaccctgaccatcagcagggtcgaagccggggatgaggccgactattattgtcaggtgtgggacaatactaatgatcatccctcttatgtcttcggagctgggaccaaggtcaccntcctag

SEQ ID NO: 45 - DNA, 364 bp, Homo sapiens
  source 1..364: mol_type = other DNA
gaggtgcagctggtgcagtcggggggagacctggtcaagcctggggggtccctgagactctcctgtgcagcctctggattcagtttcagtaattatcgaatgaactgggtccgccaggctccagggaaggggctggagtgggtctcatccattactggcagtagtagttacatctactacgcagactccgtgcagggccgattcaccatctccagagacaacgccaagaactcaatgtctctgcaaatgaagagcctgagagtcgaggacacggctgtgtattactgtgcgagagatacgtacggtgacaccgatgatgcttttgatatttggggccaagggacaatggtcaccgtctcctcag

SEQ ID NO: 46 - DNA, 328 bp, Homo sapiens
  source 1..328: mol_type = other DNA
tcccttgagttgatacagccaccctcggtggcggtgtccccaggacagacggccaggatcagctgctctggaaatgcattggcaaaggaatatgctaattggtaccagcagaggccaggccaggcccctgttttggtgatttttaaagacattgagaggcccaccgggattcctgagcgattctctggctccagctcagggacaacactcacgttgaccatcagtggagtccaggcagaagacgaggctaattattactgtcacgtagttgacaggactgagacgtatcgaagtgtcttcggcggagggaccaagctgaccgtcctag

SEQ ID NO: 47 - DNA, 344 bp, Homo sapiens
  source 1..344: mol_type = other DNA
gaagtgcagctggtggagtctgggggaggcgtggtccagcctgggaggtccctgagactctcctgtgcaggctctggattcaccttcagtacctatgtcatgcactgggtccgccaggctccaggcaaggggctggagtgggtggcagttatatcatatgatggaactaataaatactatgcagactccatgaagggccgattcaccatctccagagacaattccaagaacacgctgtatctgcaactgaaccgcctgagagctgaggacacggctgtgtattactgtgcgaaagcaatggatgatagtagtggttattattgtcctgattactgggccangna

SEQ ID NO: 48 - DNA, 328 bp, Homo sapiens
  source 1..328: mol_type = other DNA
tcttctgagctgactcaggaccctgctgtgtctgtggccttgggacagacagtcaagatcacatgccaaggagacagcctcagaaactattttgcaaactggtaccagcagaagccaggacaggcccctgttcttgtcatctatggtcaaaacaaccggccctcagggatcccagaccgattctctggctccacctcaggaaacacaggttccttgaccatcactggggctcaggcggaagatgaggctgactattactgtaactcccgggacagcagtggtaaccatctttatgtcttcggaactgggaccaaggtcaccgtcctag

SEQ ID NO: 49 - DNA, 370 bp, Homo sapiens
  source 1..370: mol_type = other DNA
caggtgcagctggtgcagtcgggggggggcgtggtccagcctgggaggtcattgagactctcatgtaatgcctctggattcaccttcagtaaatatggcatgcactgggtccgccaggctccaggcaaggggctcgagtgggtggcagttatcacacatgatggaggtgataaacaccatgcagactccgtgaagggccgatttagcatctccagagacaattccaaggacatcctgtatctgcaaatggacagcctgagaactgaggacacggctatttattactgtgcgaaagacggcccctatgcttaccgttgggggggtttcttggacgactggggccagggaactccggtcaccgtctcctcag

SEQ ID NO: 50 - DNA, 325 bp, Homo sapiens
  source 1..325: mol_type = other DNA
gaaatagtgatgacgcagtctccagtcaccctgtctgtctctccaggggagagagtcaccctctcctgcagggccagtgagagtattggcagcagtttagcctggtaccaacaaaaacctggccaggctcccaggctcctcatctatggagcctccaccaggaccactggtatcccagccagattcagtggcagtgggtctgggacagagttcactctcgccatcagcagcctgcagtctgaagattatgcagtttatttctgtcagcagtatcatagctggcccccgctcactttcggcggagggaccaaggtggagatcaaac

SEQ ID NO: 51 - DNA, 375 bp, Homo sapiens
  source 1..375: mol_type = other DNA
gaggtgcagctgtcggagtctgggggaggcttggtacagccgggggggtccctgagactctcctgtgcagcctctgggttcacctttagcgactttgccatgagttgggtccgccaggctccagggaagggactggagtgggtctcagctatcggagctggtggttctaacacatacaacgcagactccgtgaagggccggttcaccatctccagggacaattccgcgaacacgctgtatttgcaaatgaatagcctgagagccgaggacacggccctatattactgtgcgaaacagaaacgagaatgcagtggctggtgcaaaggatggggtttggacgtctggggccaagggaccacggtcaccgtctcctca

SEQ ID NO: 52 - DNA, 337 bp, Homo sapiens
  source 1..337: mol_type = other DNA
gatattgtgatgactcagtctccactctccctgcccgtcacccctggagagccggcctccatctcctgcaagtctagtcagagcctcctggatagtaagggatccacctatttggattggtacctgcagaagccagggcagtctccacagctcctgatctatttggcttcttatcgggcctccggggtccctgacaggttcagtggcagtggatcaggcacagattttacactgaaaatcagcagagtggaggctgaggatgttggggtttattactgcatgcaagctctacaaactccttacacttttggccaggggaccaggctggagatcaaac

SEQ ID NO: 53 - DNA, 366 bp, Homo sapiens
  source 1..366: mol_type = other DNA
gaggtgcagctggtggagtcggggggaggcctggtcaagcctggggggtccctgagactctcctgtgcagcctctggattcaccttcagttcgtctatgatgacctgggtccgccaggctccagggaaggggctggagtgggtctcatctattggaactggtagtaactacaaacactacgcagactcactgaggggccgattcaccgtctcccgagacaacgccaagaactcactgtttctacaaatgaacagcctgagacccgaggacacggctgtctattactgtgcgagagtaatggtagtagggctttactactactacatggacatctggggcacagggaccacggtcaccgtctcctca

SEQ ID NO: 54 - DNA, 328 bp, Homo sapiens
  source 1..328: mol_type = other DNA
gaaattgtgttgacgcagtctccaggcaccctgtctttgtctccaggggaaagagccgccctctcctgcagggccagtcagaatgttagtagcaacttcttagcctggtaccagcagaaccctggccaggctcccaggctcctcttctatggtgcgtccatgagggccactggcatcccagacaggttcagtggcagtgggtctgggacagacttcactctcaccatcagtagactggagcctgaagattttgcagtgtattactgtcaactgtacggtacctcaccaacgtggacgttcggccaagggaccaaggtggagatcaaac

SEQ ID NO: 55 - DNA, 356 bp, Homo sapiens
  source 1..356: mol_type = other DNA
gaggtgcagctggtccagtctggaccagtgctaaaaaagccgggggagtctctgaagatctcctgtaggggctctggatacaggttcaacacttattgggtcgcctgggtgcgccagatgcccgggaaaggtctggagtggatgggaatgatctatccgggtgactcggatacgaaatatagtccgtccttccaaggccaagtcaccatctcagccgacaagtccagcaataccgcctacctacagtggagtagtctgaaggcctcggacaccgccatgtattattgtgcgagagaagtatatgtggcttcgactgatagtgactattacggtatggacgtcnnngcctaggacca

SEQ ID NO: 56 - DNA, 332 bp, Homo sapiens
  source 1..332: mol_type = other DNA
agtctgcncctgactcaacctgcctcgcgtgtctgggtctcctggacagtcgatcaccatctcctgcactggaaccagcaatgatgttgggcgttctgaccttgtcgcctggtaccaacaacaccccgacaaggcccccagactcattatttatgagtccagtaagcggccctcaggggtttctgcccgcttctctggctccnggtctggcatcacggcctccctgacaatctctgacctccaggctgaagacgaggctgactattactgctgttcatatgcaggtggtaacacttatgtcttcggcaccgcgaccggggtcaccgtcctag

SEQ ID NO: 57 - DNA, 334 bp, Homo sapiens
  source 1..334: mol_type = other DNA
gaggtgcagctggtggagactggaggaggcttgattcagccgggggggtccctgcgactctcctgtgcagcctctgggttcagcgtcagtacgaggttcatgagctgggtccgccaggctccaggtcagggactggagtgggtctcagtcgtctataaagatggtgacaccttcaactcggactccgtgaagggccgattcagcatctccagagacaattccaagaacacagtgtttcttcaaatgaacagactgagagtcgaagacactgccgtatacttctgtgtgcgacatggcgatggttggaattacgtcgactcctggggcctggaac

SEQ ID NO: 58 - DNA, 322 bp, Homo sapiens
  source 1..322: mol_type = other DNA
gaaatagtgatgacgcagtctccagccaccctgtctgtgtctccaggggaaggagccaccctctcctgcagggccagtcacagtcttagtagtcacttagcctggtaccagcaaaaacctggccaggctcccaggctcctaatatatgatgcatccgtcagggccactgatatcccagccaggttcagtggcagtgggtctgggacagagttcactctcaccatcagcagcctgcagtctgaagattttgcagtttactactgtcagcaatataataactggccgctcactttcggcggagggaccaagcngagatcaancg

SEQ ID NO: 59 - DNA, 375 bp, Homo sapiens
  source 1..375: mol_type = other DNA
gaggtgcagctgtcggagtctgggggaggcttggtacggccgggggggtccctgagactctcctgtgcagcctctgggttcacctttagcgactttgccatgagttgggtccgccaggctccagggaagggactggagtgggtctcagctatcggagctggtggttctaacacatacaacgcagactccgtgaagggccggttcaccatctccagggacaattccgcgaacacgctgtatttgcaaatgaatagcctgagagccgaggacacggccctatattactgtgcgaaacagaaacgagaatgcagtggctggtgcaaaggatggggtttggacgtctggggccaagggaccacggtcaccgtctcctca

SEQ ID NO: 60 - DNA, 337 bp, Homo sapiens; identical to SEQ ID NO: 52
  source 1..337: mol_type = other DNA

SEQ ID NO: 61 - DNA, 349 bp, Homo sapiens
  source 1..349: mol_type = other DNA
caggtgcagctacaacagtggggcgcaggactgttgaagccatcggagaccctgtccctcacctgcgctgtctatggtgggacgttcagtgattacttctatacctggatccgccagctcccagggaaggggctggagtggattggggaagtcagtcatactgggaggaccagctacatcccgtccctcaagagtcgagtcaccatgtcagtagacacgtctaaaaaccagttctccctgaacttgagctctgtgaccgacgcggacacggctgtttattattgtgggagaggtcggggttacacttggaatgattggggccagggaacgctagtcaccgtctcctcag

SEQ ID NO: 62 - DNA, 322 bp, Homo sapiens
  source 1..322: mol_type = other DNA
gacatccagatgacccagtctccatcctccctgtctgcctctgtaggagacagagtcaccatcacttgtcgggcaagtcagagcattagaacctctttaaattggtatcaacaaagaccagggagagcccctaaactcctgatctatgctgcaaccagtttgcaaggtgggttcccgtcaaggttcagtggcagtggatctgggacagatttcactctcaccatcagcagtctgcaacccgaagattttgcaacttactactgtcagcagagctacagtggccctccaacttttggccaggggaccaagttggatttcaaac

SEQ ID NO: 63 - DNA, 381 bp, Homo sapiens
  source 1..381: mol_type = other DNA
gaggtgcagctggtggagtctgggggaggcctggtcaagcctggggggtccctgagactctcctgtgcagcctctggattcagtttcagtagctatagcatgaactgggtccgccaggctccagggaaggggctggagtgggtctcatccattagtactagtggtaattacatatactacgcagactcagtgaagggccgattcaccatctccagagacaacgccaagaactcactgtatctgcaaatgaacagcctgagagccgaggacacggctgtatattactgtgcgagagatggacctccgatagtagcagctggcctgatctactactactacatgggcgtctggggcaaagggaccacggtcaccgtctcctca

SEQ ID NO: 64 - DNA, 319 bp, Homo sapiens
  source 1..319: mol_type = other DNA
gacatccagttgacccagtctccatcctccctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagatttcactctcaccatcagcagcctgcagcctgaagattttgcaacttattactgtcaacaccttaatagttaccggacttttggccaggggaccaagctggagatcaaac

SEQ ID NO: 65 - DNA, 367 bp, Homo sapiens
  source 1..367: mol_type = other DNA
caggtgcagcttcaggagtcgggcccaggactggtgaatccttcacaggtcctgtccctcacctgcgctgtctctggtgactccatcgtgactggtaagtactactggagctggatccgccaacaaccaggcaagggcttggagtggattggattcattgatcacagtggaactaccttctacaacccgtccctcaagagtcgcctctccatgtcagtagacacgtctaagaaccacttctccctccatctaaagtctgtgaccgccgcggacacggccgtctattattgtgcgagagaccagaagtatgagggagataattggttcgacccctggggccagggaaccctggtaatcgtctcctcag

SEQ ID NO: 66 - DNA, 331 bp, Homo sapiens
  source 1..331: mol_type = other DNA
cagtctgtgctgactcagccaccctcagcctctgggacccccgggcagagggtcaccatctcttgctctggaagcagctccaacgtcgcatataatagtgttaactggtaccagcaagtcccaggaacggcccccaaactcctcctctacgggactaatcggcggccctcaggggtccctgaccggttttctggctccaagtctggcacctcagcctccctggccatcagtgggctccagtctgaggatgaggctgattattattgttcggcgtgggatgagagcctgagtggtccggtcttcggcggggggaccaaactgaccgtcctcg

SEQ ID NO: 67 - DNA, 367 bp, Homo sapiens
  source 1..367: mol_type = other DNA
caggtgcagctggtgcagtctggggctgaggtgaagaagcctggggcctcagtgaaggtttcctgcaaggcatctggatacaccttcaccagatactatatgcactgggtgcgacaggcccctggacaagggcctgagtggatgggaataatcaaccctagtgatggtcgcacaggctacgcacagaagttccagggcagaatcacgctgaccagggacacgtccacgagcacagtctacatggagctgagcagcctgaaatctgaggacacggccatatattactgtgcgaccctctattactatgatgatagtggttattacactgactactggggccagggaaccctggtcaccgtctcctcag

SEQ ID NO: 68 - DNA, 325 bp, Homo sapiens
  source 1..325: mol_type = other DNA
gaaattgtgttgacgcagtctccaggcaccctgtctttgtctccaggggaaagagccaccctctcctgcagggccagtcagagtgtcagcaacaactacttaggctggttccagcagaaacctggccaggctcccaggctcctcatctatggtgcatcaagaagggccacgggcatcccagacaggttcactggtagtgggtctgggacagacttcactctcaccatcagcagactggagcctgaagattttgcagtgtattactgtcagcagtatggtaactcaccgtggacgttcggccaagggaccaaggtggaaatcaaac

SEQ ID NO: 69 - DNA, 367 bp, Homo sapiens
  source 1..367: mol_type = other DNA
cagatgcagctggtgcagtctggggctgaggtgaagaagactgggtcctcagtgaaggtttcctgcaaggcttccggatacaccttcacctaccgctacctgcactgggtgcgacaggcccccggacaagcgcttgagtggatgggttggatcacacctttcaatgataacaccaactacgcacagaaattccagggcagagtcaccattaccagggacaggtctatgagcacagcctacatggagttgagcagcctgagatctgaggacacagccatgtattactgtgctgtggcttatcactatgatagtagtggtccgtactttgaccactggggccagggaaccctggtcaccgtctcctcaa

SEQ ID NO: 70 - DNA, 337 bp, Homo sapiens
  source 1..337: mol_type = other DNA
cagtctgtgctgacgcagccgccctcagtgtctggggccccagggcagagggtcatcatctcctgcactgggagcagctccaacatcggggcaggttatgatgtacactggtaccagcagcttccaggaacagcccccaaactcctcgtctctagtagcagccatcggccctcaggggtccctgaccgattctctgcctccaagtctggcacctctgcctccctggccatcactgggctccaggctgaggatgaggctgattatttctgccagtcgtatgacagcagtctgagtggtcccgtggtattcggcggagggaccaagctgaccgtcctaa

SEQ ID NO: 71 - protein, 119 aa, Homo sapiens
QVQLVQSGAEVKKPGSSLKVSCKASGGILSSHVISWVRQAPGQGLEWMGGVIPVFGSTTYAPKFQDRLTVTADESTNTAYMDLSSLSSEDTAIYYCASYPYGKYYFDYWGQGTLVTVSS

SEQ ID NO: 72 - protein, 110 aa, Homo sapiens
QSVLTQPPSVSAAPGQKVTISCSGNSSNIGSNYVSWYQQVPGTAPKLLIYDNFKRPSDIPDRFSGSKSGTSATLGITGLQPGDEADYYCGAWDLGLNAGVFGGGTKLTVL

SEQ ID NO: 73 - protein, 125 aa, Homo sapiens
QVQLQQWGAGLLKPSETLSLTCAVYGGSPRGYYWSWIRQPPGKGLEWIGEINHSGTTNYNPSLTSRVTISADTSKSQFSLRLTSVTAADTAIYFCASSGESSRRGYFYYYGMDVWGQGTTVTVSS

SEQ ID NO: 74 - protein, 111 aa, Homo sapiens
QSVLTQPPSASGTPGQTVTISCSGISSNNENYYVYWYQQLPGAAPRLLIYRNSQRPSGVPDRFSGSKSDTSASLAISGLRSEDEADYYCAAWDDILSGPRVFGGGTKLTVL

SEQ ID NO: 75 - protein, 124 aa, Homo sapiens
QVQLVQSGAEVKKPGSSVKVSCKASEGTFSSCAISWVRQAPGQGLEWMGGIIPMFATTRYAQKFQDRVTITTDESTNTAYMELSSLRSEDTAIYYCARDGGGSFCGGDCYFDLWGQGTLVTVSS

SEQ ID NO: 76 - protein, 107 aa, Homo sapiens
EIVMTQSPATLSVSPGERATLSCRASQSVNSNLAWYQQKPGQAPRLLIFGASIRATGIPARFSGSGSGTEFTLTISSLQSEDFAVYYCQQYNNWPPTFGPGTKVDIK

SEQ ID NO: 77 - protein, 116 aa, Homo sapiens
QVQLVQSGVDVKKPGASVKLSCKTSGYTFTNYGITWVRQAPGQGLEWMGWISTYDGATNYSQNLQGRIIMTTDTSKRTAYLQMRSLRSDDTAVYYCARGRDSPDHWGQGTLVTVSS

SEQ ID NO: 78 - protein, 107 aa, Homo sapiens
DIQMTQSPSTLSASVGDRVTITCRASQSINRWLAWYQQKPGTAPKLLIFKASTLDSGVPARFSGTGSETEFSLTINSLQPDDFATYYCQQYDHFPHTFGPGTKLDIK

SEQ ID NO: 79 - protein, 120 aa, Homo sapiens
QVQLVESGGDLVTPGGSLRLSCAASGFAFSGYYMSWIRQAPGKGLEWISYINSNGLTISYADSVKGRFTVSRDNAKNSLFLQMSSLRAEDTAIYYCARDWGTTLVTFDLWGQGTLVTVSS

SEQ ID NO: 80 - protein, 106 aa, Homo sapiens
QPVLTQSSSASASMGSSAKLTCTLSSGHRGYNIAWLQQHPGKAPLYLTNLEGSDSYKNDRLTVSSSGADRYLTISNLQPEDEATYYCFTWDSDSRVFGGGTHLTVL

SEQ ID NO: 81 - protein, 119 aa, Homo sapiens
QVQLVESGGDLVKPGGSLRISCAASGFSFSDYYMSWIRQAPGKGLEWVAYISGSSAYTSYADSVKGRFSISRDNANNSLFLQMNSLRAEDTATYFCAKDYCGSGACYTADPGFFHQWAR

SEQ ID NO: 82 - protein, 110 aa, Homo sapiens
SYVLTQPPSVSVAPGKTATISCGGNNIGSKSVHWYQQKPGQAPIVVIYYDSDRPSGIPERFSGINSGNTATLTISRVEAGDEADYYCQVWDNTNDHPSYVFGAGTKVTXL

SEQ ID NO: 83 - protein, 121 aa, Homo sapiens
EVQLVQSGGDLVKPGGSLRLSCAASGFSFSNYRMNWVRQAPGKGLEWVSSITGSSSYIYYADSVQGRFTISRDNAKNSMSLQMKSLRVEDTAVYYCARDTYGDTDDAFDIWGQGTMVTVSS

SEQ ID NO: 84 - protein, 109 aa, Homo sapiens
SLELIQPPSVAVSPGQTARISCSGNALAKEYANWYQQRPGQAPVLVIFKDIERPTGIPERFSGSSSGTTLTLTISGVQAEDEANYYCHVVDRTETYRSVFGGGTKLTVL

SEQ ID NO: 85 - protein, 114 aa, Homo sapiens
EVQLVESGGGVVQPGRSLRLSCAGSGFTFSTYVMHWVRQAPGKGLEWVAVISYDGTNKYYADSMKGRFTISRDNSKNTLYLQLNRLRAEDTAVYYCAKAMDDSSGYYCPDYWAX

SEQ ID NO: 86 - protein, 109 aa, Homo sapiens
SSELTQDPAVSVALGQTVKITCQGDSLRNYFANWYQQKPGQAPVLVIYGQNNRPSGIPDRFSGSTSGNTGSLTITGAQAEDEADYYCNSRDSSGNHLYVFGTGTKVTVL

SEQ ID NO: 87 - protein, 123 aa, Homo sapiens
QVQLVQSGGGVVQPGRSLRLSCNASGFTFSKYGMHWVRQAPGKGLEWVAVITHDGGDKHHADSVKGRFSISRDNSKDILYLQMDSLRTEDTAIYYCAKDGPYAYRWGGFLDDWGQGTPVTVSS

SEQ ID NO: 88 - protein, 108 aa, Homo sapiens
EIVMTQSPVTLSVSPGERVTLSCRASESIGSSLAWYQQKPGQAPRLLIYGASTRTTGIPARFSGSGSGTEFTLAISSLQSEDYAVYFCQQYHSWPPLTFGGGTKVEIK

SEQ ID NO: 89 - protein, 125 aa, Homo sapiens
EVQLSESGGGLVQPGGSLRLSCAASGFTFSDFAMSWVRQAPGKGLEWVSAIGAGGSNTYNADSVKGRFTISRDNSANTLYLQMNSLRAEDTALYYCAKQKRECSGWCKGWGLDVWGQGTTVTVSS

SEQ ID NO: 90 - protein, 112 aa, Homo sapiens
DIVMTQSPLSLPVTPGEPASISCKSSQSLLDSKGSTYLDWYLQKPGQSPQLLIYLASYRASGVPDRFSGSGSGTDFTLKISRVEAEDVGVYYCMQALQTPYTFGQGTRLEIK

SEQ ID NO: 91 - protein, 122 aa, Homo sapiens
EVQLVESGGGLVKPGGSLRLSCAASGFTFSSSMMTWVRQAPGKGLEWVSSIGTGSNYKHYADSLRGRFTVSRDNAKNSLFLQMNSLRPEDTAVYYCARVMVVGLYYYYMDIWGTGTTVTVSS

SEQ ID NO: 92 - protein, 109 aa, Homo sapiens
EIVLTQSPGTLSLSPGERAALSCRASQNVSSNFLAWYQQNPGQAPRLLFYGASMRATGIPDRFSGSGSGTDFTLTISRLEPEDFAVYYCQLYGTSPTWTFGQGTKVEIK

SEQ ID NO: 93 - protein, 116 aa, Homo sapiens
EVQLVQSGPVLKKPGESLKISCRGSGYRFNTYWVAWVRQMPGKGLEWMGMIYPGDSDTKYSPSFQGQVTISADKSSNTAYLQWSSLKASDTAMYYCAREVYVASTDSDYYGMDVAD

SEQ ID NO: 94 - protein, 110 aa, Homo sapiens
SLXLTQPASVSGSPGQSITISCTGTSNDVGRSDLVAWYQQHPDKAPRLIIYESSKRPSGVSARFSGSXSGITASLTISDLQAEDEADYYCCSYAGGNTYVFGTATGVTVL

SEQ ID NO: 95 - protein, 111 aa, Homo sapiens
EVQLVETGGGLIQPGGSLRLSCAASGFSVSTRFMSWVRQAPGQGLEWVSVVYKDGDTFNSDSVKGRFSISRDNSKNTVFLQMNRLRVEDTAVYFCVRHGDGWNYVDSWGLE

SEQ ID NO: 96 - protein, 107 aa, Homo sapiens
EIVMTQSPATLSVSPGEGATLSCRASHSLSSHLAWYQQKPGQAPRLLIYDASVRATDIPARFSGSGSGTEFTLTISSLQSEDFAVYYCQQYNNWPLTFGGGTKXRSX

SEQ ID NO: 97 - protein, 125 aa, Homo sapiens
EVQLSESGGGLVRPGGSLRLSCAASGFTFSDFAMSWVRQAPGKGLEWVSAIGAGGSNTYNADSVKGRFTISRDNSANTLYLQMNSLRAEDTALYYCAKQKRECSGWCKGWGLDVWGQGTTVTVSS

SEQ ID NO: 98 - protein, 112 aa, Homo sapiens; identical to SEQ ID NO: 90

SEQ ID NO: 99 - protein, 116 aa, Homo sapiens
QVQLQQWGAGLLKPSETLSLTCAVYGGTFSDYFYTWIRQLPGKGLEWIGEVSHTGRTSYIPSLKSRVTMSVDTSKNQFSLNLSSVTDADTAVYYCGRGRGYTWNDWGQGTLVTVSS

SEQ ID NO: 100 - protein, 107 aa, Homo sapiens
DIQMTQSPSSLSASVGDRVTITCRASQSIRTSLNWYQQRPGRAPKLLIYAATSLQGGFPSRFSGSGSGTDFTLTISSLQPEDFATYYCQQSYSGPPTFGQGTKLDFK

SEQ ID NO: 101 - protein, 127 aa, Homo sapiens
EVQLVESGGGLVKPGGSLRLSCAASGFSFSSYSMNWVRQAPGKGLEWVSSISTSGNYIYYADSVKGRFTISRDNAKNSLYLQMNSLRAEDTAVYYCARDGPPIVAAGLIYYYYMGVWGKGTTVTVSS

SEQ ID NO: 102 - protein, 106 aa, Homo sapiens
DIQLTQSPSSLSASVGDRVTITCRASQGISSYLAWYQQKPGKAPKLLIYAASTLQSGVPSRFSGSGSGTDFTLTISSLQPEDFATYYCQHLNSYRTFGQGTKLEIK

SEQ ID NO: 103 - protein, 122 aa, Homo sapiens
QVQLQESGPGLVNPSQVLSLTCAVSGDSIVTGKYYWSWIRQQPGKGLEWIGFIDHSGTTFYNPSLKSRLSMSVDTSKNHFSLHLKSVTAADTAVYYCARDQKYEGDNWFDPWGQGTLVIVSS

SEQ ID NO: 104 - protein, 110 aa, Homo sapiens
QSVLTQPPSASGTPGQRVTISCSGSSSNVAYNSVNWYQQVPGTAPKLLLYGTNRRPSGVPDRFSGSKSGTSASLAISGLQSEDEADYYCSAWDESLSGPVFGGGTKLTVL

SEQ ID NO: 105 - protein, 122 aa, Homo sapiens
QVQLVQSGAEVKKPGASVKVSCKASGYTFTRYYMHWVRQAPGQGPEWMGIINPSDGRTGYAQKFQGRITLTRDTSTSTVYMELSSLKSEDTAIYYCATLYYYDDSGYYTDYWGQGTLVTVSS

SEQ ID NO: 106 - protein, 108 aa, Homo sapiens
EIVLTQSPGTLSLSPGERATLSCRASQSVSNNYLGWFQQKPGQAPRLLIYGASRRATGIPDRFTGSGSGTDFTLTISRLEPEDFAVYYCQQYGNSPWTFGQGTKVEIK

SEQ ID NO: 107 - protein, 122 aa, Homo sapiens
QMQLVQSGAEVKKTGSSVKVSCKASGYTFTYRYLHWVRQAPGQALEWMGWITPFNDNTNYAQKFQGRVTITRDRSMSTAYMELSSLRSEDTAMYYCAVAYHYDSSGPYFDHWGQGTLVTVSS

SEQ ID NO: 108 - protein, 112 aa, Homo sapiens
QSVLTQPPSVSGAPGQRVIISCTGSSSNIGAGYDVHWYQQLPGTAPKLLVSSSSHRPSGVPDRFSASKSGTSASLAITGLQAEDEADYFCQSYDSSLSGPVVFGGGTKLTVL

SEQ ID NO: 109 - DNA, 24 bp, Homo sapiens
  source 1..24: mol_type = other DNA
ggaggcatcctcagtagtcatgtt

SEQ ID NO: 110 - DNA, 24 bp, Homo sapiens
  source 1..24: mol_type = other DNA
gtcatccctgtctttggttcaaca

SEQ ID NO: 111 - DNA, 36 bp, Homo sapiens
  source 1..36: mol_type = other DNA
gcgtcttacccctatggcaaatactactttgactac

SEQ ID NO: 112 - protein, 8 aa, Homo sapiens
GGILSSHV

SEQ ID NO: 113 - protein, 8 aa, Homo sapiens
VIPVFGST

SEQ ID NO: 114 - protein, 12 aa, Homo sapiens
ASYPYGKYYFDY

SEQ ID NO: 115 - DNA, 24 bp, Homo sapiens
  source 1..24: mol_type = other DNA
ggtgggtccccaaggggatactac

SEQ ID NO: 116 - DNA, 21 bp, Homo sapiens
  source 1..21: mol_type = other DNA
atcaatcacagtggaaccacc

SEQ ID NO: 117 - DNA, 57 bp, Homo sapiens
  source 1..57: mol_type = other DNA
gcgagcagcggggaaagcagcagacggggctacttctactattacggtatggacgtc

SEQ ID NO: 118 - protein, 8 aa, Homo sapiens
GGSPRGYY

SEQ ID NO: 119 - protein, 7 aa, Homo sapiens
INHSGTT

SEQ ID NO: 120 - protein, 19 aa, Homo sapiens
ASSGESSRRGYFYYYGMDV

SEQ ID NO: 121 - DNA, 24 bp, Homo sapiens
  source 1..24: mol_type = other DNA
gaaggcaccttcagcagctgtgct

SEQ ID NO: 122 - DNA, 24 bp, Homo sapiens
  source 1..24: mol_type = other DNA
atcatccctatgtttgctacaaca

SEQ ID NO: 123 - DNA, 51 bp, Homo sapiens
  source 1..51: mol_type = other DNA
gcgagagacggcggggggagcttttgtggtggtgattgttatttcgactta

SEQ ID NO: 124 - protein, 8 aa, Homo sapiens
EGTFSSCA

SEQ ID NO: 125 - protein, 8 aa, Homo sapiens
IIPMFATT

SEQ ID NO: 126 - protein, 17 aa, Homo sapiens
ARDGGGSFCGGDCYFDL

SEQ ID NO: 127 - DNA, 24 bp, Homo sapiens
  source 1..24: mol_type = other DNA
ggttacacctttactaattatggt

SEQ ID NO: 128 - DNA, 24 bp, Homo sapiens
  source 1..24: mol_type = other DNA
atcagcacttacgatggtgccaca

SEQ ID NO: 129 - DNA, 27 bp, Homo sapiens
  source 1..27: mol_type = other DNA
gcgaggggacgagatagtccggaccac

SEQ ID NO: 130 - protein, 8 aa, Homo sapiens
GYTFTNYG

SEQ ID NO: 131 - protein, 8 aa, Homo sapiens
ISTYDGAT

SEQ ID NO: 132 - protein, 9 aa, Homo sapiens
ARGRDSPDH

SEQ ID NO: 133 - DNA, 24 bp, Homo sapiens
  source 1..24: mol_type = other DNA
ggattcgcctttagcggctactac

SEQ ID NO: 134 - DNA, 24 bp, Homo sapiens
  source 1..24: mol_type = other DNA
attaatagtaacggtcttaccatc

SEQ ID NO: 135 - DNA, 39 bp, Homo sapiens
  source 1..39: mol_type = other DNA
gcgcgagattgggggacaacattggtaacttttgacctc

SEQ ID NO: 136 - protein, 8 aa, Homo sapiens
GFAFSGYY

SEQ ID NO: 137 - protein, 8 aa, Homo sapiens
INSNGLTI

SEQ ID NO: 138 - protein, 13 aa, Homo sapiens
ARDWGTTLVTFDL

SEQ ID NO: 139 - DNA, 24 bp, Homo sapiens
  source 1..24: mol_type = other DNA
ggattcagcttcagtgactactac

SEQ ID NO: 140 - DNA, 24 bp, Homo sapiens
  source 1..24: mol_type = other DNA
attagtggatccagtgcctacaca

SEQ ID NO: 141 - DNA, 60 bp, Homo sapiens
  source 1..60: mol_type = other DNA
gcgaaagattactgtggcagtggcgcctgctacactgcggaccctggcttcttccatcaa

SEQ ID NO: 142 - protein, 8 aa, Homo sapiens
GFSFSDYY

SEQ ID NO: 143 - protein, 8 aa, Homo sapiens
ISGSSAYT

SEQ ID NO: 144 - protein, 20 aa, Homo sapiens
AKDYCGSGACYTADPGFFHQ

SEQ ID NO: 145 - DNA, 24 bp, Homo sapiens
  source 1..24: mol_type = other DNA
ggattcagtttcagtaattatcga

SEQ ID NO: 146 - DNA, 24 bp, Homo sapiens
  source 1..24: mol_type = other DNA
attactggcagtagtagttacatc

SEQ ID NO: 147 - DNA, 42 bp, Homo sapiens
  source 1..42: mol_type = other DNA
gcgagagatacgtacggtgacaccgatgatgcttttgatatt

SEQ ID NO: 148 - protein, 8 aa, Homo sapiens
GFSFSNYR

SEQ ID NO: 149 - protein, 8 aa, Homo sapiens
ITGSSSYI

SEQ ID NO: 150 - protein, 14 aa, Homo sapiens
ARDTYGDTDDAFDI

SEQ ID NO: 151 - DNA, 24 bp, Homo sapiens
  source 1..24: mol_type = other DNA
ggattcaccttcagtacctatgtc

SEQ ID NO: 152 - DNA, 24 bp, Homo sapiens
  source 1..24: mol_type = other DNA
atatcatatgatggaactaataaa

SEQ ID NO: 153 - DNA, 45 bp, Homo sapiens
  source 1..45: mol_type = other DNA
gcgaaagcaatggatgatagtagtggttattattgtcctgattac

SEQ ID NO: 154 - protein, 8 aa, Homo sapiens
GFTFSTYV

SEQ ID NO: 155 - protein, 8 aa, Homo sapiens
ISYDGTNK

SEQ ID NO: 156 - protein, 15 aa, Homo sapiens
AKAMDDSSGYYCPDY

SEQ ID NO: 157 - DNA, 24 bp, Homo sapiens
  source 1..24: mol_type = other DNA
ggattcaccttcagtaaatatggc

SEQ ID NO: 158 - DNA, 24 bp, Homo sapiens
  source 1..24: mol_type = other DNA
atcacacatgatggaggtgataaa

SEQ ID NO: 159 - DNA, 48 bp, Homo sapiens
  source 1..48: mol_type = other DNA
gcgaaagacggcccctatgcttaccgttgggggggtttcttggacgac

SEQ ID NO: 160 - protein, 8 aa, Homo sapiens
GFTFSKYG

SEQ ID NO: 161 - protein, 8 aa, Homo sapiens
ITHDGGDK

SEQ ID NO: 162 - protein, 16 aa, Homo sapiens
AKDGPYAYRWGGFLDD

SEQ ID NO: 163 - DNA, 24 bp, Homo sapiens
  source 1..24: mol_type = other DNA
gggttcacctttagcgactttgcc

SEQ ID NO: 164 - DNA, 24 bp, Homo sapiens
  source 1..24: mol_type = other DNA
atcggagctggtggttctaacaca

SEQ ID NO: 165 - DNA, 54 bp, Homo sapiens
  source 1..54: mol_type = other DNA
gcgaaacagaaacgagaatgcagtggctggtgcaaaggatggggtttggacgtc

SEQ ID NO: 166 - protein, 8 aa, Homo sapiens
GFTFSDFA

SEQ ID NO: 167 - protein, 8 aa, Homo sapiens
IGAGGSNT

SEQ ID NO: 168 - protein, 18 aa, Homo sapiens
AKQKRECSGWCKGWGLDV

SEQ ID NO: 169 - DNA, 24 bp, Homo sapiens
  source 1..24: mol_type = other DNA
ggattcaccttcagttcgtctatg

SEQ ID NO: 170 - DNA, 24 bp, Homo sapiens
  source 1..24: mol_type = other DNA
attggaactggtagtaactacaaa

SEQ ID NO: 171 - DNA, 45 bp, Homo sapiens
  source 1..45: mol_type = other DNA
gcgagagtaatggtagtagggctttactactactacatggacatc

SEQ ID NO: 172 - protein, 8 aa, Homo sapiens
GFTFSSSM

SEQ ID NO: 173 - protein, 8 aa, Homo sapiens
IGTGSNYK

SEQ ID NO: 174 - protein, 15 aa, Homo sapiens
ARVMVVGLYYYYMDI

SEQ ID NO: 175 - DNA, 24 bp, Homo sapiens
  source 1..24: mol_type = other DNA
ggatacaggttcaacacttattgg

SEQ ID NO: 176 - DNA, 24 bp, Homo sapiens
  source 1..24: mol_type = other DNA
atctatccgggtgactcggatacg

SEQ ID NO: 177 - DNA, 54 bp, Homo sapiens
  source 1..54: mol_type = other DNA
gcgagagaagtatatgtggcttcgactgatagtgactattacggtatggacgtc

SEQ ID NO: 178 - protein, 8 aa, Homo sapiens
GYRFNTYW

SEQ ID NO: 179 - protein, 8 aa, Homo sapiens
IYPGDSDT

SEQ ID NO: 180 - protein, 18 aa, Homo sapiens
AREVYVASTDSDYYGMDV

SEQ ID NO: 181 - DNA, 24 bp, Homo sapiens
  source 1..24: mol_type = other DNA
gggttcagcgtcagtacgaggttc

SEQ ID NO: 182 - DNA, 21 bp, Homo sapiens
  source 1..21: mol_type = other DNA
gtctataaagatggtgacacc

SEQ ID NO: 183 - DNA, 36 bp, Homo sapiens
  source 1..36: mol_type = other DNA
gtgcgacatggcgatggttggaattacgtcgactcc

SEQ ID NO: 184 - protein, 8 aa, Homo sapiens
GFSVSTRF

SEQ ID NO: 185 - protein, 7 aa, Homo sapiens
VYKDGDT

SEQ ID NO: 186 - protein, 12 aa, Homo sapiens
VRHGDGWNYVDS

SEQ ID NO: 187 - DNA, 24 bp, Homo sapiens
  source 1..24: mol_type = other DNA
gggttcacctttagcgactttgcc

SEQ ID NO: 188 - DNA, 24 bp, Homo sapiens
  source 1..24: mol_type = other DNA
atcggagctggtggttctaacaca

SEQ ID NO: 189 - DNA, 54 bp, Homo sapiens; identical to SEQ ID NO: 165
  source 1..54: mol_type = other DNA

SEQ ID NO: 190 - protein, 8 aa, Homo sapiens
GFTFSDFA

SEQ ID NO: 191 - protein, 8 aa, Homo sapiens
IGAGGSNT

SEQ ID NO: 192 - protein, 18 aa, Homo sapiens
AKQKRECSGWCKGWGLDV

SEQ ID NO: 193 - DNA, 24 bp, Homo sapiens
  source 1..24: mol_type = other DNA
ggtgggacgttcagtgattacttc

SEQ ID NO: 194 - DNA, 21 bp, Homo sapiens
  source 1..21: mol_type = other DNA
gtcagtcatactgggaggacc

SEQ ID NO: 195 - DNA, 30 bp, Homo sapiens
  source 1..30: mol_type = other DNA
gggagaggtcggggttacacttggaatgat

SEQ ID NO: 196 - protein, 8 aa, Homo sapiens
GGTFSDYF

SEQ ID NO: 197 - protein, 7 aa, Homo sapiens
VSHTGRT

SEQ ID NO: 198 - protein, 10 aa, Homo sapiens
GRGRGYTWND

SEQ ID NO: 199 - DNA, 24 bp, Homo sapiens
  source 1..24: mol_type = other DNA
ggattcagtttcagtagctatagc

SEQ ID NO: 200 - DNA, 24 bp, Homo sapiens
  source 1..24: mol_type = other DNA
attagtactagtggtaattacata

SEQ ID NO: 201 - DNA, 60 bp, Homo sapiens
  source 1..60: mol_type = other DNA
gcgagagatggacctccgatagtagcagctggcctgatctactactactacatgggcgtc

SEQ ID NO: 202 - protein, 8 aa, Homo sapiens
GFSFSSYS

SEQ ID NO: 203 - protein, 8 aa, Homo sapiens
ISTSGNYI

SEQ ID NO: 204 - protein, 20 aa, Homo sapiens
ARDGPPIVAAGLIYYYYMGV

SEQ ID NO: 205 - DNA, 30 bp, Homo sapiens
  source 1..30: mol_type = other DNA
ggtgactccatcgtgactggtaagtactac

SEQ ID NO: 206 - DNA, 21 bp, Homo sapiens
  source 1..21: mol_type = other DNA
attgatcacagtggaactacc

SEQ ID NO: 207 - DNA, 42 bp, Homo sapiens
  source 1..42: mol_type = other DNA
gcgagagaccagaagtatgagggagataattggttcgacccc

SEQ ID NO: 208 - protein, 10 aa, Homo sapiens
GDSIVTGKYY

SEQ ID NO: 209 - protein, 7 aa, Homo sapiens
IDHSGTT

SEQ ID NO: 210 - protein, 14 aa, Homo sapiens
ARDQKYEGDNWFDP

SEQ ID NO: 211 - DNA, 24 bp, Homo sapiens
  source 1..24: mol_type = other DNA
ggatacaccttcaccagatactat

SEQ ID NO: 212 - DNA, 24 bp, Homo sapiens
  source 1..24: mol_type = other DNA
atcaaccctagtgatggtcgcaca

SEQ ID NO: 213 - DNA, 45 bp, Homo sapiens
  source 1..45: mol_type = other DNA
gcgaccctctattactatgatgatagtggttattacactgactac

SEQ ID NO: 214 - protein, 8 aa, Homo sapiens
GYTFTRYY

SEQ ID NO: 215 - protein, 8 aa, Homo sapiens
INPSDGRT

SEQ ID NO: 216 - protein, 15 aa, Homo sapiens
ATLYYYDDSGYYTDY

SEQ ID NO: 217 - DNA, 24 bp, Homo sapiens
  source 1..24: mol_type = other DNA
ggatacaccttcacctaccgctac

SEQ ID NO: 218 - DNA, 24 bp, Homo sapiens
  source 1..24: mol_type = other DNA
atcacacctttcaatgataacacc

SEQ ID NO: 219 - DNA, 45 bp, Homo sapiens
  source 1..45: mol_type = other DNA
gctgtggcttatcactatgatagtagtggtccgtactttgaccac

SEQ ID NO: 220 - protein, 8 aa, Homo sapiens
GYTFTYRY

SEQ ID NO: 221 - protein, 8 aa, Homo sapiens
ITPFNDNT

SEQ ID NO: 222 - protein, 15 aa, Homo sapiens
AVAYHYDSSGPYFDH

SEQ ID NO: 223 - DNA, 24 bp, Homo sapiens
  source 1..24: mol_type = other DNA
agctccaacattggcagtaattat

SEQ ID NO: 224 - skipped

SEQ ID NO: 225 - DNA, 33 bp, Homo sapiens
  source 1..33: mol_type = other DNA
ggagcatgggatctcggcctcaacgctggggtg

SEQ ID NO: 226 - protein, 8 aa, Homo sapiens
SSNIGSNY

SEQ ID NO: 227 - skipped

SEQ ID NO: 228 - protein, 11 aa, Homo sapiens
GAWDLGLNAGV

SEQ ID NO: 229 - DNA, 24 bp, Homo sapiens
  source 1..24: mol_type = other DNA
agctccaacaacgaaaattattat

SEQ ID NO: 230 - skipped

SEQ ID NO: 231 - DNA, 36 bp, Homo sapiens
  source 1..36: mol_type = other DNA
gcagcgtgggatgacatcctgagtggtccgagggtg

SEQ ID NO: 232 - protein, 8 aa, Homo sapiens
SSNNENYY

SEQ ID NO: 233 - skipped

SEQ ID NO: 234 - protein, 12 aa, Homo sapiens
AAWDDILSGPRV

SEQ ID NO: 235 - DNA, 18 bp, Homo sapiens
  source 1..18: mol_type = other DNA
cagagtgttaacagcaac

SEQ ID NO: 236 - skipped

SEQ ID NO: 237 - DNA, 27 bp, Homo sapiens
  source 1..27: mol_type = other DNA
cagcagtataataactggcctcccact

SEQ ID NO: 238 - protein, 6 aa, Homo sapiens
QSVNSN

SEQ ID NO: 239 - skipped

SEQ ID NO: 240 - protein, 9 aa, Homo sapiens
QQYNNWPPT

SEQ ID NO: 241 - DNA, 18 bp, Homo sapiens
  source 1..18: mol_type = other DNA
cagagtattaataggtgg

SEQ ID NO: 242 - skipped

SEQ ID NO: 243 - DNA, 27 bp, Homo sapiens
  source 1..27: mol_type = other DNA
caacagtatgatcattttccgcacact

SEQ ID NO: 244 - protein, 6 aa, Homo sapiens
QSINRW

SEQ ID NO: 245 - skipped

SEQ ID NO: 246 - protein, 9 aa, Homo sapiens
QQYDHFPHT

SEQ ID NO: 247 - DNA, 21 bp, Homo sapiens
  source 1..21: mol_type = other DNA
agtggccacagaggctacaac

SEQ ID NO: 248 - DNA, 21 bp, Homo sapiens
  source 1..21: mol_type = other DNA
cttgagggtagtgactcctac

SEQ ID NO: 249 - DNA, 27 bp, Homo sapiens
  source 1..27: mol_type = other DNA
ttcacctgggacagcgactcccgcgtc

SEQ ID NO: 250 - protein, 7 aa, Homo sapiens
SGHRGYN

SEQ ID NO: 251 - protein, 7 aa, Homo sapiens
LEGSDSY

SEQ ID NO: 252 - protein, 9 aa, Homo sapiens
FTWDSDSRV

SEQ ID NO: 253 - DNA, 18 bp, Homo sapiens
  source 1..18: mol_type = other DNA
aacattggaagtaaaagt

SEQ ID NO: 254 - skipped

SEQ ID NO: 255 - DNA, 39 bp, Homo sapiens
  source 1..39: mol_type = other DNA
caggtgtgggacaatactaatgatcatccctcttatgtc

SEQ ID NO: 256 - protein, 6 aa, Homo sapiens
NIGSKS

SEQ ID NO: 257 - skipped

SEQ ID NO: 258 - protein, 13 aa, Homo sapiens
QVWDNTNDHPSYV

SEQ ID NO: 259 - DNA, 18 bp, Homo sapiens
  source 1..18: mol_type = other DNA
gcattggcaaaggaatat

SEQ ID NO: 260 - skipped

SEQ ID NO: 261 - DNA, 36 bp, Homo sapiens
  source 1..36: mol_type = other DNA
cacgtagttgacaggactgagacgtatcgaagtgtc

SEQ ID NO: 262 - protein, 6 aa, Homo sapiens
ALAKEY

SEQ ID NO: 263 - skipped

SEQ ID NO: 264 - protein, 12 aa, Homo sapiens
HVVDRTETYRSV

SEQ ID NO: 265 - DNA, 18 bp, Homo sapiens
  source 1..18: mol_type = other DNA
agcctcagaaactatttt

SEQ ID NO: 266 - skipped

SEQ ID NO: 267 - DNA, 36 bp, Homo sapiens
  source 1..36: mol_type = other DNA
aactcccgggacagcagtggtaaccatctttatgtc

SEQ ID NO: 268 - protein, 6 aa, Homo sapiens
SLRNYF

SEQ ID NO: 269 - skipped

SEQ ID NO: 270 - protein, 12 aa, Homo sapiens
NSRDSSGNHLYV

SEQ ID NO: 271 - DNA, 18 bp, Homo sapiens
  source 1..18: mol_type = other DNA
gagagtattggcagcagt

SEQ ID NO: 272 - skipped

SEQ ID NO: 273 - DNA, 30 bp, Homo sapiens
  source 1..30: mol_type = other DNA
cagcagtatcatagctggcccccgctcact

SEQ ID NO: 274 - protein, 6 aa, Homo sapiens
ESIGSS

SEQ ID NO: 275 - skipped

SEQ ID NO: 276 - protein, 10 aa, Homo sapiens
QQYHSWPPLT

SEQ ID NO: 277 - DNA, 33 bp, Homo sapiens
  source 1..33: mol_type = other DNA
cagagcctcctggatagtaagggatccacctat

SEQ ID NO: 278 - skipped

SEQ ID NO: 279 - DNA, 27 bp, Homo sapiens
  source 1..27: mol_type = other DNA
atgcaagctctacaaactccttacact

SEQ ID NO: 280 - protein, 11 aa, Homo sapiens
QSLLDSKGSTY

SEQ ID NO: 281 - skipped

SEQ ID NO: 282 - protein, 9 aa, Homo sapiens
MQALQTPYT

SEQ ID NO: 283 - DNA, 21 bp, Homo sapiens
  source 1..21: mol_type = other DNA
cagaatgttagtagcaacttc

SEQ ID NO: 284 - skipped

SEQ ID NO: 285 - DNA, 30 bp, Homo sapiens
  source 1..30: mol_type = other DNA
caactgtacggtacctcaccaacgtggacg

SEQ ID NO: 286 - protein, 7 aa, Homo sapiens
QNVSSNF

SEQ ID NO: 287 - skipped

SEQ ID NO: 288 - protein, 10 aa, Homo sapiens
QLYGTSPTWT

SEQ ID NO: 289 - DNA, 27 bp, Homo sapiens
  source 1..27: mol_type = other DNA
agcaatgatgttgggcgttctgacctt

SEQ ID NO: 290 - skipped

SEQ ID NO: 291 - DNA, 30 bp, Homo sapiens
  source 1..30: mol_type = other DNA
tgttcatatgcaggtggtaacacttatgtc

SEQ ID NO: 292 - protein, 9 aa, Homo sapiens
SNDVGRSDL

SEQ ID NO: 293 - skipped

SEQ ID NO: 294 - protein, 10 aa, Homo sapiens
CSYAGGNTYV

SEQ ID NO: 295 - DNA, 18 bp, Homo sapiens
  source 1..18: mol_type = other DNA
cacagtcttagtagtcac

SEQ ID NO: 296 - skipped

SEQ ID NO: 297 - DNA, 27 bp, Homo sapiens
  source 1..27: mol_type = other DNA
cagcaatataataactggccgctcact

SEQ ID NO: 298 - protein, 6 aa, Homo sapiens
HSLSSH

SEQ ID NO: 299 - skipped

SEQ ID NO: 300 - protein, 9 aa, Homo sapiens
QQYNNWPLT

SEQ ID NO: 301 - DNA, 33 bp, Homo sapiens; identical to SEQ ID NO: 277
  source 1..33: mol_type = other DNA

SEQ ID NO: 302 - skipped

SEQ ID NO: 303 - DNA, 27 bp, Homo sapiens
  source 1..27: mol_type = other DNA
atgcaagctctacaaactccttacact

SEQ ID NO: 304 - protein, 11 aa, Homo sapiens
QSLLDSKGSTY

SEQ ID NO: 305 - skipped

SEQ ID NO: 306 - protein, 9 aa, Homo sapiens
MQALQTPYT

SEQ ID NO: 307 - DNA, 18 bp, Homo sapiens
  source 1..18: mol_type = other DNA
cagagcattagaacctct

SEQ ID NO: 308 - skipped

SEQ ID NO: 309 - DNA, 27 bp, Homo sapiens
  source 1..27: mol_type = other DNA
cagcagagctacagtggccctccaact

SEQ ID NO: 310 - protein, 6 aa, Homo sapiens
QSIRTS

SEQ ID NO: 311 - skipped

SEQ ID NO: 312 - protein, 9 aa, Homo sapiens
QQSYSGPPT

SEQ ID NO: 313 - DNA, 18 bp, Homo sapiens
  source 1..18: mol_type = other DNA
cagggcattagcagttat

SEQ ID NO: 314 - skipped

SEQ ID NO: 315 - DNA, 24 bp, Homo sapiens
  source 1..24: mol_type = other DNA
caacaccttaatagttaccggact

SEQ ID NO: 316 - protein, 6 aa, Homo sapiens
QGISSY

SEQ ID NO: 317 - skipped

SEQ ID NO: 318 - protein, 8 aa, Homo sapiens
QHLNSYRT

SEQ ID NO: 319 - DNA, 24 bp, Homo sapiens
  source 1..24: mol_type = other DNA
agctccaacgtcgcatataatagt

SEQ ID NO: 320 - skipped

SEQ ID NO: 321 - DNA, 33 bp, Homo sapiens
  source 1..33: mol_type = other DNA
tcggcgtgggatgagagcctgagtggtccggtc

SEQ ID NO: 322 - protein, 8 aa, Homo sapiens
SSNVAYNS

SEQ ID NO: 323 - skipped

SEQ ID NO: 324 - protein, 11 aa, Homo sapiens
SAWDESLSGPV

SEQ ID NO: 325 - DNA, 21 bp, Homo sapiens
  source 1..21: mol_type = other DNA
cagagtgtcagcaacaactac

SEQ ID NO: 326 - skipped

SEQ ID NO: 327 - DNA, 27 bp, Homo sapiens
  source 1..27: mol_type = other DNA
cagcagtatggtaactcaccgtggacg

SEQ ID NO: 328 - protein, 7 aa, Homo sapiens
QSVSNNY

SEQ ID NO: 329 - skipped

SEQ ID NO: 330 - protein, 9 aa, Homo sapiens
QQYGNSPWT

SEQ ID NO: 331 - DNA, 27 bp, Homo sapiens
  source 1..27: mol_type = other DNA
agctccaacatcggggcaggttatgat

SEQ ID NO: 332 - skipped

SEQ ID NO: 333 - DNA, 36 bp, Homo sapiens
  source 1..36: mol_type = other DNA
cagtcgtatgacagcagtctgagtggtcccgtggta

SEQ ID NO: 334 - protein, 9 aa, Homo sapiens
SSNIGAGYD

SEQ ID NO: 335 - skipped

SEQ ID NO: 336 - protein, 12 aa, Homo sapiens
QSYDSSLSGPVV

SEQ ID NO: 337 - protein, 137 aa, Homo sapiens
QQQQQQQEQLEVPPFLVGAPQSVIKQFYDLLKADETKTDAQTEADVEAFINRLGGTYKTRFDQFKQEIKQGKAAYERLHQQAVAKFSKEAREADAKMSAIADSPSLTTQQKTQQIQAIMDSLSEAVRKEILDALSQL

SEQ ID NO: 338 - protein, 166 aa, Homo sapiens
AQREAQIPQSDIPPFLSGAPNHVVKQFFDLLRADESKTDPQTEADIEAFMRRLGGVYQARFEQFKQEMKKQFAQYDKVHQAALSRFSPAARQADARMSAIAESKQLTVKQKTEQIKAIMDSLSESVRKEILEGFNSKFCVISVLLNVTIKIFSKWRKNHMRQKSNK

SEQ ID NO: 339 - protein, 137 aa, Homo sapiens
AQREAQLPQPEIPPFLSGVPSHVVKQFFDLLKADESKTDPQTEADIEAFIRRLGGDYQTRFEQFKQEIKKEKAQYEKIHQAALLKFSPAAREADAKMSAIADSTQLTNHQKTEQIKAIMDSLSEAVRKEILEGFNSQ

SEQ ID NO: 340 - protein, 137 aa, Homo sapiens
MLYAVVFLFSFAGFPSFLNAICTTSHVTVTGQLGCGERALKNVIVELREHDILDPDDSLNKTKSDNKGYFTIYGEECEVGSIEPYLRIIHNCEDGILSENCIITDDFPIPNDQIGKIYKLGIVSLNIARMNHKKHCH

SEQ ID NO: 341 - protein, 135 aa, Homo sapiens
MLTLKVTGFTLLLVTFAYCKMQTMTVRGQVACSDKSAINVHIELREADTWDPDDSLSATHSDRNGRFEVSGQEDEIGSIIEPYLRITHSCNDGVIDPKCRIMDDYQIPKEYINDIYNMGIVSLNIAQEGREKKCI

SEQ ID NO: 342 - protein, 139 aa, Homo sapiens
MRLTLTFIIISGISIPIITALKMKSVGVNGTVICDGKPVNDAWIELYSERNAGKSNAVLAKTKTNEQGHFMIKGSSKTDMFDPQFTISHKCRTKLCTRRVFLRIPDKYFTLSSEPYEMYDIGVVDMKKKFLTEIKTCPT

SEQ ID NO: 343 - protein, 154 aa, Homo sapiens
MILVHILCLFLHLVIGDQIKFAEKISTHSTAIKGILLCGNTSANDVKVKLFRKASDDIGEVMSTDQTTLNGHFQIKGDTVGRTEQDIEPVIRFYHRCDDDLKKDLKKVGYRTFAISYPKEYVTIGKVPRKQFDIGKLNLQIIYPRESRDMKFVD

SEQ ID NO: 344 - protein, 147 aa, Homo sapiens
MEWILFILLPTIANALFPLSRVQRVAVQGILLCEGRPLPNHQIILIDHDSSESIYLELDPDDIMAMNVTDQRGYFFVHGNETEWSTIDPVLVVRHKCNDGGIPCDREWRLGIPVKYISNEGDVEHIMDIGILNAEVVFYGEKRDCLL

SEQ ID NO: 345 - protein, 147 aa, Homo sapiens
MFHILLLCLFNFSGIFGVLGGLIGRTQSAGIEGTLMCDGKPLPGVLIKLYDDDRGIDTDDLMAKGKSDSKGHFRISGYTSEITTIDPKLNIYHDCNDNWVVIPCQRKISVMIPDKYVSSGKTPKQFYNAGNIELSGKFKGETRDCVH

SEQ ID NO: 346 - protein, 146 aa, Homo sapiens
MIRQILLLTTFLIPSIYGAAGGLVGRTQSAGAKGYLMCNGVPESGVLIKLYDDDRGLDFDDFMGETVTDSRGFFEITGRNAEVTPIDPKINIYHDCNDGWWPCQRKISIMIPDDYIAIGNAPTKLYDVGTIELAGKYNGETRDCIH

SEQ ID NO: 347 - protein, 144 aa, Homo sapiens
MQRKSTFLFLIFTIFHQQVIVISFRQQSVGIRGRLLCGNQSLPNTQIKLWNKNKIGTDDQLAATKTKADGSYELKGGIGSIFGMDVVLTIYHDCDDGIMPCKRKVALRIPSQYVTRSDDVRQWFNAGDLNMEFKFPDEGRSCIN

SEQ ID NO: 348 - protein, 138 aa, Homo sapiens
MFIFLALSITLCSVNAFRTQSAGVRGTLMCGNSPLKNTKVKLWDEDAIDMDDLLEEGRTNAQGHFELSGYTSEITTINPILKIYHDCNDRIMPCQRKVAFRIPSSYVSSGKKVNEFFDIGTVNMQIIFEKETRDCIHR

SEQ ID NO: 349 - protein, 138 aa, Homo sapiens
MKAVLFLFLTILSISNGMRLQAVQVKGQLKCGDKPASGVKVKLWDEDDGPDPDDVLSEGQTDRDGNFNLEGSTRELTSIDPVLKIYHDCDDGIKPGKRKVKLRIPKQYISAGSTAKKPFDIGVLNLEAIFAKEERDLI

SEQ ID NO: 350 - protein, 139 aa, Homo sapiens
MKVVIVLLALLSGVVSFRSQSVAVRGRLMCGSNPASNVRIKLWEEDSGPDPDDLLDQGYTDQNGDFLLQGDTVELTPIDPVFKVYHDCDDGLKPGKRKLKFKIPQSYITNGKTPKKVFDIGTLNLETIFHHEERELIVS

SEQ ID NO: 351 - protein, 133 aa, Homo sapiens
MHFWLLFLILGVVTFSTAMRQQAVAVKGKLLCGTAPARNIRVKLWDEDTGPDPDDLLAQGYTDDQGMFMLQGDTAELTTIDPVFKAYHDCNDNLKLGQRKLKLKIPSSYISEGKTAKKVFDIGVLNLETIFPV

SEQ ID NO: 352 - protein, 132 aa, Homo sapiens
MQLAYAGHKCVWIHGTVRCHKDPSRNLNVEVRVYDRDGLSIAKIIDPDDLMGVTFTSEDGSFQLDGCGEDIDWIPGIPNNPEPYLQILHYCNRQTGEIIKLPPFGIFVPNTYEVGIVDLDLPIQASSAKNNT

SEQ ID NO: 353 - protein, 139 aa, Homo sapiens
MDHQIVVAFLLILLSLRIYGKSECMQIVGTLVCPQKSMLAGNVQIDLKDEDSLPWETHDQMGRTWSHSDGSFMISGCGADFGPFNEPDPYIIIEHKCPSVLRSTIGNIGSTRKTQFALTKVFMPKILNIGKVFLDDSDL